SEQUENCE LISTING (142 sequences)

SEQ ID NO: 1 - protein, 19 aa, Artificial sequence
  Synthetic
MEWSWVFLFFLSVTTGVHS

SEQ ID NO: 2 - protein, 19 aa, Homo sapiens
MEFGLSWVFLVALLRGVQC

SEQ ID NO: 3 - protein, 27 aa, Mus musculus
NQTSVAPFPGNQNISASPNPSNATTRG

SEQ ID NO: 4 - protein, 30 aa, Homo sapiens
SETTTGTSSNSSQSTSNSGLAPNPTNATTK

SEQ ID NO: 5 - protein, 206 aa, Mus musculus
ALVALSQPRVQCHASRYPVAVDCSWTPLQAPNSTRSTSFIATYRLGVATQQQSQPCLQRSPQASRCTIPDVHLFSTVPYMLNVTAVHPGGASSSLLAFVAERIIKPDPPEGVRLRTAGQRLQVLWHPPASWPFPDIFSLKYRLRYRRRGASHFRQVGPIEATTFTLRNSKPHAKYCIQVSAQDLTDYGKPSDWSLPGQVESAPHKP

SEQ ID NO: 6 - protein, 203 aa, Homo sapiens
ALTLPRVQCRASRYPIAVDCSWTLPPAPNSTSPVSFIATYRLGMAARGHSWPCLQQTPTSTSCTITDVQLFSMAPYVLNVTAVHPWGSSSSFVPFITEHIIKPDPPEGVRLSPLAERQLQVQWEPPGSWPFPEIFSLKYWIRYKRQGAARFHRVGPIEATSFILRAVRPRARYYVQVAAQDLTDYGELSDWSLPATATMSLGK

SEQ ID NO: 7 - protein, 11 aa, Artificial sequence
  Synthetic
GGVPGVGVPGV

SEQ ID NO: 8 - protein, 15 aa, Artificial sequence
  Synthetic
GGGGSGGGGSGGGGS

SEQ ID NO: 9 - protein, 207 aa, Mus musculus
GFPTDPLSLQELRREFTVSLYLARKLLSEVQGYVHSFAESRLPGVNLDLLPLGYHLPNVSLTFQAWHHLSDSERLCFLATTLRPFPAMLGGLGTQGTWTSSEREQLWAMRLDLRDLHRHLRFQVLAAGFKCSKEEEDKEEEEEEEEEEKKLPLGALGGPNQVSSQVSWPQLLYTYQLLHSLELVLSRAVRDLLLLSLPRRPGSAWDS

SEQ ID NO: 10 - protein, 214 aa, Homo sapiens
GFPRPPGRPQLSLQELRREFTVSLHLARKLLSEVRGQAHRFAESHLPGVNLYLLPLGEQLPDVSLTFQAWRRLSDPERLCFISTTLQPFHALLGGLGTQGRWTNMERMQLWAMRLDLRDLQRHLRFQVLAAGFNLPEEEEEEEEEEEEERKGLLPGALGSAGPAQVSWPQLLSTYRLLHSLELVLSRAVRELLLLSKAGHSVWPLGFPTLSPQP

SEQ ID NO: 11 - protein, 222 aa, Mus musculus
GCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 12 - protein, 230 aa, Homo sapiens
KSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 222 aa, Mus musculus
GCKPCICTVPEVSSVFIFPPKPKDVLTITLTPKVTCVVVAISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 14 - protein, 230 aa, Homo sapiens
KSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 149 aa, Mus musculus
SGWLLEVPNGPWRSLTFYPAWLTVSEGANATFTCSLSNWSEDLMLNWNRLSPSNQTEKQAAFCNGLSQPVQDARFQIIQLPNRHDFHMNILDTRRNDSGIYLCGAISLHPKAKIEESPGAELVVTERILETSTRYPSPSPKPEGRFQGM

SEQ ID NO: 16 - protein, 150 aa, Homo sapiens [sequence omitted]

SEQ ID NO: 17 - protein, 170 aa, Mus musculus
SGWLLEVPNGPWRSLTFYPAWLTVSEGANATFTCSLSNWSEDLMLNWNRLSPSNQTEKQAAFCNGLSQPVQDARFQIIQLPNRHDFHMNILDTRRNDSGIYLCGAISLHPKAKIEESPGAELVVTERILETSTRYPSPSPKPEGRFQGMVIGIMSALVGIPVLLLLAWAL

SEQ ID NO: 18 - protein, 171 aa, Homo sapiens
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVI

SEQ ID NO: 19 - protein, 220 aa, Mus musculus
TITAPKDLYVVEYGSNVTMECRFPVERELDLLALVVYWEKEDEQVIQFVAGEEDLKPQHSNFRGRASLPKDQLLKGNAALQITDVKLQDAGVYCCIISYGGADYKRITLKVNAPYRKINQRISVDPATSEHELICQAEGYPEAEVIWTNSDHQPVSGKRSVTTSRTEGMLLNVTSSLRVNATANDVFYCTFWRSQPGQNHTAELIIPELPATHPPQNRTH

SEQ ID NO: 20 - protein, 219 aa, Homo sapiens
TVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNER

SEQ ID NO: 21 - protein, 241 aa, Mus musculus
TITAPKDLYVVEYGSNVTMECRFPVERELDLLALVVYWEKEDEQVIQFVAGEEDLKPQHSNFRGRASLPKDQLLKGNAALQITDVKLQDAGVYCCIISYGGADYKRITLKVNAPYRKINQRISVDPATSEHELICQAEGYPEAEVIWTNSDHQPVSGKRSVTTSRTEGMLLNVTSSLRVNATANDVFYCTFWRSQPGQNHTAELIIPELPATHPPQNRTHWVLLGSILLFLIVVSTVLLFL

SEQ ID NO: 22 - protein, 241 aa, Homo sapiens
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIF

SEQ ID NO: 23 - protein, 202 aa, Mus musculus
LFTVTAPKEVYTVDVGSSVSLECDFDRRECTELEGIRASLQKVENDTSLQSERATLLEEQLPLGKALFHIPSVQVRDSGQYRCLVICGAAWDYKYLTVKVKASYMRIDTRILEVPGTGEVQLTCQARGYPLAEVSWQNVSVPANTSHIRTPEGLYQVTSVLRLKPQPSRNFSCMFWNAHMKELTSAIIDPLSRMEPKVPRTW

SEQ ID NO: 24 - protein, 201 aa, Homo sapiens
LFTVTVPKELYIIEHGSNVTLECNFDTGSHVNLGAITASLQKVENDTSPHRERATLLEEQLPLGKASFHIPQVQVRDEGQYQCIIIYGVAWDYKYLTLKVKASYRKINTHILKVPETDEVELTCQATGYPLAEVSWPNVSVPANTSHSRTPEGLYQVTSVLRLKPPPGRNFSCVFWNTHVRELTLASIDLQSQMEPRTHPT

SEQ ID NO: 25 - protein, 223 aa, Mus musculus
LFTVTAPKEVYTVDVGSSVSLECDFDRRECTELEGIRASLQKVENDTSLQSERATLLEEQLPLGKALFHIPSVQVRDSGQYRCLVICGAAWDYKYLTVKVKASYMRIDTRILEVPGTGEVQLTCQARGYPLAEVSWQNVSVPANTSHIRTPEGLYQVTSVLRLKPQPSRNFSCMFWNAHMKELTSAIIDPLSRMEPKVPRTWPLHVFIPACTIALIFLAIVII

SEQ ID NO: 26 - protein, 222 aa, Homo sapiens
LFTVTVPKELYIIEHGSNVTLECNFDTGSHVNLGAITASLQKVENDTSPHRERATLLEEQLPLGKASFHIPQVQVRDEGQYQCIIIYGVAWDYKYLTLKVKASYRKINTHILKVPETDEVELTCQATGYPLAEVSWPNVSVPANTSHSRTPEGLYQVTSVLRLKPPPGRNFSCVFWNTHVRELTLASIDLQSQMEPRTHPTWLLHIFIPFCIIAFIFIATVI

SEQ ID NO: 27 - protein, 209 aa, Mus musculus
VDEQLSKSVKDKVLLPCRYNSPHEDESEDRIYWQKHDKVVLSVIAGKLKVWPEYKNRTLYDNTTYSLIILGLVLSDRGTYSCVVQKKERGTYEVKHLALVKLSIKADFSTPNITESGNPSADTKRITCFASGGFPKPRFSWLENGRELPGINTTISQDPESELYTISSQLDFNTTRNHTIKCLIKYGDAHVSEDFTWEKPPEDPPDSKN

SEQ ID NO: 28 - protein, 208 aa, Homo sapiens [sequence omitted]

SEQ ID NO: 29 - protein, 231 aa, Mus musculus
VDEQLSKSVKDKVLLPCRYNSPHEDESEDRIYWQKHDKVVLSVIAGKLKVWPEYKNRTLYDNTTYSLIILGLVLSDRGTYSCVVQKKERGTYEVKHLALVKLSIKADFSTPNITESGNPSADTKRITCFASGGFPKPRFSWLENGRELPGINTTISQDPESELYTISSQLDFNTTRNHTIKCLIKYGDAHVSEDFTWEKPPEDPPDSKNTLVLFGAGFGAVITVVVIVVII

SEQ ID NO: 30 - protein, 229 aa, Homo sapiens
VIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNLLPSWAITLISVNGIFVICCL

SEQ ID NO: 31 - protein, 221 aa, Mus musculus
VSVETQAYFNGTAYLPCPFTKAQNISLSELVVFWQDQQKLVLYEHYLGTEKLDSVNAKYLGRTSFDRNNWTLRLHNVQIKDMGSYDCFIQKKPPTGSIILQQTLTELSVIANFSEPEIKLAQNVTGNSGINLTCTSKQGHPKPKKMYFLITNSTNEYGDNMQISQDNVTELFSISNSLSLSFPDGVWHMTVVCVLETESMKISSKPLNFTQEFPSPQTYWK

SEQ ID NO: 32 - protein, 224 aa, Homo sapiens
APLKIQAYFNETADLPCQFANSQNQSLSELVVFWQDQENLVLNEVYLGKEKFDSVHSKYMGRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGVMQKSQDNVTELYDVSISLSVSFPDVTSNMTIFCILETDKTRLLSSPFSIELEDPQPPPDHIP

SEQ ID NO: 33 - protein, 242 aa, Mus musculus
VSVETQAYFNGTAYLPCPFTKAQNISLSELVVFWQDQQKLVLYEHYLGTEKLDSVNAKYLGRTSFDRNNWTLRLHNVQIKDMGSYDCFIQKKPPTGSIILQQTLTELSVIANFSEPEIKLAQNVTGNSGINLTCTSKQGHPKPKKMYFLITNSTNEYGDNMQISQDNVTELFSISNSLSLSFPDGVWHMTVVCVLETESMKISSKPLNFTQEFPSPQTYWKEITASVTVALLLVMLLIIVCH

SEQ ID NO: 34 - protein, 245 aa, Homo sapiens
APLKIQAYFNETADLPCQFANSQNQSLSELVVFWQDQENLVLNEVYLGKEKFDSVHSKYMGRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGVMQKSQDNVTELYDVSISLSVSFPDVTSNMTIFCILETDKTRLLSSPFSIELEDPQPPPDHIPWITAVLPTVIICVMVFCLILW

SEQ ID NO: 35 - protein, 126 aa, Mus musculus
EAIQVTQPSVVLASSHGVASFPCEYSPSHNTDEVRVTVLRQTNDQMTEVCATTFTEKNTVGFLDYPFCSGTFNESRVNLTIQGLRAVDTGLYLCKVELMYPPPYFVGMGNGTQIYVIDPEPCPDSD

SEQ ID NO: 36 - protein, 126 aa, Homo sapiens [sequence omitted]

SEQ ID NO: 37 - protein, 147 aa, Mus musculus
EAIQVTQPSVVLASSHGVASFPCEYSPSHNTDEVRVTVLRQTNDQMTEVCATTFTEKNTVGFLDYPFCSGTFNESRVNLTIQGLRAVDTGLYLCKVELMYPPPYFVGMGNGTQIYVIDPEPCPDSDFLLWILVAVSLGLFFYSFLVS

SEQ ID NO: 38 - protein, 147 aa, Homo sapiens
KAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDFLLWILAAVSSGLFFYSFLLT

SEQ ID NO: 39 - protein, 126 aa, Homo sapiens
KAMHVAQPAVVLASSRGIASFVCEYASPGKYTEVRVTVLRQADSQVTEVCAATYMMGNELTFLDDSICTGTSSGNQVNLTIQGLRAMDTGLYICKVELMYPPPYYEGIGNGTQIYVIDPEPCPDSD

SEQ ID NO: 40 - protein, 147 aa, Homo sapiens
KAMHVAQPAVVLASSRGIASFVCEYASPGKYTEVRVTVLRQADSQVTEVCAATYMMGNELTFLDDSICTGTSSGNQVNLTIQGLRAMDTGLYICKVELMYPPPYYEGIGNGTQIYVIDPEPCPDSDFLLWILAAVSSGLFFYSFLLT

SEQ ID NO: 41 - protein, 420 aa, Mus musculus
SGPGKELPVVWAQEGAPVHLPCSLKSPNLDPNFLRRGGVIWQHQPDSGQPTPIPALDLHQGMPSPRQPAPGRYTVLSVAPGGLRSGRQPLHPHVQLEERGLQRGDFSLWLRPALRTDAGEYHATVRLPNRALSCSLRLRVGQASMIASPSGVLKLSDWVLLNCSFSRPDRPVSVHWFQGQNRVPVYNSPRHFLAETFLLLPQVSPLDSGTWGCVLTYRDGFNVSITYNLKVLGLEPVAPLTVYAAEGSRVELPCHLPPGVGTPSLLIAKWTPPGGGPELPVAGKSGNFTLHLEAVGLAQAGTYTCSIHLQGQQLNATVTLAVITVTPKSFGLPGSRGKLLCEVTPASGKERFVWRPLNNLSRSCPGPVLEIQEARLLAERWQCQLYEGQRLLGATVYAAESSSGAHSARRISGDLKGGHL

SEQ ID NO: 42 - protein, 422 aa, Homo sapiens
VPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHL

SEQ ID NO: 43 - protein, 441 aa, Mus musculus
SGPGKELPVVWAQEGAPVHLPCSLKSPNLDPNFLRRGGVIWQHQPDSGQPTPIPALDLHQGMPSPRQPAPGRYTVLSVAPGGLRSGRQPLHPHVQLEERGLQRGDFSLWLRPALRTDAGEYHATVRLPNRALSCSLRLRVGQASMIASPSGVLKLSDWVLLNCSFSRPDRPVSVHWFQGQNRVPVYNSPRHFLAETFLLLPQVSPLDSGTWGCVLTYRDGFNVSITYNLKVLGLEPVAPLTVYAAEGSRVELPCHLPPGVGTPSLLIAKWTPPGGGPELPVAGKSGNFTLHLEAVGLAQAGTYTCSIHLQGQQLNATVTLAVITVTPKSFGLPGSRGKLLCEVTPASGKERFVWRPLNNLSRSCPGPVLEIQEARLLAERWQCQLYEGQRLLGATVYAAESSSGAHSARRISGDLKGGHLVLVLILGALSLFLLVAGAFGF

SEQ ID NO: 44 - protein, 443 aa, Homo sapiens
VPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHLLLFLILGVLSLLLLVTGAFGF

SEQ ID NO: 45 - protein, 174 aa, Mus musculus
RSLENAYVFEVGKNAYLPCSYTLSTPGALVPMCWGKGFCPWSQCTNELLRTDERNVTYQKSSRYQLKGDLNKGDVSLIIKNVTLDDHGTYCCRIQFPGLMNDKKLELKLDIKAAKVTPAQTAHGDSTTASPRTLTTERNGSETQTLVTLHNNNGTKISTWADEIKDSGETIRTA

SEQ ID NO: 46 - protein, 181 aa, Homo sapiens
SEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPAKVTPAPTRQRDFTAAFPRMLTTRGHGPAETQTLGSLPDINLTQISTLANELRDSRLANDLRDSGATIRIG

SEQ ID NO: 47 - protein, 195 aa, Mus musculus
RSLENAYVFEVGKNAYLPCSYTLSTPGALVPMCWGKGFCPWSQCTNELLRTDERNVTYQKSSRYQLKGDLNKGDVSLIIKNVTLDDHGTYCCRIQFPGLMNDKKLELKLDIKAAKVTPAQTAHGDSTTASPRTLTTERNGSETQTLVTLHNNNGTKISTWADEIKDSGETIRTAIHIGVGVSAGLTLALIIGVLI

SEQ ID NO: 48 - protein, 202 aa, Homo sapiens
SEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPAKVTPAPTRQRDFTAAFPRMLTTRGHGPAETQTLGSLPDINLTQISTLANELRDSRLANDLRDSGATIRIGIYIGAGICAGLALALIFGALI

SEQ ID NO: 49 - protein, 120 aa, Mus musculus
TIDTKRNISAEEGGSVILQCHFSSDTAEVTQVDWKQQDQLLAIYSVDLGWHVASVFSDRVVPGPSLGLTFQSLTMNDTGEYFCTYHTYPGGIYKGRIFLKVQESSDDRNGLAQFQTAPLG

SEQ ID NO: 50 - protein, 120 aa, Homo sapiens
MMTGTIETTGNISAEKGGSIILQCHLSSTTAQVTQVNWEQQDQLLAICNADLGWHISPSFKDRVAPGPGLGLTLQSLTVNDTGEYFCIYHTYPDGTYTGRIFLEVLESSVAEHGARFQIP

SEQ ID NO: 51 - protein, 141 aa, Mus musculus
TIDTKRNISAEEGGSVILQCHFSSDTAEVTQVDWKQQDQLLAIYSVDLGWHVASVFSDRVVPGPSLGLTFQSLTMNDTGEYFCTYHTYPGGIYKGRIFLKVQESSDDRNGLAQFQTAPLGGTMAAVLGLICLMVTGVTVLA

SEQ ID NO: 52 - protein, 141 aa, Homo sapiens
MMTGTIETTGNISAEKGGSIILQCHLSSTTAQVTQVNWEQQDQLLAICNADLGWHISPSFKDRVAPGPGLGLTLQSLTVNDTGEYFCIYHTYPDGTYTGRIFLEVLESSVAEHGARFQIPLLGAMAATLVVICTAVIVVVA

SEQ ID NO: 53 - protein, 154 aa, Mus musculus
EKATKRNDEECPVQLTITRNSKQSARTGELFKIQCPVKYCVHRPNVTWCKHNGTICVPLEVSPQLYTSWEENQSVPVFVLHFKPIHLSDNGSYSCSTNFNSQVINSHSVTIHVRERTQNSSEHPLITVSDIPDATNASGPSTMEERPGRTWLLY

SEQ ID NO: 54 - protein, 127 aa, Homo sapiens
KESCDVQLYIKRQSEHSILAGDPFELECPVKYCANRPHVTWCKLNGTTCVKLEDRQTSWKEEKNISFFILHFEPVLPNDNGSYRCSANFQSNLIESHSTTLYVTDVKSASERPSKDEMASRPWLLYR

SEQ ID NO: 55 - protein, 175 aa, Mus musculus
EKATKRNDEECPVQLTITRNSKQSARTGELFKIQCPVKYCVHRPNVTWCKHNGTICVPLEVSPQLYTSWEENQSVPVFVLHFKPIHLSDNGSYSCSTNFNSQVINSHSVTIHVRERTQNSSEHPLITVSDIPDATNASGPSTMEERPGRTWLLYTLLPLGALLLLLACVCLLCFL

SEQ ID NO: 56 - protein, 148 aa, Homo sapiens
KESCDVQLYIKRQSEHSILAGDPFELECPVKYCANRPHVTWCKLNGTTCVKLEDRQTSWKEEKNISFFILHFEPVLPNDNGSYRCSANFQSNLIESHSTTLYVTDVKSASERPSKDEMASRPWLLYRLLPLGGLPLLITTCFCLFCCL

SEQ ID NO: 57 - protein, 154 aa, Mus musculus
AFKVTTPYSLYVCPEGQNATLTCRILGPVSKGHDVTIYKTWYLSSRGEVQMCKEHRPIRNFTLQHLQHHGSHLKANASHDQPQKHGLELASDHHGNFSITLRNVTPRDSGLYCCLVIELKNHHPEQRFYGSMELQVQAGKGSGSTCMASNEQDS

SEQ ID NO: 58 - protein, 159 aa, Homo sapiens
AFKVATPYSLYVCPEGQNVTLTCRLLGPVDKGHDVTFYKTWYRSSRGEVQTCSERRPIRNLTFQDLHLHHGGHQAANTSHDLAQRHGLESASDHHGNFSITMRNLTLLDSGLYCCLVVEIRHHHSEHRVHGAMELQVQTGKDAPSNCVVYPSSSQDSEN

SEQ ID NO: 59 - protein, 182 aa, Mus musculus
AFKVTTPYSLYVCPEGQNATLTCRILGPVSKGHDVTIYKTWYLSSRGEVQMCKEHRPIRNFTLQHLQHHGSHLKANASHDQPQKHGLELASDHHGNFSITLRNVTPRDSGLYCCLVIELKNHHPEQRFYGSMELQVQAGKGSGSTCMASNEQDSDSITAAALATGACIVGILCLPLILLLVY

SEQ ID NO: 60 - protein, 185 aa, Homo sapiens
AFKVATPYSLYVCPEGQNVTLTCRLLGPVDKGHDVTFYKTWYRSSRGEVQTCSERRPIRNLTFQDLHLHHGGHQAANTSHDLAQRHGLESASDHHGNFSITMRNLTLLDSGLYCCLVVEIRHHHSEHRVHGAMELQVQTGKDAPSNCVVYPSSSQDSENITAAALATGACIVGILCLPLILLLVY

SEQ ID NO: 61 - protein, 50 aa, Mus musculus
NQTSVAPFPGNQNISASPNPSNATTRGGGSSLQSTAGLLALSLSLLHLYC

SEQ ID NO: 62 - protein, 54 aa, Homo sapiens
SETTTGTSSNSSQSTSNSGLAPNPTNATTKAAGGALQSTASLFVVSLSLLHLYS

SEQ ID NO: 63 - protein, 22 aa, Mus musculus
TLVLFGAGFGAVITVVVIVVII

SEQ ID NO: 64 - protein, 21 aa, Homo sapiens
LLPSWAITLISVNGIFVICCL

SEQ ID NO: 65 - protein, 60 aa, Mus musculus
TLVLFGAGFGAVITVVVIVVIIKCFCKHRSCFRRNEASRETNNSLTFGPEEALAEQTVFL

SEQ ID NO: 66 - protein, 46 aa, Homo sapiens
LLPSWAITLISVNGIFVICCLTYCFAPRCRERRRNERLRRESVRPV

SEQ ID NO: 67 - protein, 443 aa, Artificial sequence
  Synthetic
MEWSWVFLFFLSVTTGVHSALVALSQPRVQCHASRYPVAVDCSWTPLQAPNSTRSTSFIATYRLGVATQQQSQPCLQRSPQASRCTIPDVHLFSTVPYMLNVTAVHPGGASSSLLAFVAERIIKPDPPEGVRLRTAGQRLQVLWHPPASWPFPDIFSLKYRLRYRRRGASHFRQVGPIEATTFTLRNSKPHAKYCIQVSAQDLTDYGKPSDWSLPGQVESAPHKPGGVPGVGVPGVGFPTDPLSLQELRREFTVSLYLARKLLSEVQGYVHSFAESRLPGVNLDLLPLGYHLPNVSLTFQAWHHLSDSERLCFLATTLRPFPAMLGGLGTQGTWTSSEREQLWAMRLDLRDLHRHLRFQVLAAGFKCSKEEEDKEEEEEEEEEEKKLPLGALGGPNQVSSQVSWPQLLYTYQLLHSLELVLSRAVRDLLLLSLPRRPGSAWDS

SEQ ID NO: 68 - protein, 447 aa, Artificial sequence
  Synthetic
MEWSWVFLFFLSVTTGVHSALTLPRVQCRASRYPIAVDCSWTLPPAPNSTSPVSFIATYRLGMAARGHSWPCLQQTPTSTSCTITDVQLFSMAPYVLNVTAVHPWGSSSSFVPFITEHIIKPDPPEGVRLSPLAERQLQVQWEPPGSWPFPEIFSLKYWIRYKRQGAARFHRVGPIEATSFILRAVRPRARYYVQVAAQDLTDYGELSDWSLPATATMSLGKGGVPGVGVPGVGFPRPPGRPQLSLQELRREFTVSLHLARKLLSEVRGQAHRFAESHLPGVNLYLLPLGEQLPDVSLTFQAWRRLSDPERLCFISTTLQPFHALLGGLGTQGRWTNMERMQLWAMRLDLRDLQRHLRFQVLAAGFNLPEEEEEEEEEEEEERKGLLPGALGSAGPAQVSWPQLLSTYRLLHSLELVLSRAVRELLLLSKAGHSVWPLGFPTLSPQP

SEQ ID NO: 69 - protein, 673 aa, Artificial sequence
  Synthetic
MEWSWVFLFFLSVTTGVHSALVALSQPRVQCHASRYPVAVDCSWTPLQAPNSTRSTSFIATYRLGVATQQQSQPCLQRSPQASRCTIPDVHLFSTVPYMLNVTAVHPGGASSSLLAFVAERIIKPDPPEGVRLRTAGQRLQVLWHPPASWPFPDIFSLKYRLRYRRRGASHFRQVGPIEATTFTLRNSKPHAKYCIQVSAQDLTDYGKPSDWSLPGQVESAPHKPGGVPGVGVPGVGFPTDPLSLQELRREFTVSLYLARKLLSEVQGYVHSFAESRLPGVNLDLLPLGYHLPNVSLTFQAWHHLSDSERLCFLATTLRPFPAMLGGLGTQGTWTSSEREQLWAMRLDLRDLHRHLRFQVLAAGFKCSKEEEDKEEEEEEEEEEKKLPLGALGGPNQVSSQVSWPQLLYTYQLLHSLELVLSRAVRDLLLLSLPRRPGSAWDSGGGGAGGGGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 70 - protein, 677 aa, Artificial sequence
  Synthetic
MEWSWVFLFFLSVTTGVHSALTLPRVQCRASRYPIAVDCSWTLPPAPNSTSPVSFIATYRLGMAARGHSWPCLQQTPTSTSCTITDVQLFSMAPYVLNVTAVHPWGSSSSFVPFITEHIIKPDPPEGVRLSPLAERQLQVQWEPPGSWPFPEIFSLKYWIRYKRQGAARFHRVGPIEATSFILRAVRPRARYYVQVAAQDLTDYGELSDWSLPATATMSLGKGGVPGVGVPGVGFPRPPGRPQLSLQELRREFTVSLHLARKLLSEVRGQAHRFAESHLPGVNLYLLPLGEQLPDVSLTFQAWRRLSDPERLCFISTTLQPFHALLGGLGTQGRWTNMERMQLWAMRLDLRDLQRHLRFQVLAAGFNLPEEEEEEEEEEEEERKGLLPGALGSAGPAQVSWPQLLSTYRLLHSLELVLSRAVRELLLLSKAGHSVWPLGFPTLSPQPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 71 - protein, 673 aa, Artificial sequence
  Synthetic
MEWSWVFLFFLSVTTGVHSALVALSQPRVQCHASRYPVAVDCSWTPLQAPNSTRSTSFIATYRLGVATQQQSQPCLQRSPQASRCTIPDVHLFSTVPYMLNVTAVHPGGASSSLLAFVAERIIKPDPPEGVRLRTAGQRLQVLWHPPASWPFPDIFSLKYRLRYRRRGASHFRQVGPIEATTFTLRNSKPHAKYCIQVSAQDLTDYGKPSDWSLPGQVESAPHKPGGVPGVGVPGVGFPTDPLSLQELRREFTVSLYLARKLLSEVQGYVHSFAESRLPGVNLDLLPLGYHLPNVSLTFQAWHHLSDSERLCFLATTLRPFPAMLGGLGTQGTWTSSEREQLWAMRLDLRDLHRHLRFQVLAAGFKCSKEEEDKEEEEEEEEEEKKLPLGALGGPNQVSSQVSWPQLLYTYQLLHSLELVLSRAVRDLLLLSLPRRPGSAWDSGGGGAGGGGCKPCICTVPEVSSVFIFPPKPKDVLTITLTPKVTCVVVAISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 72 - protein, 677 aa, Artificial sequence
  Synthetic
MEWSWVFLFFLSVTTGVHSALTLPRVQCRASRYPIAVDCSWTLPPAPNSTSPVSFIATYRLGMAARGHSWPCLQQTPTSTSCTITDVQLFSMAPYVLNVTAVHPWGSSSSFVPFITEHIIKPDPPEGVRLSPLAERQLQVQWEPPGSWPFPEIFSLKYWIRYKRQGAARFHRVGPIEATSFILRAVRPRARYYVQVAAQDLTDYGELSDWSLPATATMSLGKGGVPGVGVPGVGFPRPPGRPQLSLQELRREFTVSLHLARKLLSEVRGQAHRFAESHLPGVNLYLLPLGEQLPDVSLTFQAWRRLSDPERLCFISTTLQPFHALLGGLGTQGRWTNMERMQLWAMRLDLRDLQRHLRFQVLAAGFNLPEEEEEEEEEEEEERKGLLPGALGSAGPAQVSWPQLLSTYRLLHSLELVLSRAVRELLLLSKAGHSVWPLGFPTLSPQPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 73 - protein, 677 aa, Artificial sequence
  Synthetic
MEWSWVFLFFLSVTTGVHSALVALSQPRVQCHASRYPVAVDCSWTPLQAPNSTRSTSFIATYRLGVATQQQSQPCLQRSPQASRCTIPDVHLFSTVPYMLNVTAVHPGGASSSLLAFVAERIIKPDPPEGVRLRTAGQRLQVLWHPPASWPFPDIFSLKYRLRYRRRGASHFRQVGPIEATTFTLRNSKPHAKYCIQVSAQDLTDYGKPSDWSLPGQVESAPHKPGGGGSGGGGSGGGGSGFPTDPLSLQELRREFTVSLYLARKLLSEVQGYVHSFAESRLPGVNLDLLPLGYHLPNVSLTFQAWHHLSDSERLCFLATTLRPFPAMLGGLGTQGTWTSSEREQLWAMRLDLRDLHRHLRFQVLAAGFKCSKEEEDKEEEEEEEEEEKKLPLGALGGPNQVSSQVSWPQLLYTYQLLHSLELVLSRAVRDLLLLSLPRRPGSAWDSGGGGAGGGGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 74 - protein, 681 aa, Artificial sequence
  Synthetic
MEWSWVFLFFLSVTTGVHSALTLPRVQCRASRYPIAVDCSWTLPPAPNSTSPVSFIATYRLGMAARGHSWPCLQQTPTSTSCTITDVQLFSMAPYVLNVTAVHPWGSSSSFVPFITEHIIKPDPPEGVRLSPLAERQLQVQWEPPGSWPFPEIFSLKYWIRYKRQGAARFHRVGPIEATSFILRAVRPRARYYVQVAAQDLTDYGELSDWSLPATATMSLGKGGGGSGGGGSGGGGSGFPRPPGRPQLSLQELRREFTVSLHLARKLLSEVRGQAHRFAESHLPGVNLYLLPLGEQLPDVSLTFQAWRRLSDPERLCFISTTLQPFHALLGGLGTQGRWTNMERMQLWAMRLDLRDLQRHLRFQVLAAGFNLPEEEEEEEEEEEEERKGLLPGALGSAGPAQVSWPQLLSTYRLLHSLELVLSRAVRELLLLSKAGHSVWPLGFPTLSPQPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 75 - protein, 677 aa, Artificial sequence
  Synthetic
MEWSWVFLFFLSVTTGVHSALVALSQPRVQCHASRYPVAVDCSWTPLQAPNSTRSTSFIATYRLGVATQQQSQPCLQRSPQASRCTIPDVHLFSTVPYMLNVTAVHPGGASSSLLAFVAERIIKPDPPEGVRLRTAGQRLQVLWHPPASWPFPDIFSLKYRLRYRRRGASHFRQVGPIEATTFTLRNSKPHAKYCIQVSAQDLTDYGKPSDWSLPGQVESAPHKPGGGGSGGGGSGGGGSGFPTDPLSLQELRREFTVSLYLARKLLSEVQGYVHSFAESRLPGVNLDLLPLGYHLPNVSLTFQAWHHLSDSERLCFLATTLRPFPAMLGGLGTQGTWTSSEREQLWAMRLDLRDLHRHLRFQVLAAGFKCSKEEEDKEEEEEEEEEEKKLPLGALGGPNQVSSQVSWPQLLYTYQLLHSLELVLSRAVRDLLLLSLPRRPGSAWDSGGGGAGGGGCKPCICTVPEVSSVFIFPPKPKDVLTITLTPKVTCVVVAISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 76 - protein, 681 aa, Artificial sequence
  Synthetic
MEWSWVFLFFLSVTTGVHSALTLPRVQCRASRYPIAVDCSWTLPPAPNSTSPVSFIATYRLGMAARGHSWPCLQQTPTSTSCTITDVQLFSMAPYVLNVTAVHPWGSSSSFVPFITEHIIKPDPPEGVRLSPLAERQLQVQWEPPGSWPFPEIFSLKYWIRYKRQGAARFHRVGPIEATSFILRAVRPRARYYVQVAAQDLTDYGELSDWSLPATATMSLGKGGGGSGGGGSGGGGSGFPRPPGRPQLSLQELRREFTVSLHLARKLLSEVRGQAHRFAESHLPGVNLYLLPLGEQLPDVSLTFQAWRRLSDPERLCFISTTLQPFHALLGGLGTQGRWTNMERMQLWAMRLDLRDLQRHLRFQVLAAGFNLPEEEEEEEEEEEEERKGLLPGALGSAGPAQVSWPQLLSTYRLLHSLELVLSRAVRELLLLSKAGHSVWPLGFPTLSPQPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 77 - protein, 703 aa, Artificial sequence
  Synthetic
MEWSWVFLFFLSVTTGVHSSETTTGTSSNSSQSTSNSGLAPNPTNATTKALVALSQPRVQCHASRYPVAVDCSWTPLQAPNSTRSTSFIATYRLGVATQQQSQPCLQRSPQASRCTIPDVHLFSTVPYMLNVTAVHPGGASSSLLAFVAERIIKPDPPEGVRLRTAGQRLQVLWHPPASWPFPDIFSLKYRLRYRRRGASHFRQVGPIEATTFTLRNSKPHAKYCIQVSAQDLTDYGKPSDWSLPGQVESAPHKPGGVPGVGVPGVGFPTDPLSLQELRREFTVSLYLARKLLSEVQGYVHSFAESRLPGVNLDLLPLGYHLPNVSLTFQAWHHLSDSERLCFLATTLRPFPAMLGGLGTQGTWTSSEREQLWAMRLDLRDLHRHLRFQVLAAGFKCSKEEEDKEEEEEEEEEEKKLPLGALGGPNQVSSQVSWPQLLYTYQLLHSLELVLSRAVRDLLLLSLPRRPGSAWDSGGGGAGGGGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 78 - protein, 707 aa, Artificial sequence
  Synthetic
MEWSWVFLFFLSVTTGVHSSETTTGTSSNSSQSTSNSGLAPNPTNATTKALTLPRVQCRASRYPIAVDCSWTLPPAPNSTSPVSFIATYRLGMAARGHSWPCLQQTPTSTSCTITDVQLFSMAPYVLNVTAVHPWGSSSSFVPFITEHIIKPDPPEGVRLSPLAERQLQVQWEPPGSWPFPEIFSLKYWIRYKRQGAARFHRVGPIEATSFILRAVRPRARYYVQVAAQDLTDYGELSDWSLPATATMSLGKGGVPGVGVPGVGFPRPPGRPQLSLQELRREFTVSLHLARKLLSEVRGQAHRFAESHLPGVNLYLLPLGEQLPDVSLTFQAWRRLSDPERLCFISTTLQPFHALLGGLGTQGRWTNMERMQLWAMRLDLRDLQRHLRFQVLAAGFNLPEEEEEEEEEEEEERKGLLPGALGSAGPAQVSWPQLLSTYRLLHSLELVLSRAVRELLLLSKAGHSVWPLGFPTLSPQPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 79 - protein, 703 aa, Artificial sequence
  Synthetic
MEWSWVFLFFLSVTTGVHSSETTTGTSSNSSQSTSNSGLAPNPTNATTKALVALSQPRVQCHASRYPVAVDCSWTPLQAPNSTRSTSFIATYRLGVATQQQSQPCLQRSPQASRCTIPDVHLFSTVPYMLNVTAVHPGGASSSLLAFVAERIIKPDPPEGVRLRTAGQRLQVLWHPPASWPFPDIFSLKYRLRYRRRGASHFRQVGPIEATTFTLRNSKPHAKYCIQVSAQDLTDYGKPSDWSLPGQVESAPHKPGGVPGVGVPGVGFPTDPLSLQELRREFTVSLYLARKLLSEVQGYVHSFAESRLPGVNLDLLPLGYHLPNVSLTFQAWHHLSDSERLCFLATTLRPFPAMLGGLGTQGTWTSSEREQLWAMRLDLRDLHRHLRFQVLAAGFKCSKEEEDKEEEEEEEEEEKKLPLGALGGPNQVSSQVSWPQLLYTYQLLHSLELVLSRAVRDLLLLSLPRRPGSAWDSGGGGAGGGGCKPCICTVPEVSSVFIFPPKPKDVLTITLTPKVTCVVVAISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 80 - protein, 711 aa, Artificial sequence
  Synthetic
MEWSWVFLFFLSVTTGVHSSETTTGTSSNSSQSTSNSGLAPNPTNATTKALTLPRVQCRASRYPIAVDCSWTLPPAPNSTSPVSFIATYRLGMAARGHSWPCLQQTPTSTSCTITDVQLFSMAPYVLNVTAVHPWGSSSSFVPFITEHIIKPDPPEGVRLSPLAERQLQVQWEPPGSWPFPEIFSLKYWIRYKRQGAARFHRVGPIEATSFILRAVRPRARYYVQVAAQDLTDYGELSDWSLPATATMSLGKGGGGSGGGGSGGGGSGFPRPPGRPQLSLQELRREFTVSLHLARKLLSEVRGQAHRFAESHLPGVNLYLLPLGEQLPDVSLTFQAWRRLSDPERLCFISTTLQPFHALLGGLGTQGRWTNMERMQLWAMRLDLRDLQRHLRFQVLAAGFNLPEEEEEEEEEEEEERKGLLPGALGSAGPAQVSWPQLLSTYRLLHSLELVLSRAVRELLLLSKAGHSVWPLGFPTLSPQPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 81 - protein, 707 aa, Artificial sequence
  Synthetic
MEWSWVFLFFLSVTTGVHSSETTTGTSSNSSQSTSNSGLAPNPTNATTKALVALSQPRVQCHASRYPVAVDCSWTPLQAPNSTRSTSFIATYRLGVATQQQSQPCLQRSPQASRCTIPDVHLFSTVPYMLNVTAVHPGGASSSLLAFVAERIIKPDPPEGVRLRTAGQRLQVLWHPPASWPFPDIFSLKYRLRYRRRGASHFRQVGPIEATTFTLRNSKPHAKYCIQVSAQDLTDYGKPSDWSLPGQVESAPHKPGGGGSGGGGSGGGGSGFPTDPLSLQELRREFTVSLYLARKLLSEVQGYVHSFAESRLPGVNLDLLPLGYHLPNVSLTFQAWHHLSDSERLCFLATTLRPFPAMLGGLGTQGTWTSSEREQLWAMRLDLRDLHRHLRFQVLAAGFKCSKEEEDKEEEEEEEEEEKKLPLGALGGPNQVSSQVSWPQLLYTYQLLHSLELVLSRAVRDLLLLSLPRRPGSAWDSGGGGAGGGGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 82 - protein, 711 aa, Artificial sequence
  Synthetic
MEWSWVFLFFLSVTTGVHSSETTTGTSSNSSQSTSNSGLAPNPTNATTKALTLPRVQCRASRYPIAVDCSWTLPPAPNSTSPVSFIATYRLGMAARGHSWPCLQQTPTSTSCTITDVQLFSMAPYVLNVTAVHPWGSSSSFVPFITEHIIKPDPPEGVRLSPLAERQLQVQWEPPGSWPFPEIFSLKYWIRYKRQGAARFHRVGPIEATSFILRAVRPRARYYVQVAAQDLTDYGELSDWSLPATATMSLGKGGGGSGGGGSGGGGSGFPRPPGRPQLSLQELRREFTVSLHLARKLLSEVRGQAHRFAESHLPGVNLYLLPLGEQLPDVSLTFQAWRRLSDPERLCFISTTLQPFHALLGGLGTQGRWTNMERMQLWAMRLDLRDLQRHLRFQVLAAGFNLPEEEEEEEEEEEEERKGLLPGALGSAGPAQVSWPQLLSTYRLLHSLELVLSRAVRELLLLSKAGHSVWPLGFPTLSPQPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 83 - protein, 707 aa, Artificial sequence
  Synthetic
MEWSWVFLFFLSVTTGVHSSETTTGTSSNSSQSTSNSGLAPNPTNATTKALVALSQPRVQCHASRYPVAVDCSWTPLQAPNSTRSTSFIATYRLGVATQQQSQPCLQRSPQASRCTIPDVHLFSTVPYMLNVTAVHPGGASSSLLAFVAERIIKPDPPEGVRLRTAGQRLQVLWHPPASWPFPDIFSLKYRLRYRRRGASHFRQVGPIEATTFTLRNSKPHAKYCIQVSAQDLTDYGKPSDWSLPGQVESAPHKPGGGGSGGGGSGGGGSGFPTDPLSLQELRREFTVSLYLARKLLSEVQGYVHSFAESRLPGVNLDLLPLGYHLPNVSLTFQAWHHLSDSERLCFLATTLRPFPAMLGGLGTQGTWTSSEREQLWAMRLDLRDLHRHLRFQVLAAGFKCSKEEEDKEEEEEEEEEEKKLPLGALGGPNQVSSQVSWPQLLYTYQLLHSLELVLSRAVRDLLLLSLPRRPGSAWDSGGGGAGGGGCKPCICTVPEVSSVFIFPPKPKDVLTITLTPKVTCVVVAISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 84 - protein, 711 aa, Artificial sequence; identical to SEQ ID NO: 80
  Synthetic

SEQ ID NO: 85 - protein, 449 aa, Artificial sequence
  Synthetic
MEFGLSWVFLVALLRGVQCALTLPRVQCRASRYPIAVDCSWTLPPAPNSTSPVSFIATYRLGMAARGHSWPCLQQTPTSTSCTITDVQLFSMAPYVLNVTAVHPWGSSSSFVPFITEHIIKPDPPEGVRLSPLAERQLQVQWEPPGSWPFPEIFSLKYWIRYKRQGAARFHRVGPIEATSFILRAVRPRARYYVQVAAQDLTDYGELSDWSLPATATMSLGKGGVPGVGVPGVGFPRPPGRPQLSLQELRREFTVSLHLARKLLSEVRGQAHRFAESHLPGVNLYLLPLGEQLPDVSLTFQAWRRLSDPERLCFISTTLQPFHALLGGLGTQGRWTNMERMQLWAMRLDLRDLQRHLRFQVLAAGFNLPEEEEEEEEEEEEERKGLLPGALGSALQGPAQVSWPQLLSTYRLLHSLELVLSRAVRELLLLSKAGHSVWPLGFPTLSPQP

SEQ ID NO: 86 - protein, 677 aa, Artificial sequence
  Synthetic
MEFGLSWVFLVALLRGVQCALTLPRVQCRASRYPIAVDCSWTLPPAPNSTSPVSFIATYRLGMAARGHSWPCLQQTPTSTSCTITDVQLFSMAPYVLNVTAVHPWGSSSSFVPFITEHIIKPDPPEGVRLSPLAERQLQVQWEPPGSWPFPEIFSLKYWIRYKRQGAARFHRVGPIEATSFILRAVRPRARYYVQVAAQDLTDYGELSDWSLPATATMSLGKGGVPGVGVPGVGFPRPPGRPQLSLQELRREFTVSLHLARKLLSEVRGQAHRFAESHLPGVNLYLLPLGEQLPDVSLTFQAWRRLSDPERLCFISTTLQPFHALLGGLGTQGRWTNMERMQLWAMRLDLRDLQRHLRFQVLAAGFNLPEEEEEEEEEEEEERKGLLPGALGSAGPAQVSWPQLLSTYRLLHSLELVLSRAVRELLLLSKAGHSVWPLGFPTLSPQPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 87 - protein, 677 aa, Artificial sequence
  Synthetic
MEFGLSWVFLVALLRGVQCALTLPRVQCRASRYPIAVDCSWTLPPAPNSTSPVSFIATYRLGMAARGHSWPCLQQTPTSTSCTITDVQLFSMAPYVLNVTAVHPWGSSSSFVPFITEHIIKPDPPEGVRLSPLAERQLQVQWEPPGSWPFPEIFSLKYWIRYKRQGAARFHRVGPIEATSFILRAVRPRARYYVQVAAQDLTDYGELSDWSLPATATMSLGKGGVPGVGVPGVGFPRPPGRPQLSLQELRREFTVSLHLARKLLSEVRGQAHRFAESHLPGVNLYLLPLGEQLPDVSLTFQAWRRLSDPERLCFISTTLQPFHALLGGLGTQGRWTNMERMQLWAMRLDLRDLQRHLRFQVLAAGFNLPEEEEEEEEEEEEERKGLLPGALGSAGPAQVSWPQLLSTYRLLHSLELVLSRAVRELLLLSKAGHSVWPLGFPTLSPQPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 88 - protein, 700 aa, Artificial sequence
  Synthetic
MEWSWVFLFFLSVTTGVHSNQTSVAPFPGNQNISASPNPSNATTRGALVALSQPRVQCHASRYPVAVDCSWTPLQAPNSTRSTSFIATYRLGVATQQQSQPCLQRSPQASRCTIPDVHLFSTVPYMLNVTAVHPGGASSSLLAFVAERIIKPDPPEGVRLRTAGQRLQVLWHPPASWPFPDIFSLKYRLRYRRRGASHFRQVGPIEATTFTLRNSKPHAKYCIQVSAQDLTDYGKPSDWSLPGQVESAPHKPGGVPGVGVPGVGFPTDPLSLQELRREFTVSLYLARKLLSEVQGYVHSFAESRLPGVNLDLLPLGYHLPNVSLTFQAWHHLSDSERLCFLATTLRPFPAMLGGLGTQGTWTSSEREQLWAMRLDLRDLHRHLRFQVLAAGFKCSKEEEDKEEEEEEEEEEKKLPLGALGGPNQVSSQVSWPQLLYTYQLLHSLELVLSRAVRDLLLLSLPRRPGSAWDSGGGGAGGGGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 89 - protein, 707 aa, Artificial sequence
  Synthetic
MEFGLSWVFLVALLRGVQCSETTTGTSSNSSQSTSNSGLAPNPTNATTKALTLPRVQCRASRYPIAVDCSWTLPPAPNSTSPVSFIATYRLGMAARGHSWPCLQQTPTSTSCTITDVQLFSMAPYVLNVTAVHPWGSSSSFVPFITEHIIKPDPPEGVRLSPLAERQLQVQWEPPGSWPFPEIFSLKYWIRYKRQGAARFHRVGPIEATSFILRAVRPRARYYVQVAAQDLTDYGELSDWSLPATATMSLGKGGVPGVGVPGVGFPRPPGRPQLSLQELRREFTVSLHLARKLLSEVRGQAHRFAESHLPGVNLYLLPLGEQLPDVSLTFQAWRRLSDPERLCFISTTLQPFHALLGGLGTQGRWTNMERMQLWAMRLDLRDLQRHLRFQVLAAGFNLPEEEEEEEEEEEEERKGLLPGALGSAGPAQVSWPQLLSTYRLLHSLELVLSRAVRELLLLSKAGHSVWPLGFPTLSPQPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 90 - protein, 700 aa, Artificial sequence
  Synthetic
MEWSWVFLFFLSVTTGVHSNQTSVAPFPGNQNISASPNPSNATTRGALVALSQPRVQCHASRYPVAVDCSWTPLQAPNSTRSTSFIATYRLGVATQQQSQPCLQRSPQASRCTIPDVHLFSTVPYMLNVTAVHPGGASSSLLAFVAERIIKPDPPEGVRLRTAGQRLQVLWHPPASWPFPDIFSLKYRLRYRRRGASHFRQVGPIEATTFTLRNSKPHAKYCIQVSAQDLTDYGKPSDWSLPGQVESAPHKPGGVPGVGVPGVGFPTDPLSLQELRREFTVSLYLARKLLSEVQGYVHSFAESRLPGVNLDLLPLGYHLPNVSLTFQAWHHLSDSERLCFLATTLRPFPAMLGGLGTQGTWTSSEREQLWAMRLDLRDLHRHLRFQVLAAGFKCSKEEEDKEEEEEEEEEEKKLPLGALGGPNQVSSQVSWPQLLYTYQLLHSLELVLSRAVRDLLLLSLPRRPGSAWDSGGGGAGGGGCKPCICTVPEVSSVFIFPPKPKDVLTITLTPKVTCVVVAISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 91 - protein, 707 aa, Artificial sequence
  Synthetic
MEFGLSWVFLVALLRGVQCSETTTGTSSNSSQSTSNSGLAPNPTNATTKALTLPRVQCRASRYPIAVDCSWTLPPAPNSTSPVSFIATYRLGMAARGHSWPCLQQTPTSTSCTITDVQLFSMAPYVLNVTAVHPWGSSSSFVPFITEHIIKPDPPEGVRLSPLAERQLQVQWEPPGSWPFPEIFSLKYWIRYKRQGAARFHRVGPIEATSFILRAVRPRARYYVQVAAQDLTDYGELSDWSLPATATMSLGKGGVPGVGVPGVGFPRPPGRPQLSLQELRREFTVSLHLARKLLSEVRGQAHRFAESHLPGVNLYLLPLGEQLPDVSLTFQAWRRLSDPERLCFISTTLQPFHALLGGLGTQGRWTNMERMQLWAMRLDLRDLQRHLRFQVLAAGFNLPEEEEEEEEEEEEERKGLLPGALGSAGPAQVSWPQLLSTYRLLHSLELVLSRAVRELLLLSKAGHSVWPLGFPTLSPQPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 92 - protein, 700 aa, Artificial sequence; identical to SEQ ID NO: 88
  Synthetic

SEQ ID NO: 93 - protein, 707 aa, Artificial sequence; identical to SEQ ID NO: 89
  Synthetic

SEQ ID NO: 94 - protein, 700 aa, Artificial sequence; identical to SEQ ID NO: 90
  Synthetic

SEQ ID NO: 95 - protein, 707 aa, Artificial sequence; identical to SEQ ID NO: 91
  Synthetic

SEQ ID NO: 96 - protein, 501 aa, Artificial sequence
  Synthetic
MEWSWVFLFFLSVTTGVHSALVALSQPRVQCHASRYPVAVDCSWTPLQAPNSTRSTSFIATYRLGVATQQQSQPCLQRSPQASRCTIPDVHLFSTVPYMLNVTAVHPGGASSSLLAFVAERIIKPDPPEGVRLRTAGQRLQVLWHPPASWPFPDIFSLKYRLRYRRRGASHFRQVGPIEATTFTLRNSKPHAKYCIQVSAQDLTDYGKPSDWSLPGQVESAPHKPGGVPGVGVPGVGFPTDPLSLQELRREFTVSLYLARKLLSEVQGYVHSFAESRLPGVNLDLLPLGYHLPNVSLTFQAWHHLSDSERLCFLATTLRPFPAMLGGLGTQGTWTSSEREQLWAMRLDLRDLHRHLRFQVLAAGFKCSKEEEDKEEEEEEEEEEKKLPLGALGGPNQVSSQVSWPQLLYTYQLLHSLELVLSRAVRDLLLLSLPRRPGSAWDSGGGGAGGGNQTSVAPFPGNQNISASPNPSNATTRGGGSSLQSTAGLLALSLSLLHLYC

SEQ ID NO: 97 - protein, 509 aa, Artificial sequence
  Synthetic
MEFGLSWVFLVALLRGVQCALTLPRVQCRASRYPIAVDCSWTLPPAPNSTSPVSFIATYRLGMAARGHSWPCLQQTPTSTSCTITDVQLFSMAPYVLNVTAVHPWGSSSSFVPFITEHIIKPDPPEGVRLSPLAERQLQVQWEPPGSWPFPEIFSLKYWIRYKRQGAARFHRVGPIEATSFILRAVRPRARYYVQVAAQDLTDYGELSDWSLPATATMSLGKGGVPGVGVPGVGFPRPPGRPQLSLQELRREFTVSLHLARKLLSEVRGQAHRFAESHLPGVNLYLLPLGEQLPDVSLTFQAWRRLSDPERLCFISTTLQPFHALLGGLGTQGRWTNMERMQLWAMRLDLRDLQRHLRFQVLAAGFNLPEEEEEEEEEEEEERKGLLPGALGSAGPAQVSWPQLLSTYRLLHSLELVLSRAVRELLLLSKAGHSVWPLGFPTLSPQPGGGGAGGGSETTTGTSSNSSQSTSNSGLAPNPTNATTKAAGGALQSTASLFVVSLSLLHLYS

SEQ ID NO: 98 - protein, 500 aa, Artificial sequence
  Synthetic
MEWSWVFLFFLSVTTGVHSNQTSVAPFPGNQNISASPNPSNATTRGALVALSQPRVQCHASRYPVAVDCSWTPLQAPNSTRSTSFIATYRLGVATQQQSQPCLQRSPQASRCTIPDVHLFSTVPYMLNVTAVHPGGASSSLLAFVAERIIKPDPPEGVRLRTAGQRLQVLWHPPASWPFPDIFSLKYRLRYRRRGASHFRQVGPIEATTFTLRNSKPHAKYCIQVSAQDLTDYGKPSDWSLPGQVESAPHKPGGVPGVGVPGVGFPTDPLSLQELRREFTVSLYLARKLLSEVQGYVHSFAESRLPGVNLDLLPLGYHLPNVSLTFQAWHHLSDSERLCFLATTLRPFPAMLGGLGTQGTWTSSEREQLWAMRLDLRDLHRHLRFQVLAAGFKCSKEEEDKEEEEEEEEEEKKLPLGALGGPNQVSSQVSWPQLLYTYQLLHSLELVLSRAVRDLLLLSLPRRPGSAWDSGGGGAGGGTLVLFGAGFGAVITVVVIVVII

SEQ ID NO: 99 - protein, 506 aa, Artificial sequence
  Synthetic
MEFGLSWVFLVALLRGVQCSETTTGTSSNSSQSTSNSGLAPNPTNATTKALTLPRVQCRASRYPIAVDCSWTLPPAPNSTSPVSFIATYRLGMAARGHSWPCLQQTPTSTSCTITDVQLFSMAPYVLNVTAVHPWGSSSSFVPFITEHIIKPDPPEGVRLSPLAERQLQVQWEPPGSWPFPEIFSLKYWIRYKRQGAARFHRVGPIEATSFILRAVRPRARYYVQVAAQDLTDYGELSDWSLPATATMSLGKGGVPGVGVPGVGFPRPPGRPQLSLQELRREFTVSLHLARKLLSEVRGQAHRFAESHLPGVNLYLLPLGEQLPDVSLTFQAWRRLSDPERLCFISTTLQPFHALLGGLGTQGRWTNMERMQLWAMRLDLRDLQRHLRFQVLAAGFNLPEEEEEEEEEEEEERKGLLPGALGSAGPAQVSWPQLLSTYRLLHSLELVLSRAVRELLLLSKAGHSVWPLGFPTLSPQPGGGGAGGGLLPSWAITLISVNGIFVICCL

SEQ ID NO: 100 - protein, 538 aa, Artificial sequence
  Synthetic
MEWSWVFLFFLSVTTGVHSNQTSVAPFPGNQNISASPNPSNATTRGALVALSQPRVQCHASRYPVAVDCSWTPLQAPNSTRSTSFIATYRLGVATQQQSQPCLQRSPQASRCTIPDVHLFSTVPYMLNVTAVHPGGASSSLLAFVAERIIKPDPPEGVRLRTAGQRLQVLWHPPASWPFPDIFSLKYRLRYRRRGASHFRQVGPIEATTFTLRNSKPHAKYCIQVSAQDLTDYGKPSDWSLPGQVESAPHKPGGVPGVGVPGVGFPTDPLSLQELRREFTVSLYLARKLLSEVQGYVHSFAESRLPGVNLDLLPLGYHLPNVSLTFQAWHHLSDSERLCFLATTLRPFPAMLGGLGTQGTWTSSEREQLWAMRLDLRDLHRHLRFQVLAAGFKCSKEEEDKEEEEEEEEEEKKLPLGALGGPNQVSSQVSWPQLLYTYQLLHSLELVLSRAVRDLLLLSLPRRPGSAWDSGGGGAGGGTLVLFGAGFGAVITVVVIVVIIKCFCKHRSCFRRNEASRETNNSLTFGPEEALAEQTVFL

SEQ ID NO: 101 - protein, 523 aa, Artificial sequence
  Synthetic
MEFGLSWVFLVALLRGVQCSETTTGTSSNSSQSTSNSGLAPNPTNATTKALTLPRVQCRASRYPIAVDCSWTLPPAPNSTSPVSFIATYRLGMAARGHSWPCLQQTPTSTSCTITDVQLFSMAPYVLNVTAVHPWGSSSSFVPFITEHIIKPDPPEGVRLSPLAERQLQVQWEPPGSWPFPEIFSLKYWIRYKRQGAARFHRVGPIEATSFILRAVRPRARYYVQVAAQDLTDYGELSDWSLPATATMSLGKGGVPGVGVPGVGFPRPPGRPQLSLQELRREFTVSLHLARKLLSEVRGQAHRFAESHLPGVNLYLLPLGEQLPDVSLTFQAWRRLSDPERLCFISTTLQPFHALLGGLGTQGRWTNMERMQLWAMRLDLRDLQRHLRFQVLAAGFNLPEEEEEEEEEEEEERKGLLPGALGSAGPAQVSWPQLLSTYRLLHSLELVLSRAVRELLLLSKAGHSVWPLGFPTLSPQPLLPSWAITLISVNGIFVICCLTYCFAPRCRERRRNERLRRESVRPV

SEQ ID NO: 102 - protein, 31 aa, Artificial sequence
  Synthetic
SETTTGTSSNSSQSTSNSGLAPNPTNATTKV

SEQ ID NO: 103 - protein, 29 aa, Artificial sequence
  Synthetic
SETTTGTSSNSSQSTSNSGLAPNPTNATT

SEQ ID NO: 104 - protein, 28 aa, Artificial sequence
  Synthetic
SETTTGTSSNSSQSTSNSGLAPNPTNAT

SEQ ID NO: 105 - protein, 27 aa, Artificial sequence
  Synthetic
NQTSVAPFPGNQNISASPNPTNATTRG

SEQ ID NO: 106 - protein, 26 aa, Artificial sequence
  Synthetic
NQTSVAPFPGNQNISASPNPTNATTR

SEQ ID NO: 107 - protein, 25 aa, Artificial sequence
  Synthetic
NQTSVAPFPGNQNISASPNPTNATT

SEQ ID NO: 108 - protein, 24 aa, Artificial sequence
  Synthetic
NQTSVAPFPGNQNISASPNPTNAT

SEQ ID NO: 109 - protein, 18 aa, Artificial sequence
  Synthetic
GGSSRSSSSGGGGSGGGG

SEQ ID NO: 110 - protein, 15 aa, Artificial sequence
  Synthetic
GSGRSGGGGSGGGGS

SEQ ID NO: 111 - protein, 8 aa, Artificial sequence
  Synthetic
GGGGAGGG

SEQ ID NO: 112 - protein, 14 aa, Artificial sequence
  Synthetic
AAAGGMPPAAAGGM

SEQ ID NO: 113 - protein, 6 aa, Artificial sequence
  Synthetic
AAAGGM

SEQ ID NO: 114 - protein, 9 aa, Artificial sequence
  Synthetic
PPAAAGGMM

SEQ ID NO: 115 - protein, 25 aa, Artificial sequence
  Synthetic
FILPILGAVLALLLLLTLLALLLLV

SEQ ID NO: 116 - protein, 27 aa, Artificial sequence
  Synthetic
IYLIIGICGGGSLLMVEFVALLVFYIT

SEQ ID NO: 117 - protein, 22 aa, Artificial sequence
  Synthetic
ALVVIPIIFGILFAILLVLVFT

SEQ ID NO: 118 - protein, 20 aa, Artificial sequence
  Synthetic
ISGATAGVPTLLLGLVLPAP

SEQ ID NO: 119 - protein, 24 aa, Artificial sequence
  Synthetic
LLLGVSVSCIVILAVCLLCYVSIT

SEQ ID NO: 120 - protein, 24 aa, Artificial sequence
  Synthetic
VAGVVIIVILLILTGAGLAAYFFY

SEQ ID NO: 121 - protein, 25 aa, Artificial sequence
  Synthetic
FLFTPVVVACMSIMALLLLLLLLLL

SEQ ID NO: 122 - protein, 30 aa, Artificial sequence
  Synthetic
VVVISAILALVVLTIISLIILIMLWQKKPR

SEQ ID NO: 123 - protein, 26 aa, Artificial sequence
  Synthetic
ELTVAAAVLVLLVIVSISLIVLVVTW

SEQ ID NO: 124 - protein, 24 aa, Artificial sequence
  Synthetic
LTYFGGAVASTIGLIMGGTLLALL

SEQ ID NO: 125 - protein, 23 aa, Artificial sequence
  Synthetic
TVLLPLVIFFGLCLLSLLFIGLM

SEQ ID NO: 126 - protein, 23 aa, Artificial sequence
  Synthetic
LLVLYFASSLIIPAIGMIIYFAR

SEQ ID NO: 127 - protein, 19 aa, Artificial sequence
  Synthetic
MELGLSWIFLLAILKGVQC

SEQ ID NO: 128 - protein, 19 aa, Artificial sequence
  Synthetic
MELGLRWVFLVAILEGVQC

SEQ ID NO: 129 - protein, 19 aa, Artificial sequence
  Synthetic
MKHLWFFLLLVAAPRWVLS

SEQ ID NO: 130 - protein, 19 aa, Artificial sequence
  Synthetic
MDWTWRILFLVAAATGAHS

SEQ ID NO: 131 - protein, 19 aa, Artificial sequence
  Synthetic
MDWTWRFLFVVAAATGVQS

SEQ ID NO: 132 - protein, 19 aa, Artificial sequence
  Synthetic
MEFGLSWLFLVAILKGVQC

SEQ ID NO: 133 - protein, 26 aa, Artificial sequence
  Synthetic
MDLLHKNMKHLWFFLLLVAAPRWVLS

SEQ ID NO: 134 - protein, 22 aa, Artificial sequence
  Synthetic
VKQTLNFDLLKLAGDVESNPGP

SEQ ID NO: 135 - DNA, 2022 bp, Artificial sequence
  Synthetic
atggaatggagctgggtctttctcttcttcctgtcagtaacgactggtgtccactccgctctggtggctctgtcccagcctagagtgcagtgccacgcctccagataccctgtggccgtggactgctcttggacccctctgcaggcccctaactctacccggtccacctcctttatcgccacctacagactgggcgtggccacccagcagcagtctcagccttgtctgcagagatcccctcaggcctcccggtgcaccatccctgatgtgcacctgttctccaccgtgccctacatgctgaacgtgaccgctgtgcatcctggcggcgcttccagttctctgctggcctttgtggccgagcggatcatcaagcctgatccccctgaaggcgtgcggctgagaaccgctggacagagactgcaggtgctgtggcaccctcctgcctcttggcccttcccagacatcttcagcctgaagtaccggctgcggtacaggcggagaggcgccagccactttagacaagtgggccccatcgaggccaccaccttcaccctgcggaactccaagccccacgccaagtactgcatccaggtgtccgcccaggacctgaccgactacggcaagccttctgactggtccctgcccggccaggtggaatctgctcctcataagcctggcggagtgcctggcgtgggcgtgccaggcgtgggatttcctacagatcccctgagcctgcaggaactgcggcgcgagtttaccgtgtccctgtacctggcccggaagctgctgtctgaggtgcagggctacgtgcactccttcgccgagagtagactgcccggcgtgaacctggatctgctgcccctgggctaccatctgcccaatgtgtccctgacatttcaggcctggcaccacctgtccgactccgagagactgtgtttcctggctaccaccctgaggcctttccccgctatgctgggaggactgggaacccagggcacctggacatcctctgagcgggaacagctgtgggccatgagactggacctgcgggacctgcaccggcacctgagattccaggtgctggccgctggcttcaagtgctccaaagaggaagaggacaaagaagaggaagaagaagaagaggaagaggaaaagaaactgcctctgggcgccctgggaggccccaatcaggtgtccagtcaggtgtcatggcctcagctgctgtacacctaccagctgctgcactccctggaactggtgctgtctagagccgtgcgcgacctgctgctgctgtccctgccaagaaggcctggctctgcctgggactctggcggaggtggtgcaggaggcggtggatgtaagccttgcatatgtacagtcccagaagtatcatctgtcttcatcttccccccaaagcccaaggatgtgctcaccattactctgactcctaaggtcacgtgtgttgtggtagccatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaatag

SEQ ID NO: 136 - DNA, 2031 bp, Artificial sequence
  Synthetic
atggaatggagctgggtctttctcttcttcctgtcagtaacgactggtgtccactccgctctggtggctctgtcccagcctagagtgcagtgccacgcctccagataccctgtggccgtggactgctcttggacccctctgcaggcccctaactctacccggtccacctcctttatcgccacctacagactgggcgtggccacccagcagcagtctcagccttgtctgcagagatcccctcaggcctcccggtgcaccatccctgatgtgcacctgttctccaccgtgccctacatgctgaacgtgaccgctgtgcatcctggcggcgcttccagttctctgctggcctttgtggccgagcggatcatcaagcctgatccccctgaaggcgtgcggctgagaaccgctggacagagactgcaggtgctgtggcaccctcctgcctcttggcccttcccagacatcttcagcctgaagtaccggctgcggtacaggcggagaggcgccagccactttagacaagtgggccccatcgaggccaccaccttcaccctgcggaactccaagccccacgccaagtactgcatccaggtgtccgcccaggacctgaccgactacggcaagccttctgactggtccctgcccggccaggtggaatctgctcctcataagcctggcggaggcggatctggtggcggaggtagtggcggcggaggctcttttcctacagatcccctgagcctgcaggaactgcggcgcgagtttaccgtgtccctgtacctggcccggaagctgctgtctgaggtgcagggctacgtgcactccttcgccgagagtagactgcccggcgtgaacctggatctgctgcccctgggctaccatctgcccaatgtgtccctgacatttcaggcctggcaccacctgtccgactccgagagactgtgtttcctggctaccaccctgaggcctttccccgctatgctgggaggactgggaacccagggcacctggacatcctctgagcgggaacagctgtgggccatgagactggacctgcgggacctgcaccggcacctgagattccaggtgctggccgctggcttcaagtgctccaaagaggaagaggacaaagaagaggaagaagaagaagaggaagaggaaaagaaactgcctctgggcgccctgggaggccccaatcaggtgtccagtcaggtgtcatggcctcagctgctgtacacctaccagctgctgcactccctggaactggtgctgtctagagccgtgcgcgacctgctgctgctgtccctgccaagaaggcctggctctgcctgggactctggcggaggtggtgcaggaggcggtggatgtaagccttgcatatgtacagtcccagaagtatcatctgtcttcatcttccccccaaagcccaaggatgtgctcaccattactctgactcctaaggtcacgtgtgttgtggtagccatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaatag

SEQ ID NO: 137 - DNA, 2112 bp, Artificial sequence
  Synthetic
atggaatggagctgggtctttctcttcttcctgtcagtaacgactggtgtccactcctctgaaacgacaacaggaacctccagcaattcttcccaatcaacatccaactccggactagcacccaatccaacaaacgccaccaccaaggctctggtggctctgtcccagcctagagtgcagtgccacgcctccagataccctgtggccgtggactgctcttggacccctctgcaggcccctaactctacccggtccacctcctttatcgccacctacagactgggcgtggccacccagcagcagtctcagccttgtctgcagagatcccctcaggcctcccggtgcaccatccctgatgtgcacctgttctccaccgtgccctacatgctgaacgtgaccgctgtgcatcctggcggcgcttccagttctctgctggcctttgtggccgagcggatcatcaagcctgatccccctgaaggcgtgcggctgagaaccgctggacagagactgcaggtgctgtggcaccctcctgcctcttggcccttcccagacatcttcagcctgaagtaccggctgcggtacaggcggagaggcgccagccactttagacaagtgggccccatcgaggccaccaccttcaccctgcggaactccaagccccacgccaagtactgcatccaggtgtccgcccaggacctgaccgactacggcaagccttctgactggtccctgcccggccaggtggaatctgctcctcataagcctggcggagtgcctggcgtgggcgtgccaggcgtgggatttcctacagatcccctgagcctgcaggaactgcggcgcgagtttaccgtgtccctgtacctggcccggaagctgctgtctgaggtgcagggctacgtgcactccttcgccgagagtagactgcccggcgtgaacctggatctgctgcccctgggctaccatctgcccaatgtgtccctgacatttcaggcctggcaccacctgtccgactccgagagactgtgtttcctggctaccaccctgaggcctttccccgctatgctgggaggactgggaacccagggcacctggacatcctctgagcgggaacagctgtgggccatgagactggacctgcgggacctgcaccggcacctgagattccaggtgctggccgctggcttcaagtgctccaaagaggaagaggacaaagaagaggaagaagaagaagaggaagaggaaaagaaactgcctctgggcgccctgggaggccccaatcaggtgtccagtcaggtgtcatggcctcagctgctgtacacctaccagctgctgcactccctggaactggtgctgtctagagccgtgcgcgacctgctgctgctgtccctgccaagaaggcctggctctgcctgggactctggcggaggtggtgcaggaggcggtggatgtaagccttgcatatgtacagtcccagaagtatcatctgtcttcatcttccccccaaagcccaaggatgtgctcaccattactctgactcctaaggtcacgtgtgttgtggtagccatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaatag

SEQ ID NO: 138 - DNA, 2124 bp, Artificial sequence
  Synthetic
atggaatggagctgggtctttctcttcttcctgtcagtaacgactggtgtccactcctccgagacaaccaccggcacctccagcaactcctcccagtccacctccaactctggcctggcccccaaccctaccaacgccaccacaaaggctctgaccctgcctcgggtgcagtgccgggcttccagataccctatcgccgtggactgctcctggaccctgccccctgctcctaactccacctcccccgtgtcctttatcgccacctacagactgggcatggccgccagaggccactcttggccttgtctgcagcagacccctacctccaccagctgcaccatcaccgacgtgcagctgttctccatggccccctacgtgctgaacgtgaccgctgtgcatccttggggctcctccagctccttcgtgcccttcatcaccgagcacatcatcaagcccgaccctccagaaggcgtgcggctgtctcctctggctgaaagacagctgcaggtgcagtgggagcctcctggctcttggcccttcccagagatcttcagcctgaagtactggatccggtacaagcggcagggcgctgccagattccatagagtgggccctatcgaggccacctccttcatcctgagagccgtgcggcccagagcccggtactatgtgcaggtggcagcccaggacctgaccgactatggcgagctgtctgactggtccctgcctgccaccgccaccatgagtctgggaaaaggcggagtgcctggcgtgggcgtgccaggcgtgggatttcctagacctcctggccggcctcagctgtccctgcaggaactgcggagagaattcaccgtgtctctgcacctggcccggaagctgctgtctgaagtgcggggacaggcccacagattcgccgagtctcatctgcccggcgtgaacctgtacctgctgcctctgggagagcagctgcctgacgtgtccctgacattccaggcttggcggagactgtccgaccctgagcggctgtgcttcatctccaccaccctgcagcctttccacgctctgctgggcggactgggcacacagggcagatggaccaacatggaacggatgcagctgtgggccatgcggctggacctgagggatctgcagcggcacctgaggttccaggtgctggccgctggcttcaacctgcccgaggaagaagaggaagaggaagaagaagaagaggaagagaggaagggcctgctgcctggcgctctgggatctgctggaccagctcaggtgtcctggccacagctgctgagcacctaccggctgctgcactccctggaactggtgctgtctagggccgtgcgggaactgctgctgctgtccaaggctggccactctgtgtggccactgggcttccctaccctgtcccctcagcctaagtcctgcgacaaaactcacacatgcccaccgtgcccagcacctgaactcctggggggaccgtcagtcttcctcttccccccaaaacccaaggacaccctcatgatctcccggacccctgaggtcacatgcgtggtggtggcagtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 139 - DNA, 32 bp, Artificial sequence
  Synthetic
aatctactcgagatcaccggtaggagggccaa

SEQ ID NO: 140 - DNA, 32 bp, Artificial sequence
  Synthetic
atgtatgatatcatgtcgagctagcttaggaa

SEQ ID NO: 141 - DNA, 5373 bp, Artificial sequence
  Synthetic
tagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggtaccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattggatccactcgagtggagctcgcgactagtcgattcgaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgactagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagctttttgcaaaagcctaggcctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccattcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatctggctagcgatgaccctgctgattggttcgctgaccatttccgggtgcgggacggcgttaccagaaactcagaaggttcgtccaaccaaaccgactctgacggcagtttacgagagagatgatagggtctgcttcagtaagccagatgctacacaattaggcttgtacatattgtcgttagaacgcggctacaattaatacataaccttatgtatcatacacatacgatttaggtgacactatagaatacacggaattaattc

SEQ ID NO: 142 - protein, 216 aa, Homo sapiens
GFPRPPGRPQLSLQELRREFTVSLHLARKLLSEVRGQAHRFAESHLPGVNLYLLPLGEQLPDVSLTFQAWRRLSDPERLCFISTTLQPFHALLGGLGTQGRWTNMERMQLWAMRLDLRDLQRHLRFQVLAAGFNLPEEEEEEEEEEEEERKGLLPGALGSALQGPAQVSWPQLLSTYRLLHSLELVLSRAVRELLLLSKAGHSVWPLGFPTLSPQP

(19) United States
(12) Patent Application Publication
(10) Pub. No.: US 2019/0119353 A1
(43) Pub. Date: Apr. 25, 2019

(54) IMMUNOTHERAPEUTICS FOR CANCER AND AUTOIMMUNE DISEASES

(71) Applicants: CHILDREN'S RESEARCH INSTITUTE, CHILDREN'S NATIONAL MEDICAL CENTER, Washington, DC (US); THE OHIO STATE UNIVERSITY, Columbus, OH (US)

(72) Inventors: Xue-Feng BAI, Columbus, OH (US); Yang LIU, Washington, DC (US); Pan ZHENG, Washington, DC (US); Jin-Qing LIU, Columbus, OH (US)

(21) Appl. No.: 15/525,013

(22) PCT Filed: Nov. 5, 2015
(86) PCT No.: PCT/US15/59217

§ 371 Date: May 5, 2017

Related U.S. Application Data
(60) Provisional application No. 62/076,182, filed on Nov. 6, 2014.

(51) Int. Cl. C07K 14/705; C07K 14/54; A61K 48/00; A61K 35/12; A61K 38/00

(52) U.S. Cl. CPC C07K 14/70596; C07K 14/54; A61K 48/005; C12N 2750/14143; A61K 38/00; C07K 2319/00; A61K 35/12

(57) ABSTRACT

Chimeric fusion proteins and polynucleotides encoding the chimeric fusion proteins are provided for the treatment of proliferative disorders, automimmune diseases and alloimmune responses. The chimeric fusion proteins comprise a CD24 extracellular domain, an EBV-induced 3 (EBI3) polypeptide subunit, and a p28 IL-27 polypeptide subunit, wherein the EBB polypeptide and the p28 IL-27 polypeptide subunit are covalently joined by a flexible peptide linker.

This application claims priority to U.S. Provisional Patent Application No. 62/076,182, filed Nov. 6, 2014. The entirety of all of the aforementioned applications is incorporated herein by reference.

### FIELD

This application relates generally to compositions and methods employing or expressing chimeric IL-27 fusion proteins for treatment of proliferative disorders, autoimmune diseases and alloimmune responses.

### BACKGROUND

After decades of research, cancer immunotherapy has finally emerged as a major weapon in our war against cancer. The current immunotherapeutic approach can be divided into those that directly targeting cancer cells and those that directly target the host immune system. The former includes antibodies specific for molecules that are abundantly expressed on tumor cells, such as HER-2 in breast cancer and CD19 or CD20 in leukemia. The latter includes antibodies that interact with negative checkpoint regulators, such as CTLA-4 and PD-1. The power of combination is illustrated by significant increase of response rate by combining anti-CTLA-4 and anti-PD-1 antibodies. In recent years, the number of negative checkpoint regulators has expanded to provide new targets, including PD-L1, PD-L2, B7-1, B7-2, LAG-3, TIM-3, TIGIT, BTLA, and VISTA (reviewed in Le Mercier et al., Front. Immunol., (6), Article 418, August 2015). Additional combinations are eagerly sought after by pharmaceutical companies.

IL-27 is a member of the IL-12 cytokine family that consists of an IL-12 p40-related protein subunit, which is called EBV-induced gene 3 (EBI3), and a p35-related subunit, p28. Produced by activated antigen presenting cells (APCs) such as dendritic cells (DCs) and macrophages, IL-27 signals through a heterodimeric receptor (IL-27R) consisting of the WSX-1 and the gp130 subunits in a variety of cell types including T, NK, B cells and myeloid cells. IL-27R signaling enhances the recruitment of several Jak family kinases and activation of STAT family transcription factors 1 and 3. IL-27 has been shown to have potent activity in regulating Th1, Th2, Th17 and FoxP3⁺ Treg (regulatory T cell) responses. In view of the properties of IL-27, there is a need for IL-27-based therapeutic agents for treatment of proliferative disorders, autoimmune diseases and alloimmune responses.

IL-27 is a member of the IL-12 cytokine family that consists of an IL-12 p40-related protein subunit, which is called EBV-induced gene 3 (EBI3), and a p35-related subunit, p28. Produced by activated antigen presenting cells (APCs) such as dendritic cells (DCs) and macrophages, IL-27 signals through a heterodimeric receptor (IL-27R) consisting of the WSX-1 and the gp130 subunits in a variety of cell types including T, NK, B cells and myeloid cells. IL-27R signaling enhances the recruitment of several Jak family kinases and activation of STAT family transcription factors 1 and 3. IL-27 has been shown to have potent activity in regulating Th1, Th2, Th17 and FoxP3⁺ Treg (regulatory T cell) responses. In view of the properties of IL-27, there is a need for IL-27-based therapeutic agents for treatment of proliferative disorders, autoimmune diseases and alloimmune responses.

### SUMMARY

One aspect of the present application relates to an IL-27 fusion protein comprising a CD24 extracellular domain, an EBV-induced 3 (EBI3) polypeptide, a p28 IL-27 polypeptide subunit, wherein the EBI3 polypeptide and the p28 IL-27 polypeptide subunit are covalently joined by a flexible peptide linker.

In one embodiment, the IL-27 fusion protein comprises, from amino to carboxy terminus, the CD24 extracellular domain, the EBV-induced 3 (EBI3) polypeptide subunit, the peptide linker and the p28 IL-27 polypeptide subunit.

In another embodiment, the IL-27 fusion protein comprises, from amino to carboxy terminus, the CD24 extracellular domain, the p28 IL-27 polypeptide subunit, the peptide linker and the EBV-induced 3 (EBI3) polypeptide.

In certain embodiments, the IL-27 fusion protein comprises between 2-10 or between 2-5 tandemly arranged copies of the CD24 extracellular domain.

In other embodiments, the IL-27 fusion protein may comprise between 2-5 tandemly arranged copies of the above described extracellular domains.

In another embodiment, the IL-27 fusion protein comprises an immunoglobulin Fc domain. In one embodiment, the immunoglobulin Fc domain comprises an IgG1 heavy chain constant region. In another embodiment, the immunoglobulin Fc domain comprises a mutation abrogating or eliminating binding to an Fcγ receptor. In certain particular embodiments, the fusion protein comprises an amino acid sequence according to any one of SEQ ID NOs: 77-84 or 88-101.

Another aspect of the present application relates to a pharmaceutical composition comprising an IL-27 fusion protein in accordance with the present disclosure in combination with one or more members selected from the group consisting of an anti-PD-1 agent, anti-PD-L1 agent, anti-PD-L2 agent, or combination thereof. In certain embodiments, the anti-PD-1 agent, anti-PD-L1 agent or anti-PD-L2 agent is an antibody. In other embodiments, the anti-PD-1 agent, anti-PD-L1 agent or anti-PD-L2 agent is a dominant negative mutant protein. In other embodiments, the anti-PD-1 agent, anti-PD-L1 agent or anti-PD-L2 agent is an siRNA.

Another aspect of the present application relates to a polynucleotide encoding an IL-27 fusion protein as described herein, as well as an expression vector comprising the IL-27 fusion protein encoding polynucleotide operably linked to nucleotide sequences suitable for expressing the fusion protein in a cell. Each of the polynucleotides or expression vectors additionally includes an N-terminal signal peptide sequence.

In certain embodiments, the polynucleotide or expression vector additionally includes a GPI anchor signal sequence at the carboxy-terminal end of the fusion protein sequence for anchoring the fusion protein to a cell membrane.

In other embodiments, the polynucleotide or expression vector additionally includes a transmembrane domain for anchoring the fusion protein to a cell membrane.

In certain embodiments, polynucleotide or expression vector, or an additional polynucleotide or expression vector further includes one or more sequences suitable for expressing an anti-PD-1 agent, an anti-PD-L1 agent, an anti-PD-L2 agent, or a combination thereof.

In certain embodiments, the anti-PD-1/anti-PD-L1/anti-PD-L2 sequences encode an antibody.

In other embodiments, the anti-PD-1/anti-PD-L1/anti-PD-L2 sequences encode a dominant negative mutant protein or a soluble protein comprising an extracellular domain without the transmembrane domain and/or cytoplasmic tail.

The polynucleotide or expression vector encoding the antibody, dominant negative mutant protein or soluble protein may be engineered for secretion of the anti-PD-1/anti-PD-L1/anti-PD-L2 sequences (as e.g., an antibody or Fc fusion protein), or they may be engineered with a C-terminal GPI-linked anchor signal sequence or transmembrane domain for anchoring the PD-1, PD-L1, and/or PD-L2 variant to a cell membrane.

In other embodiments, the anti-PD-1/anti-PD-L1/anti-PD-L2 sequences comprise one or more shRNAs for producing one or more siRNA.

In other embodiments, polynucleotide or expression vector, or an additional polynucleotide or expression vector further includes one or more sequences suitable for expressing an antibody directed against B7-1, B7-2, CTLA-4, LAG-3, TIM-3, TIGIT, BTLA, VISTA or a protein variant comprising an extracellular domain from B7-1, B7-2, CTLA-4, LAG-3, TIME-3, TIGIT, BTLA, VISTA or a combination thereof. The protein variant may be engineered for secretion (as e.g., an Fc fusion protein) or it may be engineered with a GPI-linked anchor signal sequence or transmembrane domain for anchoring the protein variant to a cell membrane.

The expression vector may be a viral vector or a non-viral vector.

In certain preferred embodiments, the expression vector is a recombinant adenovirus-associated virus (AAV) vector.

Another aspect of the present application relates to a cell comprising an IL-27 fusion protein encoding polynucleotide or an IL-27 fusion protein encoded expression vector, wherein the cell expresses the IL-27 fusion protein. In certain embodiments, the cell additionally includes a polynucleotide or expression vector expressing an anti-PD-1 agent, anti-PD-L1 agent, anti-PD-L2 agent, or combination thereof, wherein the anti-PD-1/PD-L1/PD-L2 agent is an antibody, a dominant negative protein or an siRNA. In preferred embodiments, the cell is a mammalian cell, such as a human or mouse cell.

Another aspect of the present application relates to a method for treating a proliferative disorder. The method comprises the step of administering to a subject in need thereof an effective amount of a IL-27 fusion protein of the present disclosure.

Another aspect of the present application relates to a method for treating an autoimmune disease or an alloimmune response. The method comprises the step of administering to a subject in need thereof an effective amount of a IL-27 fusion protein of the present disclosure.

Another aspect of the present application relates to a method for treating a proliferative disorder. The method comprises the step of administering to a subject in need thereof an effective amount of a IL-27-encoding expression vector of the present disclosure.

Another aspect of the present application relates to a method for treating an autoimmune disease or an alloimmune response. The method comprises the step of administering to a subject in need thereof an effective amount of a IL-27-encoding expression vector of the present disclosure.

In certain embodiments, the treatment methods further comprise the step of administering to the subject an effective amount of: an anti-PD-1 mAb, anti-PD-L1 mAb, anti-PD-L2 mAb, or a combination thereof; an expression vector expressing an anti-PD-1 mAb, an anti-PD-L1 mAb, an anti-PD-L2 mAb, or a combination thereof; a dominant-negative protein of PD-1, PD-L1, PD-L2, or a combination thereof; an expression vector expressing PD-1, PD-L1, PD-L2, or a combination thereof; one or more siRNAs directed against PD-1, PD-L1, or PD-L2; or an expression vector expressing one or more siRNAs directed against PD-1, PD-L1, or PD-L2.

In other embodiments, the treatment methods further comprise the step of administering to the subject an effective amount of: a mAb directed against B7-1, B7-2, CTLA-4, LAG-3, TIM-3, TIGIT, BTLA, VISTA, or a combination thereof; an expression vector expressing an mAb directed against B7-1, B7-2, CTLA-4, LAG-3, TIM-3, TIGIT, BTLA, VISTA, or a combination thereof; a dominant-negative protein or extracellular domain from of B7-1, B7-2, CTLA-4, LAG-3, TIM-3, TIGIT, BTLA, VISTA, or a combination thereof; an expression vector expressing a dominant-negative protein or extracellular domain from of B7-1, B7-2, CTLA-4, LAG-3, TIM-3, TIGIT, BTLA, VISTA, or a combination thereof; one or more siRNAs directed against B7-1, B7-2, CTLA-4, LAG-3, TIM-3, TIGIT, BTLA, VISTA, or a combination thereof; or an expression vector expressing one or more siRNAs directed against B7-1, B7-2, CTLA-4, LAG-3, TIM-3, TIGIT, BTLA, VISTA, or a combination thereof.

Another aspect of the present application relates to a method for treating a proliferative disorder. The method comprises the step of administering to a subject in need thereof an effective amount of a cell expressing an IL-27 fusion protein of the present disclosure alone, or in combination with one or more anti-PD-1 agents, anti-PD-L1 agents, anti-PD-L2 agents, anti-B7-1 agents, anti-B7-2 agents, anti-CTLA-4 agents, anti-LAG-3 agents, anti-TIM-3 agents, anti-TIGIT agents, anti-BTLA agents, anti-VISTA agents, or combination thereof, in an amount effective to treat the proliferative disorder.

Another aspect of the present application relates to a method for treating an autoimmune disease or an alloimmune response. The method comprises administering to a subject in need thereof an effective amount of a cell expressing an IL-27 fusion protein of the present disclosure alone, or in combination with one or more anti-PD-1 agents, anti-PD-L1 agents, anti-PD-L2 agents, anti-B7-1 agents, anti-B7-2 agents, anti-CTLA-4 agents, anti-LAG-3 agents, anti-TIM-3 agents, anti-TIGIT agents, anti-BTLA agents, anti-VISTA agents, or combination thereof, in an amount effective to treat the autoimmune disease or alloimmune response.

### BRIEF DESCRIPTION OF THE DRAWINGS

FIG. 1. AAV-IL-27 treatment inhibits the growth and lung metastasis of B16 melanoma. Panel A: C57BL/6 mice were injected with AAV-IL-27 or AAV-Ctrl viral vectors i.m. Mice were bled over time and the concentrations of IL-27 in sera were detected by ELISA. Panel B. 2×10⁵ B16.F10 cells were injected into C57BL/6 mice s.c. Four days later mice were treated with AAV-IL-27 or AAV-Ctrl viral vectors. The sizes of tumors were measured over time. Bars indicate SD of five tumors in each group. Data shown represent three experiments with similar results. Panel C: 2×10⁵ B16.F10 cells were injected into C57BL/6 mice i.v. Four days later mice were treated with AAV-IL-27 or AAV-Ctrl viral vectors i.m. Twenty one days after tumor cell injection, mice were sacrificed and tumor metastasis in the lungs was shown. Average weight of the lungs from each group of mice was shown in the right panel. Bars indicate SD of lung weight of four mice in each group. Data shown represent two experiments with similar results.

FIG. 2. AAV-IL-27 treatment depletes Treg cells and enhances tumor infiltration of IFN-γ producing T cells. 2×10⁵ B16.F10 cells were injected into C57BL/6 mice s.c. Four days later mice were treated with AAV-IL-27 or AAV-Ctrl viral vectors. Mice were sacrificed on day 18, and flow cytometry were used to analyze FoxP3⁺ Treg cells (Panel A) and IFN-γ-producing tumor infiltrating T cells (Panel B). Five mice per group were used for the experiments. Data shown represent at least three experiments with similar results. *P<0.05; **P<0.01; ***P<0.001 by student's t-test.

FIG. 3. AAV-IL-27 treatment induces PD-L1 expression on T cells. Panel A: Flow cytometry analysis of CD4+ and CD8+ T cells in the blood (n=3-4/group), spleens (n=7-8/group) and tumors (n=7-8/group) from mice that were treated with AAV-IL-27 (red) or AAV-Ctrl viral (blue) vectors. Average mean fluorescence intensity (MFI) of PD-L1 expression on T cells from each group of mice was shown in the right panel. Bars indicate SD of PD-L1 MFI in each group. Data shown represent two experiments with similar results. Panel B: Flow cytometry analysis of PD-1+ CD8 T cells in the tumors from mice that were treated with AAV-IL-27 or AAV-Ctrl viral vectors. Bars indicate SD of PD-1 MFI from five mice per group. Data shown represent two experiments with similar results. *P<0.05; ***P<0.001 by student's t-test.

FIG. 4. AAV-IL-27 and anti-PD1 combination therapy eliminates tumors in mice. Panel A: 2×10⁵ B16.F10 cells were injected into each C57BL6 mice s.c. Four days later mice were treated with AAV-IL-27 or AAV-Ctrl vectors i.m. Starting on day 4, mice were also treated with 300 μg/mouse of anti-PD-1 (RMP1-14) or an isotype-matched control antibody (2A3) at four-day intervals for up to four times. The sizes of tumors in each group of mice (n=5) were measured over time. Panel B: 2×10⁵ B16.F10 cells were injected into each C57BL6 mice s.c. Four days later mice were treated with AAV-IL-27 vectors i.m. Starting on day 4, mice were also treated with 300 μg/mouse of anti-PD-1(RMP1-14), or anti-CTLA4 (4F10) or a control IgG (2A3). The sizes of tumors in each group of mice (n=5) were measured over time. *P<0.05; **P<0.01 by the student's t-test. Data shown represent three experiments with similar results.

FIG. 5. AAV-IL-27 and anti-PD1 combination therapy enhances anti-tumor Th1/Tc1 responses. 2×10⁵ B16.F10 cells were injected into each C57BL6 mice s.c. Four days later mice were treated with AAV-IL-27 or AAV-Ctrl vectors i.m. Starting on day 4, mice were also treated with 300 μg/mouse of anti-PD-1 (RMP1-14) or an isotype-matched control antibody (2A3) at four-day intervals for up to four times. Eighteen days after tumor cell injection, mice were sacrificed and their tumors were isolated and analyzed for the production of IFN-γ by CD4 (Panel A) and CD8 (Panel B) T cells. Average IFN-γ-positive T cells from each group of mice (n=5) was shown in the right panel. *p<0.05; **p<0.01 by the student's t-test.

FIG. 6. AAV-IL-27 treatment eliminates FoxP3⁺ Treg cells. Mice B16 melanoma establishment and treatment protocol were the same as described in FIGS. 3 and 4. Eighteen days after tumor cell injection, mice were sacrificed and their spleens (Panel A) and tumors (Panel B) were isolated and analyzed for the expression of Foxp3 and IL-10 in CD4 T cells. Average FoxP3-positive and IL-10-positive T cells from each group of mice (n=5) was shown in the right panel. *p<0.05; **p<0.01 by the student's t-test.

FIG. 7. AAV-IL-27 and anti-PD-1 combination therapy induces IL-10 production in T cells. Mice B16 melanoma establishment and treatment protocol were the same as described in FIG. 3 and FIG. 4. Eighteen days after tumor cell injection, mice were sacrificed and their spleens (Panel A) and tumors (Panel B) were isolated and analyzed for the expression of IL-10 in CD8 T cells. Average IL-10-positive T cells from each group of mice (n=5) was shown in the right panel. *p<0.05; **p<0.01 by the student's t test.

FIG. 8. AAV-IL-27 inhibits experimental autoimmune encephalomyelitis. C57BL6/mice (n=5/group) were treated with AAV-IL-27 or AAV-Ctrl viruses (2×10¹¹DVP/mouse, intramuscular injection) (Panel A). Two weeks later, the mice were immunized with MOG peptide to induce experimental autoimmune diseases. The mice were observed every other day and scored on a scale of 0-5 with gradations of 0.5 for intermediate scores: 0, no clinical signs; 1, loss of tail tone; 2, wobbly gait; 3, hind limb paralysis; 4, hind and fore limb paralysis; and 5, death. Panel B depicts a flow cytometric analysis of CD4⁺ cell subsets from the spleens of mice in Panel A at 45 days after AAV infection. The spleen cells were stimulated with PMA for 4 hours in the presence of Golgi blocker and evaluated by intracellular staining of cytokines or Foxp3. Th1, IFNγ-producing cells; TH2, IL4⁺ cells; Th17, IL-17 producing cells; Tr1, IL-10-producing cells. ***P<0.001 by student's t-test.

FIG. 9. AAV-IL-27 inhibits inflammatory bowel diseases (IBD). Colitis was induced by i.p. injection of 0.3×10⁶ CD45RB high CD4⁺ T cells. One week after cell injection, mice were treated with viral vectors 2×10¹¹ DVP/mouse i.m. Panel A depicts body weight changes after T cell transfer. The starting weight is defined as 100%. Panel B depicts an example of gross anatomy of mouse intestine. Panel C depicts clinical scores accumulated based on three parameters: presence or absence of wasting symptoms (0 or 1), extent of colon wall thickness (0-3 for normal, mild, moderate and severe thickening) and feces (0-3 for normal, soft feces, diarrhea and bloody stools, respectively).

FIG. 10. Production of fusion proteins for immunotherapy. FIG. 10 depicts the generation of IL27Fc and CD24IL27Fc. The IL27Fc fusion protein in Panel A is comprised of, from the N to C termini, a signal peptide, EBI3, a GGVPGVGVPGV (SEQ ID NO:7) “PV” linker or a GGGGSGGGGSGGGGS (SEQ ID NO:8) “GS” linker, p24 and Fc. Panel A shows production of two fusion proteins, one with the PV linker (left panel) and the other with the GS linker (right panel). The left panel shows a fusion protein with amino acid sequence in SEQ ID NO:71, while that on the right show the fusion protein with the amino acid sequence in SEQ ID NO:73. The proteins were analyzed by SDS-PAGE under reducing (left lane) and non-reducing (right lane) conditions. The protein yields are provided below the gel photos. Panel B depicts the generation of a CD24IL24Fc fusion protein. The CD24IL24Fc fusion protein comprised of, from the N to C termini, a signal peptide, human CD24, EBI3, a linker, a PV linker peptide, p28 and Fc. Panel B shows the production of a CD24IL24Fc fusion protein with the amino acid sequence in SEQ ID NO:79. The proteins were analyzed by SDS-PAGE under reducing (right lane) and non-reducing (left lane) conditions. The protein yields are provided below the gel photos.

FIG. 11. In vitro activities of the IL27Fc fusion proteins in FIG. 10, panel A. B6 spleen cells (2×10⁵/well) were stimulated with anti-CD3 (1 μg/ml) in the presence of given concentration of IL-27 fusion proteins. The supernatants were harvested on day 3 and analyzed for IL-10 (Panel A) and IFNγ (Panel B) levels. Similar data were obtained from two independent experiments.

FIG. 12. Combination therapy using anti-PD-1 and the IL27Fc fusion protein with amino acid sequence in SEQ ID NO:71 (PV) reduces tumor growth rate and number of CD25⁺Foxp3⁺ Treg. Anti-PD-1 was injected on day 8 (100 μg/injection×5, once every other day), while PV injection was initiated on day 11 after B16.F10 tumor cell challenge (2.5 μg/mouse×5 daily injections). Panel A depicts tumor growth rate reduction. Tumors were measured twice a week by a caliper. Data shown are tumor areas. Panel B depicts Treg reduction. Peripheral blood was harvested on day 7 post-first PV treatment. PBLs were stained with antibodies specific for CD4, CD25 and Foxp3. Data shown are % CD25⁺Toxp3⁺ cells among the CD4 compartment. n=4 for control and 5 for treated groups. *P<0.05, **P<0.01.

FIG. 13 Inhibition of tumor growth and prolongation of survival upon administration of a CD24IL27mFcPV fusion protein (SEQ ID NO:79) alone or in combination with an anti-PD1 mAb. Panel A depicts inhibition of tumor growth. B16 melanoma cells 2×10⁵/mouse) were injected subcutaneously into C57BL/6 mice. Mice were treated with three daily injections with either Fc (200 μg×3), anti-PD-1 (200 μg×3), CD24IL27mFcPV (30 μg×3), or PD-1+ CD24IL27mFcPV, starting at day 8 after tumor cell transplantation. Tumors areas were measured twice a week. P values were determined by two way ANOVA analysis and Bonferroni as a post hoc test. Panel B shows that combination therapy prolongs survival in tumor bearing mice. The data employ a Kaplan Meier Survival analysis, using tumor diameter of 2 cm as the endpoint. P values were determined by a log-rank test.

FIG. 14. Therapeutic effect of IL-27-rAAV in a mouse model of immune dysfunction, polyendocrinopathy, enteropathy, X-linked (IPEX) syndrome. IPEX syndrome is an X-linked recessive disorder with exclusive expression in males. Mutations in the Foxp3 gene cause fatal autoimmune diseases in mice and human. Most affected children die within the first 2 years after birth. Since Scurfy mice with mutations in the Foxp3 gene (Foxp3^{sf}) show a similar pathogenesis as human IPEX, this model was used to determine whether IL-27-rAAV can treat IPEX. As shown in Panel A, a single injection of IL-27-rAAV greatly improved development of mice as demonstrated by increased body weights during perinatal period. Moreover, the treatment nearly doubled survival in the Scurfy mice (Panel B).

### DETAILED DESCRIPTION

Unless defined otherwise, all technical and scientific terms used herein have the same meanings as commonly understood by one of skill in the art to which the disclosed method and compositions belong. It must be noted that as used herein and in the appended claims, the singular forms “a,” “an,” and “the” include plural reference unless the context clearly dictates otherwise. Thus, for example, reference to “a peptide” includes a plurality of such peptides, reference to “the peptide” is a reference to one or more peptides and equivalents thereof known to those skilled in the art, and so forth.

### IL-27 Fusion Protein Compositions

IL-27 is a member of the IL-12 cytokine family that consists of an IL-12 p40-related protein subunit, which is called the EBV-induced 3 (EBI3) protein subunit, and a p35-related subunit, p28. In one aspect, an IL-27 fusion protein for treating proliferative disorders, autoimmune diseases and alloimmune responses includes a CD24 extracellular domain (CD24 ECD), an EBI3 polypeptide subunit and a p28 IL-27 polypeptide subunit, wherein the EBI3 polypeptide subunit and the p28 IL-27 polypeptide subunit are covalently joined by a peptide linker (PL). As used herein, the term “IL-27 fusion protein” broadly refers to any fusion protein described herein, which minimally includes EBI3, p28 and the CD24 extracellular domain. In some embodiments, the fusion protein comprises a modified human p28 peptide (SEQ ID NO:10) or a functional variant thereof. In other embodiments, the fusion protein comprises a wild type human p28 peptide (SEQ ID NO: 142, amino acid residue 28-243 of Accession No. NP_663634) or a functional variant. As used hereinafter, a “functional variant” of a peptide is peptide that differs in amino acid sequence from the original peptide but maintains the biological function of the original peptide. In some embodiments, the function variant maintains at least 70%, 75%, 80%, 85%, 90% or 95% of a biological activity or structure function of the original peptide.

In a particular embodiment, the IL-27 fusion protein comprises, from amino to carboxy terminus, the CD24 extracellular domain, the EBI3 polypeptide subunit, the peptide linker, and the p28 IL-27 polypeptide subunit.

In another embodiment, the IL-27 fusion protein comprises, from amino to carboxy terminus, the CD24 extracellular domain, the p28 IL-27 polypeptide subunit, the peptide linker, and the EBI3 polypeptide subunit.

In certain embodiments, the IL-27 fusion protein may comprise between 2-10 or between 2-5 tandemly arranged copies of the CD24 extracellular domain.

In another embodiment, the IL-27 fusion protein further comprises an immunoglobulin Fc domain. In one embodiment, the immunoglobulin Fc domain comprises an IgG1 heavy chain constant region. In another embodiment, the immunoglobulin Fc domain comprises a mutation abrogating or eliminating binding to an Fcγ receptor.

In other embodiments, the IL-27 fusion protein comprises a GPI-linked glycan or a transmembrane domain for anchoring the fusion protein to cell membranes.

In certain embodiments, the IL-27 fusion protein comprises the CD24 extracellular domain at the amino terminal end and/or a B7-1 transmembrane domain-cytoplasmic tail at the carboxy terminal end.

In some embodiments, the IL-27 fusion protein further comprises an extracellular domain (ECD) from PD-1, PD-L1, PD-L2, B7-1, B7-2, CTLA-4, LAG-3, TIM-3, TIGIT, BTLA, VISTA or a combination thereof. In certain embodiments, the IL-27 fusion protein may comprise between 2-5 tandemly arranged copies of one or more of these ECDs.

The IL-27 fusion proteins of the present disclosure have a modular domain organization with various structural and functional domains from e.g., various nonlimiting sources (e.g., mouse and human) linked together in different configurations. Exemplary domains and their sources for inclusion in the IL-27 fusion proteins are shown in Table 1. The Accession numbers and protein sequence names in Table 1 can be used to find corresponding nucleic acid sequences in nucleic acid databases corresponding to these amino acid sequences. Exemplary nucleic acid sequences encoding the amino acid sequences in SEQ ID NOs:71, 75, 77 and 80 are set forth in SEQ ID NOs:135, 136, 137 and 138, respectively. These nucleic acid sequences in these constructs may be used in the construction of other IL-27 fusion protein encoding constructs described herein.

The domains described in Table 1 may be arranged in various structural configurations, including but not limited to the amino acid sequences in SEQ ID NOs:67-101 (see Table 2). As used herein, the polypeptide or polypeptide domain(s) listed in Table 1 include the corresponding SEQ ID NOs, as well as “functional variants” thereof. As used herein, the term “functional variant” refers to a polypeptide or polypeptide domains maintaining their functional activity and having at least 80%, 85%, 90%, 95%, 98% or 99% sequence identity to the corresponding wild-type polypeptide or polypeptide domains included in the fusion protein, and further includes polypeptide or polypeptide domains truncated at the amino and/or carboxy terminal regions relative to the SEQ ID NO listed, in which the combined truncation(s) account for less than 1%, less than 2%, less than 3%, less than 4%, less than 5%, or less than 10% of the total amino acids in the respective SEQ ID NO listed in Table 1.

Fusion proteins containing amino acid sequences corresponding to SEQ ID NOs:67-101 may be used in the compositions and methods of the present disclosure. In addition, these amino acid sequences may be further rearranged, replaced with functionally substitutable domains, or combined with one or more of the polypeptide domains listed in SEQ ID NOs. 15-66 or otherwise described in the present disclosure. Additional modifications, including deletion of one or more amino acids from the domains described may be employed to increase the biological activity and/or expression of the fusion proteins. In general, it is preferable that at least 90% identity to each domain is preserved, preferably more than 95% identity relative to a particular sequence described herein. In vitro assays, as further described in Example 6 below, provide a useful functional assay that can be used to optimize the fusion protein compositions for clinical use.

TABLE l

| | Mouse polypeptide | | | Human polypeptide | | |
|---|---|---|---|---|---|---|
| | | | SEQ | | | SEQ |
| Polypeptide or | Accession | Amino | ID | Accession | Amino | ID |
| polypeptide domain(s) | No. | Acid Nos. | NO. | No. | Acid Nos. | NO. |
| Ig heavy chain signal | AAA16916 | 1-19 | 1 | AAC98141 | 1-19 | 2 |
| peptide | | | | | | |
| CD24 extracellular | NP_033976 | 27-53 | 3 | NP_037362 | 27-56 | 4 |
| domain (CD24 ECD) | | | | | | |
| IL-27 β subunit (EBI-3) | NP_056581 | 23-228 | 5 | NP_005746 | 27-229 | 6 |
| IL-27 α subunit (p28) | NP_663611 | 28-234 | 9 | | | 10 |
| Ig heavy chain Fc | AAK53870 | 103-324 | 11 | AEV43323 | 3-232 | 12 |
| mutant Ig heavy chain | | | 13 | | | 14 |
| Fc | | | | | | |
| PD-1 ECD | NP_032824 | 21-169 | 15 | NP_005009 | 21-170 | 16 |
| PD-1 ECD/TM | NP_032824 | 21-190 | 17 | NP_005009 | 21-191 | 18 |
| PD-L1 ECD | Q9EP73 | 19-239 | 19 | Q9NZQ7 | 19-238 | 20 |
| PD-L1 ECD/TM | Q9EP73 | 19-260 | 21 | Q9NZQ7 | 19-259 | 22 |
| PD-L2 ECD | Q9WUL5 | 20-221 | 23 | Q9BQ51 | 20-220 | 24 |
| PD-L2 ECD/TM | Q9WUL5 | 20-242 | 25 | Q9BQ51 | 20-241 | 26 |
| B7-1 ECD | Q00609 | 38-246 | 27 | P33681 | 35-242 | 28 |
| B7-1 ECD/TM | Q00609 | 38-268 | 29 | P33681 | 35-263 | 30 |
| B7-2 ECD | P42082 | 24-244 | 31 | P42081 | 24-247 | 32 |
| B7-2 ECD/TM | P42082 | 24-265 | 33 | P42081 | 24-268 | 34 |
| CTLA-4 ECD | P09793 | 36-161 | 35 | P16410 | 36-161 | 36 |
| CTLA-4 ECD/TM | P09793 | 36-182 | 37 | P16410 | 36-182 | 38 |
| mutant CTLA ECD | | | | | | 39 |
| (A295Y/L104E) | | | | | | |
| mutant CTLA ECD/TM | | | | | | 40 |
| (A295Y/L104E) | | | | | | |
| LAG-3 ECD | Q61790 | 23-442 | 41 | P18627 | 29-450 | 42 |
| LAG-3 ECD/TM | Q61790 | 23-463 | 43 | P18627 | 29-471 | 44 |
| TIM-3 ECD | Q8VIM0 | 20-193 | 45 | Q8TDQ0 | 22-202 | 46 |
| TIM-3 ECD/TM | Q8VIM0 | 20-214 | 47 | Q8TDQ0 | 22-223 | 48 |
| TIGIT ECD | P86176 | 29-148 | 49 | Q495A1 | 22-141 | 50 |
| TIGIT ECD/TM | P86176 | 29-169 | 51 | Q495A1 | 22-162 | 52 |
| BTLA ECD | Q7TSA3 | 30-183 | 53 | Q7Z6A9 | 31-157 | 54 |
| BTLA ECD/TM | Q7TSA3 | 30-204 | 55 | Q7Z6A9 | 31-178 | 56 |
| VISTA ECD | AFQ73335 | 32-185 | 57 | AFQ73336 | 32-190 | 58 |
| VISTA ECD/TM | AFQ73335 | 32-213 | 59 | AFQ73336 | 32-216 | 60 |
| CD24 ECD/GPI anchor | NP_033976 | 27-76 | 61 | NP_037362 | 27-80 | 62 |
| signal | | | | | | |
| B7-1 TM | Q00609 | 247-268 | 63 | P33681 | 243-263 | 64 |
| B7-1 TM/Cyto tail | Q00609 | 247-306 | 65 | P33681 | 243-288 | 66 |

TABLE 2
Exemplary IL-27 Fusion Protein Constructs

| SEQ ID | | SEQ ID No. for domains in |
|---|---|---|
| NO | Name | Pro-Protein (NH2—COOH) |
| 67 | mouse IL-27 | 1-5-7-9 |
| 68 | human IL-27 | 1-6-7-10 |
| 69 | mouse IL-27-mouse Fc | 1-5-7-9-111-11 |
| 70 | human IL-27-human Fc | 1-6-7-10-12 |
| 71 | mouse IL-27-mouse mtFc | 1-5-7-9-111-13 |
| 72 | human IL-27-human mtFc | 1-6-7-10-14 |
| 73 | mouse IL-27-mouse Fc | 1-5-8-9-111-11 |
| 74 | human IL-27-human Fc | 1-6-8-10-12 |
| 75 | mouse IL-27-mouse mtFc | 1-5-8-9-111-13 |
| 76 | human IL-27-human mtFc | 1-6-8-10-14 |
| 77 | human CD24-ECD-mouse IL-27-mouse Fc | 1-4-5-7-9-111-11 |
| 78 | human CD24-ECD-human IL-27-human Fc | 1-4-6-7-10-12 |
| 79 | human CD24-ECD-mouse IL-27-mouse mtFc | 1-4-5-7-9-111-13 |
| 80 | human CD24-ECD-human IL-27-human mtFc | 1-4-6-8-10-14 |
| 81 | human CD24-ECD-mouse IL-27-mouse Fc | 1-4-5-8-9-111-11 |
| 82 | human CD24-ECD-human IL-27-human Fc | 1-4-6-8-10-12 |
| 83 | human CD24-ECD-mouse IL-27-mouse mtFc | 1-4-5-8-9-111-13 |
| 84 | human CD24-ECD-human IL-27-human mtFc | 1-4-6-8-10-14 |
| 85 | human IL-27 | 2-6-7-10 |
| 86 | human IL-27-human Fc | 2-6-7-10-12 |
| 87 | human IL-27-human mtFc | 2-6-7-10-14 |
| 88 | mouse CD24-ECD-mouse IL-27-mouse Fc | 1-3-5-7-9-111-11 |
| 89 | human CD24-ECD-human IL-27-human Fc | 2-4-6-7-10-12 |
| 90 | mouse CD24-ECD-mouse IL-27-mouse mtFc | 1-3-5-7-9-111-13 |
| 91 | human CD24-ECD-human IL-27-human mtFc | 2-4-6-7-10-14 |
| 92 | mouse CD24-ECD-mouse IL-27-mouse Fc | 1-3-5-7-9-111-11 |
| 93 | human CD24-ECD-human IL-27-human Fc | 2-4-6-7-10-12 |
| 94 | mouse CD24-ECD-mouse IL-27-mouse mtFc | 1-3-5-7-9-111-13 |
| 95 | human CD24-ECD-human IL-27-human mtFc | 2-4-6-7-10-14 |
| 96 | mouse IL-27-mouse CD24-ECD/gi | 1-5-7-9-111-61 |
| 97 | human IL-27-human CD24-ECD/gpi | 2-6-7-10-111-62 |
| 98 | mouse CD24-ECD-mouse IL-27-mouse TM | 1-3-5-7-9-111-63 |
| 99 | human CD24-ECD-human IL-27-human TM | 2-4-6-7-10-111-64 |
| 100 | mouse CD24-ECD-mouse IL-27-mouse TM/CT | 1-3-5-7-9-111-65 |
| 101 | human CD24-ECD-human IL-27-human TM/CT | 2-4-6-7-10-111-66 |

CD24 is expressed as a glycosyl-phosphatidyl-inositol (GPI)-anchored molecule and has a wide distribution in different lineages. The CD24 extracellular domain (ECD) is heavily glycosylated and is known to interact with members of the family of sialic-acid-binding immunoglobulin-like lectins (Siglecs) to repress inflammatory responses, particularly tissue damage-induced immune responses to danger-associated molecular patterns (DAMPS). For example, CD24 binding to Siglec 10 reduces CD24's association with DAMPS, including high mobility group box 1 (HMGB1), heat shock protein 70 (HSP70) and heat shock protein 90 (HSP90). This serves to negatively regulate their stimulatory activity and inhibit nuclear factor-kappa B (NF-κB) activation. In addition, CD24 binding to Siglec-G is known to attenuate graft-versus-host disease (GVHD).

In addition to its anti-inflammatory activity, the extracellular domain of CD24 (CD24 ECD) provides enhanced expression of the IL-27 fusion proteins described herein. CD24 ECD may be derived from human CD24 or mouse CD24, as they both appear to constitute functionally equivalents in terms of their interactions with DAMPs. The extracellular domain of human CD24 (Accession No. NP_037362) may comprise the amino acid sequence SETTTGTSSNSSQSTSNSGLAPNPTNATTKV (SEQ ID NO:102), SETTTGTSSNSSQSTSNSGLAPNPTNATTK (SEQ ID NO:4), SETTTGTSSNSSQSTSNSGLAPNPTNATT (SEQ ID NO:103), SETTTGTSSNSSQSTSNSGLAPNPTNAT (SEQ ID NO:104), or a functional variant thereof. The extracellular domain of mouse CD24 may comprise the amino acid sequence NQTSVAPFPGNQNISASPNPTNATTRG (SEQ ID NO:105), NQTSVAPFPGNQNISASPNPTNATTR (SEQ ID NO:106), NQTSVAPFPGNQNISASPNPTNATT (SEQ ID NO:107), NQTSVAPFPGNQNISASPNPTNAT (SEQ ID NO:108), or a functional variant thereof.

In some embodiments, the term “CD24 ECD” includes full length extracellular domain of CD24, such as the full length extracellular domain of human CD24 (SEQ ID NO:4) or the full length extracellular domain of mouse CD24 (SEQ ID NO:3), as well as functional variants thereof. A “functional variant” of a CD24 ECD is a polypeptide of 10-60 amino acids that retains at least one or two potential N-linked glycosylation site, and at least 2, 3, 4, 5, 6, 7, 8, 9, 10, 11, 12 or 14 potential 0-linked glycosylation sites of a full length wild-type CD24 ECD.

The CD24 ECD may be fused to the N-terminal side of EBI-PL-p28 (or p28-PL-EBI) or on the C-terminal side of EBI-PL-p28 (or p28-PL-EBI). In some embodiments, CD24 ECD may be fused to both the N- and C-terminal ends of the IL-27 fusion protein. In other embodiments, the IL-27 fusion protein may contain multiple copies of CD24 ECD, tandemly arranged in 2, 3, 4, 5, 6, 7, 8, 9 or 10 copies.

Flexible peptide linkers may be incorporated between any one or more of the polypeptide domains included in the IL-27 fusion protein. The flexible peptide linker includes small amino acids such as glycine, alanine, proline, serine, threonine, and/or methionine residues. Alternatively, the peptide linker may comprise an IgG hinge region. The peptide linker may be between 3 to 40 amino acids, preferably 6 to 15 amino acids in length. Generally, the peptide linker will increase the flexibility of the protein, facilitate adoption of an extended conformation and/or relieve steric hindrance. Modification of linkers, or use of other known linkers may allow for increased stability and/or function of the domains incorporated in the IL-27 fusion proteins.

Preferred peptide linkers are comprised of the amino acids proline, lysine, glycine, alanine, and serine, and combinations thereof. In some embodiments, the peptide linker comprises multiple glycine residues, e.g., from 40% to 70%, or from 70% to 100% of the amino acids in the linker are glycine residues. In other embodiments, the peptide linker comprises multiple serine residues, e.g., from 40% to 70%, or from 70% to 100% of the amino acids in the linker are serine residues (or e.g., alanine and/or proline residues in these amounts). In a particular embodiment, the spacer having the formula [(Gly)n-Ser/Ala]m, where n is from 1 to 4, inclusive, and m is from 1 to 4, inclusive. Specific peptide linkers include, but are not limited to GGVPGVGVPGV (SEQ ID NO:7); GGGGSGGGGSGGGGS (SEQ ID NO:8); GGSSRSSSSGGGGSGGGG (SEQ ID NO:109); GSGRSGGGGSGGGGS (SEQ ID NO:110); GGGGAGGG (SEQ ID NO:111); AAAGGMPPAAAGGM (SEQ ID NO:112); AAAGGM (SEQ ID NO:113); and PPAAAGGMM (SEQ ID NO:114).

In other embodiments, the IL-27 fusion protein further includes an immunoglobulin Fc polypeptide domain. The Fc portion may comprise the hinge region and CH2 and CH3 domains of the human IgG1, IgG2, IgG3, IgG4 or IgA; the hinge region and CH3 and CH4 regions of IgM; or chimera of IgG1, IgG2, IgG3 and IgG4. Preferably the Fc receptor binding domain is of human origin. In other embodiments the Fc domain is from a non-human mammal, including but not limited to, mouse, rat, rabbit, monkey, chimpanzee, hamster etc. In a particular embodiment, the IL-27 fusion protein comprises an immunoglobulin Fc polypeptide domain from the IgG1 heavy chain constant region.

In some embodiments, the Fc region is modified by replacing one or more amino acid residues with different amino acid residue(s) so as to alter the effector functions of the Fc portion. For example, one or more amino acids can be replaced with different amino acid residue(s) such that the Fc portion has an altered affinity for an effector ligand (such as an Fc receptor) and/or the Fc portion is deficient binding an Fcγ receptor (e.g., FcγRI, FcγRII, FcγRIII and FcRn) and consequently unable or significantly hampered in its ability to mediate antibody-dependent cellular cytotoxicity (ADCC). Exemplary Fcγ receptor binding negative IgG1 isotype mutations for inclusion in the Fc region of the IL-27 fusion proteins of the present disclosure include those corresponding to any one of the mutations L17A, L17F, L18A, L18E, D48A, N80A, N80Q, or P114S relative to SEQ ID NO:12, or combinations thereof. In other embodiments, one or more Fc region amino acids may be replaced with one or more residues so that the resulting Fc portion has altered C1q binding and/or reduced or abolished complement dependent cytotoxicity (CDC) or alter the ability of the Fc region to fix complement.

In some embodiments, the IL-27 fusion protein or a polynucleotide encoding the IL-27 fusion protein may further include additional amino acid sequences or polynucleotide sequences encoding for, respectively, a signal peptide sequence, a GPI anchor single sequence, a transmembrane domain, a targeting domain or an affinity tag.

In certain embodiments, the fusion protein may include a GPI anchor signal sequence or a transmembrane domain sequence. Anchoring the IL-27 fusion proteins on the cell plasma membrane by incorporation of a GPI anchor signal or a transmembrane domain sequence can immobilize them in order to increase their in vivo half-life, produce a locally high concentration of the IL-27 fusion proteins and allow for administration of higher doses when, for example, the IL-27 fusion proteins are directly injected into a patient's tumor mass. In addition, purified GPI-anchored proteins possess the ability to integrate spontaneously into cell membranes of virtually any cell.

A wide range of cell-surface proteins, including enzymes, coat proteins, surface antigens, and adhesion molecules, are attached to the plasma membrane via GPI anchors. As used herein, the term “GPI” is used with reference to glycoinositol phospholipids, in particular, glycosylphosphatidlyinositol. These phospholipid-like anchors have a common structure for membrane attachment irrespective of protein function. GPI anchoring units are composed of a linear glycan containing a phosphoethanolamine, three mannose residues, and a nonacetylated glucosamine linked to an inositol phospholipid. GPI is a posttranslationally added lipid anchor; therefore, unlike conventional polypeptide anchors which have different transmembrane domains and connect to specific cytoplasmic extensions, GPI anchors use a common lipid structure to attach to the membrane, which is irrespective of the proteins linked with it.

The residues at the GPI attachment site and immediately following are typically small amino acids such as Ala, Asn, Asp, Gly, Cys or Ser. After the attachment residue, there is a hydrophobic sequence of about 10 to 20 residues starting 7-10 residues after the attachment point. GPI anchor signal sequences have been successfully engineered onto the C-terminus of other un-GPI anchored proteins, and these GPI anchored proteins are coated on the cell surface and are functional.

GPI anchor signal sequences may be obtained from any GPI-anchored protein, including but not limited to enzymes, such as alkaline phosphatase, acetylcholinesterase, 5′ nucleotidase (CO73); adhesion molecules such as lymphocyte function-associated antigen (LFA-3; CD58) and neural cell adhesion molecule (NCAM); complement regulatory proteins such as decay accelerating factor (DAF or CD55), or others such as the Fcγ receptor type III B (Fc-γ-RIII or CD16b), Thy-1 (CD90), Qa-2, Ly-6A and membrane inhibitor of reactive lysis (MIRL or CD59).

The GPI anchor is attached to the protein in the endoplasmic reticulum by transamidation, a reaction in which a C-terminal GPI-attachment signal is cleaved off concomitantly with addition of the GPI moiety. Accordingly, a GPI anchor sequence may be incorporated into the C-terminal end of the IL-27 fusion protein. The native CD24 protein is a GPI-anchored protein. Therefore, in one embodiment, the CD24 GPI anchor signal sequence from the CD24 pro-peptide sequence is incorporated in the nucleic acid coding region of the IL-27 fusion protein at the C-terminal end. In a particular embodiment, the entire portion of the CD24 pro-peptide with the exception of the signal sequence is fused to the C-terminal end of the IL-27 fusion protein (e.g., SEQ ID NOs:60, 62).

In some embodiments, a transmembrane domain may be incorporated in the IL-27 fusion protein for stable attachment to cells. In one embodiment, a heterologous transmembrane domain is incorporated at the C-terminal end of the IL-27 fusion protein. The transmembrane domain may be modeled on other known transmembrane proteins, or may comprise an artificially designed peptide with a high degree of lipophilicity.

Exemplary transmembrane domains include but are not limited to those derived from mouse B7-1 (PPDSKNTLVLFGAGFGAVITVVVIVVI, SEQ ID NO:63), human B7-1 (LLPSWAITLISVNGIFVICCL, SEQ ID NO:64), P-Cadherin (FILPILGAVLALLLLLTLLALLLLV, SEQ ID NO:115), CD2 (IYLIIGICGGGSLLMVEFVALLVFYIT, SEQ ID NO:116), CD40 (ALVVIPIIFGILFAILLVLVFT, SEQ ID NO:117), Contactin (ISGATAGVPTLLLGLVLPAP), SEQ ID NO:118), IL-4 receptor (LLLGVSVSCIVILAVCLLCYVSIT, SEQ ID NO:119), Mannose receptor (VAGVVIIVILLILTGAGLAAYFFY, SEQ ID NO:120), CSF-1 receptor (FLFTPVVVACMSIMALLLLLLLLLL, SEQ ID NO:121), PDGFβ chain receptor (VVVISAILALVVLTIISLIILIMLWQKKPR, SEQ ID NO:122), PDGFα chain receptor (ELTVAAAVLVLLVIVSISLIVLVVTW, SEQ ID NO:123), P-Selectin (LTYFGGAVASTIGLIMGGTLLALL, SEQ ID NO:124), TNFR-1 (TVLLPLVIFFGLCLLSLLFIGLM, SEQ ID NO:125) and VCAM-1 (LLVLYFASSLIIPAIGMIIYFAR, SEQ ID NO:126).

In one embodiment, the IL-27 fusion protein contain the transmembrane domain and cytoplasmic tail (CT) from the co-stimulatory protein, B7-1 at the C-terminal end (Pan et al., Mol. Ther., vol. 20(5):927-937, 2012). Exemplary protein sequences corresponding to the B7-1 transmembrane domain (TM)/cytoplasmic tail (CT) are set forth in SEQ ID NO:65 (mouse) and SEQ ID NO:66 (human).

A cell targeting domain may be incorporated in the IL-27 fusion protein to confer cell-type specific or cell differentiation-specific targeting. The targeting domain may comprise an antibody or antibody derivative, a peptide ligand, a receptor ligand, a receptor fragment, a hormone, etc. In some embodiments, the targeting domain comprises an antibody-derived or peptide-derived targeting domain from a phage display library. Phage display libraries engineered for binding cell surface molecules or receptors are well known to those of skill in the art.

Exemplary antibody or antibody derived targeting domains may include any member of the group consisting of: IgG, antibody variable region; isolated CDR region; single chain Fv molecule (scFv) comprising a VH and VL domain linked by a peptide linker allowing for association between the two domains to form an antigen binding site; bispecific scFv dimer; minibody comprising a scFv joined to a CH3 domain, single chain diabody fragment, dAb fragment, which consists of a VH or a VL domain; Fab fragment consisting of VL, VH, CL and CH1 domains; Fab′ fragment, which differs from a Fab fragment by the addition of a few residues at the carboxyl terminus of the heavy chain CH1 domain, including one or more cysteines from the antibody hinge region; Fab′-SH fragment, which is a Fab′ fragment in which the cysteine residue(s) of the constant domains bear a free thiol group; F(ab′)2, bivalent fragment comprising two linked Fab fragments; Fd fragment consisting of VH and CH1 domains; derivatives thereof, and any other antibody fragment(s) retaining antigen-binding function. Fv, scFv, or diabody molecules may be stabilized by the incorporation of disulphide bridges linking the VH and VL domains. When using antibody-derived targeting agents, any or all of the targeting domains therein and/or Fc regions may be “humanized” using methodologies well known to those of skill in the art.

In other embodiments, an affinity tag may be included to facilitate purification of the IL-27 fusion protein and/or protein of interest by affinity chromatography. The affinity tag may include affinity tag known to those of skill in the art, including, but not limited to, glutathione S-transferase (GST), Histidine tag (e.g., 6×His), maltose binding protein (MBP), Protein A, thioredoxin, ubiquitin, biotin, calmodulin binding peptide (CBP), streptavidin tag, and various immunogenic peptide tags, including FLAG octapeptide tag, hemaglutinin A (HA) tag, myc tag, and the like.

It has been unexpectedly found in the present disclosure that IL-27 fusion proteins in combination with programmed cell death protein-1 (PD-1) blockade can synergistically increase anti-tumor activity and autoimmune or alloimmune responses. Therefore, in one aspect, a pharmaceutical composition for treatment of proliferative diseases, autoimmune diseases and alloimmune responses comprises an IL-27 fusion protein in accordance with the present disclosure in combination with one or more members selected from the group consisting of an anti-PD-1 agent, anti-PD-L1 agent, anti-PD-L2 agent, or combination thereof.

PD-1 and its ligands can negatively regulate immune responses. PD-1 has two ligands, programmed cell death ligand 2 (PD-L1) and programmed cell death ligand 2 (PD-L2), which are members of the B7 family. PD-L1 protein is upregulated on macrophages and dendritic cells (DC) in response to LPS and GM-CSF treatment, and on T cells and B cells upon TCR and B cell receptor signaling, whereas in resting mice, PD-L1 mRNA can be detected in the heart, lung, thymus, spleen, and kidney. PD-L1 is expressed on almost all murine tumor cell lines, including PA1 myeloma, P815 mastocytoma, and B16 melanoma upon treatment with IFN-γ. PD-L1 is also known to bind the co-stimulatory protein, B7-1. This binding interaction can inhibit T cell activation and proliferation, as well as cytokine production. PD-L2 expression is more restricted and is expressed mainly by DCs and a few tumor lines.

PD-1 activity may be interfered with by antibodies that bind selectively to and block the activity of PD-1 or that bind selectively to and prevent binding of PD-L1 or PD-L2 to PD-1. Thus, in certain embodiments, the anti-PD-1 agent, anti-PD-L1 agent or anti-PD-L2 agent is an antibody. Exemplary anti-PD-1 antibodies include, but are not limited to BMS-936558 (under development by Bristol-Meyers Squibb, and also known as MDX-1106 or ONO-4538), CT-011 or pidilizumab (under development by CureTech), MK-3475 (under development by Merck, and also known as SCH 900475). Exemplary anti-PD-L1 antibodies include MPDL3280A or RG7446 (under development by Genentech/Roche) and BMS-936559 (MDX-1105; Bristol-Myers Squibb), a fully human immunoglobulin G4 (IgG4) mAb that binds human PD-L1 with high affinity and blocks PD-L1 binding to both PD-1 and B7-1.

In some embodiments, the anti-PD-1 agent, anti-PD-L1 agent or anti-PD-L2 agent is an siRNA targeting the PD-1 mRNA, the PD-L1 mRNA or the PD-L2 mRNA. An siRNA is a double-stranded RNA that can be engineered to induce sequence-specific post-transcriptional gene silencing of mRNAs. Synthetically produced siRNAs structurally mimic the types of siRNAs normally processed in cells by the enzyme Dicer. Synthetic siRNAs can be designed using well known algorithms and synthesized using a conventional DNA/RNA synthesizer. In addition, siRNAs can be expressed from a suitable expression vector. When expressed from an expression vector, the expression vector is engineered to transcribe a short double-stranded hairpin-like RNA (shRNA) that is processed into a targeted siRNA inside the cell. Synthetic shRNAs may be designed using well known algorithms and synthesized using a conventional DNA/RNA synthesizer.

In other embodiments, the anti-PD-1 agent, anti-PD-L1 agent or anti-PD-L2 agent is a dominant negative mutant form of PD-1, PD-L1 or PD-L2. As used herein, the term “dominant negative mutant” refers to a mutant non-antibody protein that interferes with the binding of the mutant protein to one of its normal binding partners or the mutant binds to its binding partner such that the resulting complex is deficient in its biological activity. For example, a dominant-negative PD-1 may have a mutation such that it is no longer able to bind PD-L1 or PD-L2. Alternatively, a dominant negative protein may bind to its target, but the resulting complex is deficient in signalling. An exemplary dominant negative mutant is AMP-224 (co-developed by Glaxo Smith Kline and Amplimmune). AMP-224 is a recombinant fusion protein comprised of the extracellular domain of PD-L2 and the Fc region of human IgG.

In certain embodiments, the IL-27 fusion protein (or corresponding polynucleotide) of the present disclosure may additionally include or encode the extracellular domain of PD-1, PD-L1 or PD-L2 without the corresponding cytoplasmic tail. Alternatively, a separate dominant negative anti-PD-1 protein, anti-PD-L1 protein or anti-PD-L2 protein (or fusion protein thereof) may be co-administered with the IL-27 fusion protein or a separate polynucleotide expressing such a dominant negative protein may be co-administered with an IL-27 expressing polynucleotide.

In some embodiments, the IL-27 fusion protein comprises a fusion between IL-27 and an anti-PD-1 antibody, anti-PD-L1 antibody, anti-PD-L2 antibody or a fragment thereof. In other embodiments, the IL-27 fusion protein is chemically linked to an anti-PD-1 antibody, anti-PD-L1 antibody, anti-PD-L2 antibody or a fragment thereof.

In some embodiments, the anti-PD-1 agent, anti-PD-L1 agent or anti-PD-L2 agent is a small molecule or peptide mimetic of PD-L1 or PD-L2 that binds PD-1 but does not activate PD-1.

PD-1 antagonists include those described in U.S. Publications 2015/0232555, 2015/0216970, 2015/0210772, 2015/0210769, 2015/0203579, 2013/0280265, 2013/0237580, 2013/0230514, 2013/0109843, 2013/0108651, 2013/0017199, 2012/0251537, 2012/0039906 2011/0271358, US 2011/0171215 and EP 2170959B1.

Any of the above described structural and/or functional domains in the IL-27 fusion proteins of the present disclosure may be separated from one another by one or more of the flexible peptide linkers described herein to facilitate the independent folding of each peptide portion relative to one another and ensure that the individual peptide portions in a fusion protein do not interfere with one another.

### IL-27 Fusion Protein Encoding Polynucleotides

In another aspect, the present disclosure provides polynucleotides encoding an IL-27 fusion protein as described herein. Additional polynucleotides encoding anti-PD-1, anti-PD-L1, and/or anti-PD-L2 agents may be co-administered with the IL-27 fusion protein encoding polynucleotides.

In one embodiment, an expression vector encodes a IL-27 fusion protein (and/or an anti-PD-1/PD-L1/PD-L2 agent) operably linked to one or more regulatory sequences suitable for expressing the fusion protein (and/or anti-PD-1/PD-L1/PD-L2 agent(s)) in a cell.

In certain embodiments, the expression vectors encoding the IL-27 fusion protein alone or in combination with PD-1/PD-L1/PD-L2 agents of the present disclosure are directly administered to a patient to express the IL-27 fusion protein (as well as PD-1/PD-L1/PD-L2 agents) in vivo. The term “in vivo expression vector” refers to a viral or non-viral vector that comprises a polynucleotide encoding the IL-27 fusion protein or the PD-1/PD-L1/PD-L2 agents of the present disclosure in a form suitable for in vivo expression from the polynucleotide(s) in a host cell.

In other embodiments, cells may be transfected or infected with one or more expression vectors encoding the IL-27 fusion protein alone or in combination with one or more expression vectors encoding anti-PD-1, anti-PD-L1 and/or anti-PD-L2 agents of the present disclosure to produce stable or transient cell lines for ex vivo administration into a subject exhibiting a proliferative disorder, autoimmune disease or an undesirable alloimmune response.

A nucleic acid sequence is “operably linked” to another nucleic acid sequence when the former is placed into a functional relationship with the latter. For example, a DNA for a presequence or signal peptide is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous and, in the case of a signal peptide, contiguous and in reading phase. However, enhancers do not have to be contiguous. Linking is accomplished by ligation at convenient restriction sites. If such sites do not exist, synthetic oligonucleotide adaptors or linkers may be used in accordance with conventional practice.

Nucleic acid sequences for expressing the IL-27 fusion protein comprise an amino terminal signal peptide sequence (e.g., SEQ ID NOs:1 and 2), which is removed from the mature protein. Since the signal peptide sequences can affect the levels of expression, the polynucleotides may encode a variety of different N-terminal signal peptide sequences, including but not limited to MELGLSWIFLLAILKGVQC (SEQ ID NO:127); MELGLRWVFLVAILEGVQC (SEQ ID NO:128); MKHLWFFLLLVAAPRWVLS (SEQ ID NO:129); MDWTWRILFLVAAATGAHS (SEQ ID NO:130), MDWTWRFLFVVAAATGVQS (SEQ ID NO:131), MEFGLSWLFLVAILKGVQC (SEQ ID NO:132) and MDLLHKNMKHLWFFLLLVAAPRWVLS (SEQ ID NO:133).

In addition, as noted above, nucleic acid sequences for expressing GPI-linked IL-27 fusion proteins will include a C-terminal GPI anchor signal sequence, which is removed from the mature protein. Exemplary GPI anchor signal sequences may be obtained from protein sequences corresponding to natural GPI-linked proteins, including but not limited to enzymes, such as alkaline phosphatase, acetylcholinesterase, 5′ nucleotidase (CO73); adhesion molecules such as lymphocyte function-associated antigen (LFA-3; CD58) and neural cell adhesion molecule (NCAM); complement regulatory proteins such as decay accelerating factor (DAF or CD55), or others such as the Fcγ receptor type III B (Fc-γ-RIII or CD16b), Thy-1 (CD90), Qa-2, Ly-6A and membrane inhibitor of reactive lysis (MIRL or CD59).

It will be appreciated by those skilled in the art that the design of the expression vector can depend on such factors as the choice of the host cell to be transformed, the level of expression of protein desired, and the like. The expression vectors of the present disclosure can be introduced into host cells to thereby produce proteins or peptides, such as the IL-27 fusion protein and the anti-PD-1, anti-PD-L1 and/or anti-PD-L2 agents of the present disclosure.

The above described “regulatory sequences” refer to DNA sequences necessary for the expression of an operably linked coding sequence in one or more host organisms. The term “regulatory sequences” is intended to include promoters, enhancers and other expression control elements (e.g., polyadenylation signals). Regulatory sequences include those which direct constitutive expression of a nucleotide sequence in many types of host cells or those which direct expression of the nucleotide sequence only in certain host cells (e.g., tissue-specific regulatory sequences). Expression vectors generally contain sequences for transcriptional termination, and may additionally contain one or more elements positively affecting mRNA stability.

The expression vector contains one or more transcriptional regulatory elements, including promoters and/or enhancers, for directing the expression of IL-27 fusion proteins. A promoter comprises a DNA sequence that functions to initiate transcription from a relatively fixed location in regard to the transcription start site. A promoter contains core elements required for basic interaction of RNA polymerase and transcription factors, and may operate in conjunction with other upstream elements and response elements.

As used herein, the term “promoter” is to be taken in its broadest context and includes transcriptional regulatory elements (TREs) from genomic genes or chimeric TREs therefrom, including the TATA box or initiator element for accurate transcription initiation, with or without additional TREs (i.e., upstream activating sequences, transcription factor binding sites, enhancers, and silencers) which regulate activation or repression of genes operably linked thereto in response to developmental and/or external stimuli, and trans-acting regulatory proteins or nucleic acids. A promoter may contain a genomic fragment or it may contain a chimera of one or more TREs combined together.

Preferred promoters are those capable of directing expression in a target cell of interest. The promoters may include constitutive promoters (e.g., HCMV, SV40, elongation factor-1α (EF-1α)) or those exhibiting preferential expression in a particular cell type of interest. Enhancers generally refer to DNA sequences that function away from the transcription start site and can be either 5′ or 3′ to the transcription unit. Furthermore, enhancers can be within an intron as well as within the coding sequence. They are usually between 10 and 300 bp in length, and they function in cis. Enhancers function to increase and/or regulate transcription from nearby promoters. Preferred enhancers are those directing high-level expression in the exosome expressing cell.

The promoter and/or enhancer may be specifically activated either by light or specific chemical inducing agents. In some embodiments, inducible expression systems regulated by administration of tetracycline or dexamethasone, for example, may be used. In other embodiments, gene expression may be enhanced by exposure to radiation, including gamma irradiation and external beam radiotherapy (EBRT), or alkylating chemotherapeutic drugs.

Cell or tissue-specific transcriptional regulatory elements (TREs) can be incorporated into expression vectors to restrict expression to desired cell types. An expression vector may be designed to facilitate expression of the IL-27 fusion protein in one or more cell types. Pol III promoters (H1 or U6) are particularly useful for expressing shRNAs.

In addition, the expression vectors may further include nucleic acid sequence encoding a reporter product or a selectable marker. A reporter product may be used to determine if the gene has been delivered to the cell and is being expressed. Exemplary marker genes include the E. coli lacZ gene, which encodes B-galactosidase, and green fluorescent protein.

In certain embodiments, the expression vector may be engineered to express both the IL-27 fusion protein and an siRNA targeting PD-1, PD-L1 and/or PD-L2. An siRNA is a double-stranded RNA that can be engineered to induce sequence-specific post-transcriptional gene silencing of mRNAs. Synthetically produced siRNAs structurally mimic the types of siRNAs normally processed in cells by the enzyme Dicer. When expressed from an expression vector, the expression vector is engineered to transcribe a short double-stranded hairpin-like RNA (shRNA) that is processed into a targeted siRNA inside the cell. Synthetic siRNAs and shRNAs may be designed using well known algorithms and synthesized using a conventional DNA/RNA synthesizer.

To co-express the IL-27 fusion protein and a suitable shRNA targeting PD-1, PD-L1 and/or PD-L2, a suitable splice donor and splice acceptor sequences may be incorporated for expressing both the IL-27 fusion protein transcript (to produce the IL-27 fusion protein) and the shRNA transcript, whereby the IL-27 fusion protein coding sequence is located upstream (5′ end) of the shRNA coding sequence. Alternatively, when the shRNA is incorporated upstream of the IL-27 coding sequence, an internal ribosome binding sequence (IRES) or a 2A peptide sequence, may be inserted between the shRNA sequence and the downstream coding sequence of the IL-27 fusion protein.

An IRES provides a structure to which the ribosome can bind that does not need to be at the 5′ end of the mRNA. It can therefore direct a ribosome to initiate translation at a second initiation codon within a mRNA, allowing more than one polypeptide to be produced from a single mRNA. A 2A peptide contains short sequences mediating co-translational self-cleavage of the peptides upstream and downstream from the 2A site, allowing production of two different proteins from a single transcript in equimolar amounts. CHYSEL is a non-limiting example of a 2A peptide, which causes a translating eukaryotic ribosome to release the growing polypeptide chain that it is synthesizing without dissociating from the mRNA. The ribosome continues translating, thereby producing a second polypeptide. All that is needed is to clone the coding sequence of 2A, followed by the codon for the first amino acid of the next FMDV protein (e.g., 2B), in frame between the two genes one wishes to co-express. The synthesis of the peptide bond between the last amino acid (Gly) of 2A and the first (Pro) of 2B is skipped, producing an upstream protein with a C-terminal tail of 18aa (2A) and a downstream protein with a Pro at the N-terminus (VKQTLNFDLLKLAGDVESNPGP, SEQ ID NO:134).

In one embodiment, the IL-27 fusion protein is co-expressed with a recombinant antibody against PD-1, PD-L1 and/or PD-L2 by inserting an internal ribosome binding sequence (IRES) or a 2A peptide sequence (such as CHYSEL) between the IL-27 fusion protein coding sequence and the coding sequence for PD-1, PD-L1 or PD-L2. In this embodiment, the IL-27 fusion protein may be incorporated upstream or downstream of the recombinant antibody sequence. In another embodiment, the expression vector comprises two separate expression cassettes, each regulated by its own set of regulatory elements. Alternatively, a plurality of different expression vectors may be constructed for co-administration in which a first expression vector encodes the IL-27 fusion protein and one or more additional expression vectors encode a recombinant antibody against PD-1, PD-L1 and/or PD-L2 (or an shRNA directed against PD-1, PD-L1 and/or PD-L2).

An expression vector may comprise a viral vector or a non-viral vector. A viral vectors may be derived from an adeno-associated virus (AAV), adenovirus, herpesvirus, vaccinia virus, poliovirus, poxvirus, a retrovirus (including a lentivirus, such as HIV-1 and HIV-2), Sindbis and other RNA viruses, alphavirus, astrovirus, coronavirus, orthomyxovirus, papovavirus, paramyxovirus, parvovirus, picornavirus, togaviruses and the like. A non-viral vector is simply a “naked” expression vector that is not packaged with virally derived components (e.g., capsids and/or envelopes).

In certain cases, these vectors may be engineered to target certain diseases and cell populations by using the targeting characteristics inherent to the virus vector or engineered into the virus vector. Specific cells may be “targeted” for delivery of polynucleotides, as well as expression. Thus, the term “targeting”, in this case, may be based on the use of endogenous or heterologous binding agents in the form of capsids, envelope proteins, antibodies for delivery to specific cells, the use of tissue-specific regulatory elements for restricting expression to specific subset(s) of cells, or both.

In some cases, production of recombinant viral particles requires one or more “helper functions”. A “helper function” is a nucleic acid sequence encoding a function required for the replication or packaging of the virus vector. Helper functions may include naturally-occurring nucleic acid sequences, as well as mutated or altered nucleic acid sequences therefrom. Typically, the helper function(s) include nucleotide sequences operably linked to expression control sequences regulating the expression of polypeptides providing the necessary helper functions. The helper functions required for a recombinant virus differ depending upon the type of recombinant virus. The required helper functions for commonly used recombinant viruses are known in the art.

In certain preferred embodiments, the IL-27 fusion proteins are delivered from an adeno-associated virus (AAV) vector. AAVs are small (20-26 nm) replication-defective, nonenveloped viruses, that depend on the presence of a second virus, such as adenovirus or herpes virus or suitable helper functions, for replication in cells. AAV is not known to cause disease and induces a very mild immune response. AAV can infect both dividing and non-dividing cells and may incorporate its genome into that of the host cell. More than 30 naturally occurring serotypes of AAV are available. Many natural variants in the AAV capsid exist, allowing identification and use of AAV vectors with properties specifically suited for the cell targets of delivery. AAV vectors are relatively non-toxic, provide efficient gene transfer, and can be easily optimized for specific purposes. AAV viruses may be engineered using conventional molecular biology techniques to optimize the generation of recombinant AAV particles for cell specific delivery of the IL-27 fusion proteins, for minimizing immunogenicity, enhancing stability, delivery to the nucleus, etc.

Typically, AAV vectors are derived from single-stranded (ss) DNA parvoviruses that are nonpathogenic for mammals. Among the serotypes of AAVs isolated from human or non-human primates, human serotype 2 is the first and best characterized AAV that was developed as a gene transfer vector. Other useful AAV serotypes include AAV1, AAV3, AAV4, AAV5, AAV6, AAV7, AAV8, AAV9, AAV10, AAV11 and AAV12. Generally, recombinant AAV-based vectors have the rep and cap (capsid) viral genes removed (which account for 96% of the viral genome), leaving the two flanking 145-basepair (bp) inverted terminal repeats (ITRs), which are used to initiate viral DNA replication, packaging and integration. Typically, an AAV vector can accommodate a “minigene” of about 4.5 kb in length comprising the transgene (i.e., the fusion protein described herein) operably linked to one or more regulatory elements for expression.

Any suitable AAV serotype may be utilized for the recombinant AAV, including but not limited to AAV1, AAV2, AAV3, AAV4, AAV5, AAV6, AAV7, AAV8, AAV9, AAV10, AAV11, AAV12, and pseudotyped combinations thereof. Pseudotyped (or chimeric) AAV vectors include portions from more than one serotype, for example, a portion of the capsid from one AAV serotype may be fused to a second portion of a different AAV serotype capsid, resulting in a vector encoding a pseudotyped AAV2/AAV5 capsid. Alternatively, the pseudotyped AAV vector may contain a capsid from one AAV serotype in the background structure of another AAV serotype. For example, a pseudotyped AAV vector may include a capsid from one serotype and inverted terminal repeats (ITRs) from another AAV serotype. Exemplary AAV vectors include recombinant pseudotyped AAV2/1, AAV2/2, AAV2/5, AAV2/7, AAV2/8 and AAV2/9 serotype vectors. Unless otherwise specified, the AAV ITRs, and other selected AAV components described herein, may be readily selected from among any AAV serotype, including, without limitation, AAV1, AAV2, AAV3, AAV4, AAV5, AAV6, AAV7, AAV8, AAV9, AAV10, AAV11, AAV12 or other known or as yet unknown AAV serotypes. These ITRs or other AAV components may be readily isolated from an AAV serotype using techniques available to those of skill in the art. In addition, AAV sequences may be isolated or obtained from academic, commercial, or public sources (e.g., the American Type Culture Collection, Manassas, Va.) or may be obtained through synthetic or other suitable means by reference to published sequences such as are available in the literature or in databases such as, e.g., GenBank, PubMed and the like.

Different subtypes of AAV vector or different AAV pseudotypes may be used to express the IL-27 fusion proteins in cells in vitro or in vivo. The types of vectors for in vivo delivery are preferably chosen based on lack of pre-existing immunity in the host to a selected AAV subtypes and stable expression of the fusion protein in vivo.

Exemplary AAV fragments for assembly into vectors and/or helper cells include the capsid subunit proteins, vpl, vp2, vp3, hypervariable regions, and the rep proteins, rep 78, rep 68, rep 52, and rep 40. When providing the AAV rep and cap products, the AAV rep and AAV cap sequences can both be of one serotype origin, e.g., all AAV8 origin or may be derived from multiple AAV serotypes. In one embodiment, the rep and cap sequences are expressed from separate sources (e.g., separate vectors, or a host cell and a vector). In some embodiments, these rep sequences may be fused in frame to cap sequences of a different AAV serotype to form a chimeric AAV vector, such as AAV2/8 described in U.S. Pat. No. 7,282,199.

A recombinant adeno-associated virus (AAV) may be generated by culturing a host cell which contains a nucleic acid sequence encoding an adeno-associated virus (AAV) serotype capsid protein, or fragment thereof, as defined herein; a functional rep gene; a vector composed of, at a minimum, AAV inverted terminal repeats (ITRs) and the fusion protein encoding nucleic acid sequence; and sufficient helper functions to permit packaging of the minigene into the AAV capsid protein. The components required to be cultured in the host cell to package an AAV minigene in an AAV capsid may be provided to the host cell in trans. Alternatively, any one or more of the required components (e.g., minigene, rep sequences, cap sequences, and/or helper functions) may be provided by a stable host cell which has been engineered to contain one or more of the required components using methods known to those of skill in the art. For example, a stable host cell may be generated which is derived from 293 cells (which contain E1 helper functions under the control of a constitutive promoter), but which contains the rep and/or cap proteins under the control of constitutive or inducible promoters.

The minigene, rep sequences, cap sequences, and helper functions required for producing the recombinant AAV (rAAV) of the present disclosure may be delivered or contained in a packaging host cell or helper cell. The selected genetic element may be delivered using any suitable method, including those described herein and any others available in the art. Non-limiting methods of generating rAAV virions are well known in the art.

In some embodiments, the fusion protein transgene is under the control of the regulatory element such as a tissue specific or ubiquitous promoter. In some embodiments, a ubiquitous promoter such as a CMV promoter or a CMV-chicken beta-actin hybrid (CAG) promoter to control the expression of the fusion proteins transgene. In other embodiments, a tissue specific promoter such as a muscle (e.g., liver specific promoter is used to control the expression of the transgene. An exemplary non-limiting examples of a muscle specific promoter is the human muscle creatine kinase (MCK) promoter; a suitable liver specific promoter is phosphoenolpyruvate carboxykinase (PEPCK) promoter.

rAAVs can spread throughout CNS tissue following direct administration into the cerebrospinal fluid (CSF), e.g., via intrathecal and/or intracerebral injection. In some embodiments, rAAVs (such as AAV-9 and AAV-10) cross the blood-brain-barrier and achieve wide-spread distribution throughout CNS tissue of a subject following intravenous administration. In some cases, intravascular (e.g., intravenous) administration facilitates the use of larger volumes than other forms of administration (e.g., intrathecal, intracerebral). Thus, large doses of rAAVs (e.g., up to 1015 rAAV genome copies (GC)/subject) can be delivered at one time by intravascular (e.g., intravenous) administration. Methods for intravascular administration are well known in the art and include, for example, use of a hypodermic needle, peripheral cannula, central venous line, etc.

In certain embodiments, a plurality of different rAAVs may be constructed for co-administration in which a first rAAV encodes the IL-27 fusion protein and a second rAAV encodes a recombinant antibody targeting PD-1, PD-L1 and/or PD-L2.

Non-viral expression vectors can be utilized for non-viral gene transfer, either by direct injection of naked DNA or by encapsulating the IL-27 fusion protein-encoding polynucleotides and/or PD-1/PD-L1/PD-L2 targeting polynucleotides in liposomes, microparticles, microcapsules, virus-like particles, or erythrocyte ghosts. Such compositions can be further linked by chemical conjugation to, for example, microbial translocation domains and/or targeting domains to facilitate targeted delivery and/or entry of nucleic acids into the nucleus of desired cells to promote gene expression. In addition, plasmid vectors may be incubated with synthetic gene transfer molecules such as polymeric DNA-binding cations like polylysine, protamine, and albumin, and linked to cell targeting ligands such as asialoorosomucoid, insulin, galactose, lactose or transferrin.

Alternatively, naked DNA may be employed. Uptake efficiency of naked DNA may be improved by compaction or by using biodegradable latex beads. Such delivery may be improved further by treating the beads to increase hydrophobicity and thereby facilitate disruption of the endosome and release of the DNA into the cytoplasm.

### Methods of Treatment

In one aspect, a method for treating a cell proliferative disorder comprises administering to a subject in need thereof an IL-27 fusion protein or an expression vector expressing an IL-27 fusion protein described herein in an amount effective to treat the proliferative disorder.

In another aspect, a method for treating an autoimmune disease or alloimmune response comprises administering to a subject in need thereof an IL-27 fusion protein or an expression vector expressing an IL-27 fusion protein described herein in an amount effective to treat the autoimmune disease or alloimmune response.

As used herein, the terms “treat” and “treatment” refer to the amelioration of one or more symptoms associated with a cell proliferative disorder, autoimmune disease or alloimmune response; prevention or delay of the onset of one or more symptoms of a cell proliferative disorder, autoimmune disease or alloimmune response; and/or lessening of the severity or frequency of one or more symptoms of cell proliferative disorder, autoimmune disease or alloimmune response.

The terms, “improve”, “increase” or “reduce”, as used in this context, indicate values or parameters relative to a baseline measurement, such as a measurement in the same individual prior to initiation of the treatment described herein, or a measurement in a control individual (or multiple control individuals) in the absence of the treatment described herein.

A “control individual” is an individual afflicted with the same cancer as the individual being treated, who is about the same age as the individual being treated (to ensure that the stages of the disease in the treated individual and the control individual(s) are comparable). The individual (also referred to as “patient” or “subject”) being treated may be a fetus, infant, child, adolescent, or adult human with cancer.

The term “cell proliferative disorder” refers to a disorder characterized by abnormal proliferation of cells. A proliferative disorder does not imply any limitation with respect to the rate of cell growth, but merely indicates loss of normal controls that affect growth and cell division. Thus, in some embodiments, cells of a proliferative disorder can have the same cell division rates as normal cells but do not respond to signals that limit such growth. Within the ambit of “cell proliferative disorder” is a neoplasm or tumor, which is an abnormal growth of tissue. “Cancer” refers to any one of a variety of malignant neoplasms characterized by the proliferation of cells that have the capability to invade surrounding tissue and/or metastasize to new colonization sites, and includes leukemia, lymphoma, carcinoma, melanoma, sarcoma, germ cell tumor and blastoma. Exemplary cancers for treatment with the methods of the instant disclosure include cancer of the brain, bladder, breast, cervix, colon, head and neck, kidney, lung, non-small cell lung, mesothelioma, ovary, prostate, stomach and uterus, leukemia, and medulloblastoma.

The term “leukemia” refers to progressive, malignant diseases of the blood-forming organs and is generally characterized by a distorted proliferation and development of leukocytes and their precursors in the blood and bone marrow. Exemplary leukemias include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, promyelocytic leukemia, Rieder cell leukemia, Schilling's leukemia, stem cell leukemia, subleukemic leukemia, and undifferentiated cell leukemia.

The term “carcinoma” refers to the malignant growth of epithelial cells tending to infiltrate the surrounding tissues and give rise to metastases. Exemplary carcinomas include, for example, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiennoid carcinoma, carcinoma epitheliale adenoides, exophytic carcinoma, carcinoma ex ulcere, carcinoma fibrosum, gelatiniform carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, naspharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, and carcinoma villosum.

The term “sarcoma” refers to a tumor made up of a substance like the embryonic connective tissue and is generally composed of closely packed cells embedded in a fibrillar or homogeneous substance. Exemplary sarcomas include, for example, chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilns' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphomas (e.g., Non-Hodgkin Lymphoma), immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectaltic sarcoma.

The term “melanoma” refers to a tumor arising from the melanocytic system of the skin and other organs. Melanomas include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma subungal melanoma, and superficial spreading melanoma.

Additional cancers include, for example, Hodgkin's Disease, multiple myeloma, neuroblastoma, breast cancer, ovarian cancer, lung cancer, rhabdomyosarcoma, primary thrombocytosis, primary macroglobulinemia, small-cell lung tumors, primary brain tumors, stomach cancer, colon cancer, malignant pancreatic insulanoma, malignant carcinoid, premalignant skin lesions, testicular cancer, thyroid cancer, neuroblastoma, esophageal cancer, genitourinary tract cancer, malignant hypercalcemia, cervical cancer, endometrial cancer, and adrenal cortical cancer.

As used herein, the term “autoimmune disease” refers to a condition in which the immune system of an individual (e.g., activated T cells) attacks the individual's own tissues and cells. The term “alloimmune response” refers to a condition in which the immune system of an individual attack implanted tissue or cells (as in a graft or transplant).

Exemplary autoimmune diseases for treatment with the methods of the instant disclosure include arthritis, alopecia greata, ankylosing spondylitis, autoimmune hemolytic anemia, autoimmune hepatitis, Behcet's disease, Crohn's disease, dermatomyositis, diabetes (Type I), glomerulonephritis, Grave's disease, Guillain-Barre syndrome, inflammatory bowel disorder (IBD), lupus nephritis, multiple sclerosis, myasthenia gravis, myocarditis, pemphigus/pemphigoid, pernicious anemia, polyarteritis nodosa, polymyositis, primary biliary cirrhosis, psoriasis, rheumatoid arthritis, rheumatic fever, sarcoidosis, scleroderma, Sjogren's syndrome, systemic lupus erythematosus (SLE), thyroiditis (such as Hashimoto's thyroiditis and Ord's thyroiditis), ulcerative colitis, uveitis, vitiligo, and Wegener's granulomatosis. Exemplary alloimmune responses for treatment with the methods of the instant disclosure include graft-versus-host disease (GVHD) and transplant rejection.

In one embodiment, the IL-27 fusion protein or IL-27 encoding expression vector is administered alone.

In another embodiment, the IL-27 fusion protein or IL-27 encoding expression vector is administered in combination with one or more anti-PD-1, anti-PD-L1, and/or anti-PD-L2 agents in amount(s) effective to treat the proliferative disorder, autoimmune disease or alloimmune response. The anti-PD-1 agent, anti-PD-L1 agent or anti-PD-L2 agent may be in the form of an antibody, dominant negative protein or siRNA targeting PD-1, PD-L1 or PD-L2.

In certain embodiments, the anti-PD-1 agent, anti-PD-L1 agent or anti-PD-L2 agent comprises a soluble Fc fusion protein comprising the extracellular domain of PD-1, PD-L1 or PD-L2. Alternatively, one or more of the extracellular domains from PD-1, PD-L1 and/or PD-L2 may be incorporated into any of the IL-27 fusion proteins of the present disclosure.

In another embodiment, the IL-27 fusion protein or expression vector is administered in combination with one or more anti-B7-1 agents, anti-B7-2 agents, anti-CTLA-4 agents, anti-LAG-3 agents, anti-TIM-3 agents, anti-TIGIT agents, anti-BTLA agents, anti-VISTA agents in amount(s) effective to treat the proliferative disorder, autoimmune disease or alloimmune response. The anti-B7-1 agent, anti-B7-2 agent, anti-CTLA-4 agent, anti-LAG-3 agent, anti-TIM-3 agent, anti-TIGIT agent, anti-BTLA agent or anti-VISTA agent may be in the form of an antibody, dominant negative protein or siRNA targeting the B7-1, B7-2, CTLA-4, LAG-3, TIM-3, TIGIT, BTLA, and/or VISTA.

In certain embodiments, the anti-B7-1 agent, anti-B7-2 agent, anti-CTLA-4 agent, anti-LAG-3 agent, anti-TIM-3 agent, anti-TIGIT agent, anti-BTLA agent or anti-VISTA agent comprises a soluble Fc fusion protein comprising the extracellular domain of B7-1, B7-2, CTLA-4, LAG-3, TIM-3, TIGIT, BTLA, or VISTA. Alternatively, one or more of the extracellular domains from B7-1, B7-2, CTLA-4, LAG-3, TIM-3, TIGIT, BTLA, and/or VISTA may be incorporated into any of the IL-27 fusion proteins of the present disclosure.

Exemplary anti-PD-1 antibodies include nivolumab (Bristol-Myers Squibb), pembrolizumab (Merck), MK-3475, BMS-936558 (Bristol-Myers Squibb), and pidilizumab/CT-011 (CureTech), MDX-1106 (Medarex).

Exemplary anti-PD-L1 antibodies include BMS-936559 (Bristol-Myers Squibb), MEDI4736 (Medimmune/AstraZeneca), MPDL33280A (Genentech/Roche), MSB0010718C (EMD Serono).

Exemplary anti-CTLA-4 antibodies include ipilimumab and tremelimumab. Exemplary anti-CTLA-4 dominant negative proteins include the humanized fusion protein, Abatacept (Orencia), which comprises the Fc region of IgG1 fused to the CTLA-4 ECD, and Belatacept (Nulojix®), a second generation higher-affinity CTLA-4-Ig variant with two amino acid substitutions in the CTLA-4 ECD relative to Abatacept.

Any suitable route or mode of administration can be employed for providing the patient with a therapeutically or prophylactically effective dose of the fusion protein or expression vector. Exemplary routes or modes of administration include parenteral (e.g., intravenous, intraarterial, intramuscular, subcutaneous, intratumoral), oral, topical (nasal, transdermal, intradermal or intraocular), mucosal (e.g., nasal, sublingual, buccal, rectal, vaginal), inhalation, intralymphatic, intraspinal, intracranial, intraperitoneal, intratracheal, intravesical, intrathecal, enteral, intrapulmonary, intralymphatic, intracavital, intraorbital, intracapsular and transurethral, as well as local delivery by catheter or stent.

A pharmaceutical composition comprising a fusion protein or expression vector in accordance with the present disclosure may be formulated in any pharmaceutically acceptable carrier(s) or excipient(s). As used herein, the term “pharmaceutically acceptable carrier” includes any and all solvents, dispersion media, coatings, antibacterial and antifungal agents, isotonic and absorption delaying agents, and the like that are physiologically compatible. Pharmaceutical compositions may comprise suitable solid or gel phase carriers or excipients. Exemplary carriers or excipients include but are not limited to, calcium carbonate, calcium phosphate, various sugars, starches, cellulose derivatives, gelatin, and polymers such as polyethylene glycols. Exemplary pharmaceutically acceptable carriers include one or more of water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Pharmaceutically acceptable carriers may further comprise minor amounts of auxiliary substances such as wetting or emulsifying agents, preservatives or buffers, which enhance the shelf life or effectiveness of the therapeutic agents.

The fusion proteins or recombinant expression vectors can be incorporated into a pharmaceutical composition suitable for parenteral administration. Suitable buffers include but are not limited to, sodium succinate, sodium citrate, sodium phosphate or potassium phosphate. Sodium chloride can be used to modify the toxicity of the solution at a concentration of 0-300 mM (optimally 150 mM for a liquid dosage form). Cryoprotectants can be included for a lyophilized dosage form, principally 0-10% sucrose (optimally 0.5-1.0%). Other suitable cryoprotectants include trehalose and lactose. Bulking agents can be included for a lyophilized dosage form, principally 1-10% mannitol (optimally 2-4%). Stabilizers can be used in both liquid and lyophilized dosage forms, principally 1-50 mM L-Methionine (optimally 5-10 mM). Other suitable bulking agents include glycine, arginine, can be included as 0-0.05% polysorbate-80 (optimally 0.005-0.01%). Additional surfactants include but are not limited to polysorbate 20 and BRIJ surfactants.

Therapeutic fusion protein preparations can be lyophilized and stored as sterile powders, preferably under vacuum, and then reconstituted in bacteriostatic water (containing, for example, benzyl alcohol preservative) or in sterile water prior to injection. Pharmaceutical composition may be formulated for parenteral administration by injection e.g., by bolus injection or continuous infusion.

The therapeutic agents in the pharmaceutical compositions may be formulated in a “therapeutically effective amount” or a “prophylactically effective amount”. A “therapeutically effective amount” refers to an amount effective, at dosages and for periods of time necessary, to achieve the desired therapeutic result. A therapeutically effective amount of the recombinant vector may vary depending on the condition to be treated, the severity and course of the condition, the mode of administration, whether the antibody or agent is administered for preventive or therapeutic purposes, the bioavailability of the particular agent(s), the ability of the fusion protein or vector to elicit a desired response in the individual, previous therapy, the age, weight and sex of the patient, the patient's clinical history and response to the antibody, the type of the fusion protein or expression vector used, discretion of the attending physician, etc. A therapeutically effective amount is also one in which any toxic or detrimental effects of the recombinant vector is outweighed by the therapeutically beneficial effects. A “prophylactically effective amount” refers to an amount effective, at dosages and for periods of time necessary, to achieve the desired prophylactic result.

Preferably, the polypeptide domains in the fusion protein or polynucleotide are derived from the same host in which they are to be administered in order to reduce inflammatory responses against the administered therapeutic agents.

The fusion protein or expression vector is suitably administered to the patent at one time or over a series of treatments and may be administered to the patient at any time from diagnosis onwards. The fusion protein or expression vector may be administered as the sole treatment or in conjunction with other drugs or therapies useful in treating the condition in question.

As a general proposition, a therapeutically effective amount or prophylactically effective amount of the fusion protein will be administered in a range from about 1 ng/kg body weight/day to about 100 mg/kg body weight/day whether by one or more administrations. In a particular embodiment, each fusion protein or expression vector is administered in the range of from about 1 ng/kg body weight/day to about 10 mg/kg body weight/day, about 1 ng/kg body weight/day to about 1 mg/kg body weight/day, about 1 ng/kg body weight/day to about 100 μg/kg body weight/day, about 1 ng/kg body weight/day to about 10 μg/kg body weight/day, about 1 ng/kg body weight/day to about 1 μg/kg body weight/day, about 1 ng/kg body weight/day to about 100 ng/kg body weight/day, about 1 ng/kg body weight/day to about 10 ng/kg body weight/day, about 10 ng/kg body weight/day to about 100 mg/kg body weight/day, about 10 ng/kg body weight/day to about 10 mg/kg body weight/day, about 10 ng/kg body weight/day to about 1 mg/kg body weight/day, about 10 ng/kg body weight/day to about 100 μg/kg body weight/day, about 10 ng/kg body weight/day to about 10 μg/kg body weight/day, about 10 ng/kg body weight/day to about 1 μg/kg body weight/day, 10 ng/kg body weight/day to about 100 ng/kg body weight/day, about 100 ng/kg body weight/day to about 100 mg/kg body weight/day, about 100 ng/kg body weight/day to about 10 mg/kg body weight/day, about 100 ng/kg body weight/day to about 1 mg/kg body weight/day, about 100 ng/kg body weight/day to about 100 μg/kg body weight/day, about 100 ng/kg body weight/day to about 10 μg/kg body weight/day, about 100 ng/kg body weight/day to about 1 μg/kg body weight/day, about 1 μg/kg body weight/day to about 100 mg/kg body weight/day, about 1 μg /kg body weight/day to about 10 mg/kg body weight/day, about 1 μg /kg body weight/day to about 1 mg/kg body weight/day, about 1 μg /kg body weight/day to about 100 μg/kg body weight/day, about 1 μg /kg body weight/day to about 10 μg/kg body weight/day, about 10 μg/kg body weight/day to about 100 mg/kg body weight/day, about 10 μg/kg body weight/day to about 10 mg/kg body weight/day, about 10 μg/kg body weight/day to about 1 mg/kg body weight/day, about 10 μg/kg body weight/day to about 100 μg/kg body weight/day, about 100 μg/kg body weight/day to about 100 mg/kg body weight/day, about 100 μg/kg body weight/day to about 10 mg/kg body weight/day, about 100 μg/kg body weight/day to about 1 mg/kg body weight/day, about 1 mg/kg body weight/day to about 100 mg/kg body weight/day, about 1 mg/kg body weight/day to about 10 mg/kg body weight/day, about 10 mg/kg body weight/day to about 100 mg/kg body weight/day.

In other embodiments, the fusion protein is administered at a dose of 500 μg to 20 g every three days, or 25 mg/kg body weight every three days.

In other embodiments, each fusion protein is administered in the range of about 10 ng to about 100 ng per individual administration, about 10 ng to about 1 μg per individual administration, about 10 ng to about 10 μg per individual administration, about 10 ng to about 100 μg per individual administration, about 10 ng to about 1 mg per individual administration, about 10 ng to about 10 mg per individual administration, about 10 ng to about 100 mg per individual administration, about 10 ng to about 1000 mg per injection, about 10 ng to about 10,000 mg per individual administration, about 100 ng to about 1 μg per individual administration, about 100 ng to about 10 μg per individual administration, about 100 ng to about 100 μg per individual administration, about 100 ng to about 1 mg per individual administration, about 100 ng to about 10 mg per individual administration, about 100 ng to about 100 mg per individual administration, about 100 ng to about 1000 mg per injection, about 100 ng to about 10,000 mg per individual administration, about 1 μg to about 10 μg per individual administration, about 1 μg to about 100 μg per individual administration, about 1 μg to about 1 mg per individual administration, about 1 μg to about 10 mg per individual administration, about 1 μg to about 100 mg per individual administration, about 1 μg to about 1000 mg per injection, about 1 μg to about 10,000 mg per individual administration, about 10 μg to about 100 μg per individual administration, about 10 μg to about 1 mg per individual administration, about 10 μg to about 10 mg per individual administration, about 10 μg to about 100 mg per individual administration, about 10 μg to about 1000 mg per injection, about 10 μg to about 10,000 mg per individual administration, about 100 μg to about 1 mg per individual administration, about 100 μg to about 10 mg per individual administration, about 100 μg to about 100 mg per individual administration, about 100 μg to about 1000 mg per injection, about 100 μg to about 10,000 mg per individual administration, about 1 mg to about 10 mg per individual administration, about 1 mg to about 100 mg per individual administration, about 1 mg to about 1000 mg per injection, about 1 mg to about 10,000 mg per individual administration, about 10 mg to about 100 mg per individual administration, about 10 mg to about 1000 mg per injection, about 10 mg to about 10,000 mg per individual administration, about 100 mg to about 1000 mg per injection, about 100 mg to about 10,000 mg per individual administration and about 1000 mg to about 10,000 mg per individual administration. The fusion protein or expression vector may be administered daily, every 2, 3, 4, 5, 6 or 7 days, or every 1, 2, 3 or 4 weeks.

In other particular embodiments, the amount of the fusion protein may be administered at a dose of about 0.0006 mg/day, 0.001 mg/day, 0.003 mg/day, 0.006 mg/day, 0.01 mg/day, 0.03 mg/day, 0.06 mg/day, 0.1 mg/day, 0.3 mg/day, 0.6 mg/day, 1 mg/day, 3 mg/day, 6 mg/day, 10 mg/day, 30 mg/day, 60 mg/day, 100 mg/day, 300 mg/day, 600 mg/day, 1000 mg/day, 2000 mg/day, 5000 mg/day or 10,000 mg/day. As expected, the dosage will be dependent on the condition, size, age and condition of the patient.

In certain embodiments, the pharmaceutical composition comprises an effective amount of the recombinant virus, such as rAAV, in an amount comprising at least 10¹⁰, at least 10¹¹, at least 10¹², at least 10¹³, or at least 10¹⁴ genome copies (GC) or recombinant viral particles per kg, or any range thereof. In certain embodiments, the pharmaceutical composition comprises an effective amount of the recombinant virus, such as rAAV, in an amount comprising at least 10¹⁰, at least 10¹¹, at least 10¹², at least 10¹³, at least 10¹⁴, at least 10¹⁵ genome copies or recombinant viral particles genome copies per subject, or any range thereof.

Dosages can be tested in several art-accepted animal models suitable for a particular cancer, automimmune disease or alloimmune response.

Delivery methodologies may also include the use of polycationic condensed DNA linked or unlinked to killed viruses, ligand linked DNA, liposomes, eukaryotic cell delivery vehicles cells, deposition of photopolymerized hydrogel materials, use of a handheld gene transfer particle gun, ionizing radiation, nucleic charge neutralization or fusion with cell membranes, particle mediated gene transfer and the like.

In a further aspect, a method for treating a proliferative disorder, autoimmune disease or an alloimmune response comprises administering to a subject in need thereof a cell expressing an IL-27 fusion protein of the present disclosure alone, or in combination with expression of an anti-PD-1 agent, anti-PD-L1 agent, anti-PD-L2 agent, or combination thereof.

In certain embodiments, the fusion proteins, expression vectors and/or cells may be administered in conjunction with tumor antigens or chimeric antigen-receptor transduced T cells in order to enhance the stimulation of T cells and the resultant therapeutic efficacy.

### Methods for Producing IL-27 Fusion Proteins

Another aspect of the present application relates to a method for producing an IL-27 fusion protein comprising culturing a cell transiently or stably expressing an IL-27 fusion construct; and purifying the IL-27 fusion protein from the cultured cells. Any cell capable of producing a functional IL-27 fusion protein may be used. In preferred embodiments, the IL-27 fusion protein-expressing cell is eukaryotic or mammalian origin. In certain embodiments, the IL-27 fusion protein-producing cell is a human cell. Cells from various tissue cell types may be used to express the IL-27 fusion proteins alone, or in combination with one or more anti-PD-1, anti-PD-L1 and/or anti-PD-L2 agents. In other embodiments, the cell is a yeast cell, an insect cell or a bacterial cell.

In some embodiments, the IL-27 fusion protein-producing cell is stably transformed with a vector expressing the IL-27 fusion protein. In other embodiments, the exosome-producing cell is transiently transfected with a vector expressing the fusion protein.

The expression vector can be introduced into a cell by any conventional method, such as by naked DNA technique, cationic lipid-mediated transfection, polymer-mediated transfection, peptide-mediated transfection, virus-mediated infection, physical or chemical agents or treatments, electroporation, etc. In one embodiment, cells transfected with the vector may be used directly as a source of exosomes (transient transfection). Alternatively, cells may be transfected with a vector expressing a IL-27 fusion protein along with a selectable marker facilitating selection of stably transformed clones expressing the fusion protein and/or. The exosomes produced by such cells may be collected and/or purified according to techniques known in the art, such as by centrifugation, chromatography, etc. as further described in the cited references and Examples herein.

Examples of suitable selectable markers for mammalian cells include dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are CHO DHFR⁻ cells and mouse LTK⁻ cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non-supplemented media.

The second category is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin, mycophenolic acid, or hygromycin. The three examples employ bacterial genes under eukaryotic control to convey resistance to the appropriate drug G418 or neomycin (geneticin), xgpt (mycophenolic acid) or hygromycin, respectively. Others include the neomycin analog G418 and puromycin.

Exemplary IL-27 fusion protein-expressing cells include human Jurkat, human embryonic kidney (HEK) 293, Chinese hamster ovary (CHO) cells, mouse WEHI fibrosarcoma cells, as well as unicellular protozoan species, such as Leishmania tarentolae. In addition, stably transformed, fusion protein producing cell lines may be produced using primary cells immortalized with c-myc or other immortalizing agents.

In one embodiment, the cell line comprises a stably transformed Leishmania cell line, such as Leishmania tarentolae. Leishmania are known to provide a robust, fast-growing unicellular host for high level expression of eukaryotic proteins exhibiting mammalian-type glycosylation patterns. A commercially available Leishmania eukaryotic expression kit is available (Jena Bioscience GmbH, Jena, Germany).

In some embodiments, the cell lines expresses at least 1 mg, at least 2 mg, at least 5 mg, at least 10 mg, at least 20 mg, at least 50 mg, or at least 100 mg of the IL-27 fusion protein/liter of culture.

IL-27 fusion proteins may be isolated from IL-27 fusion protein expressing cells following culture and maintenance in any appropriate culture medium, such as RPMI, DMEM, and AIM V®. The IL-27 fusion proteins can be purified using conventional protein purification methodologies (e.g., affinity purification, chromatography, etc) known to those of skill in the art. In some embodiments, IL-27 fusion proteins are engineered for secretion into culture supernatants for isolation therefrom. The IL-27 fusion proteins can be isolated using conventional protein purification methodologies, including the use of Protein-A or Protein-G immunoaffinity purification of IL-27 fusion proteins comprising Fc binding regions.

The above described methods may be similarly used to express proteins, including antibodies and dominant negative proteins directed against PD-1, PD-L1 and PD-L2.

The present invention is further illustrated by the following examples which should not be construed as limiting. The contents of all references, patents and published patent applications cited throughout this application, as well as the Figures and Tables are incorporated herein by reference.

### EXAMPLES

### Example 1

### Expression of IL-27 Fusion Proteins by Recombinant Adeno-Associated Viral (AAV) Vectors

A cDNA containing EBI3, p28 and a linker therebetween was PCR amplified from pUNO1-mIL27(ebi3p28)(Invivogen) using the primers mIL-27(Xho1): 5′-AAT CTA CTCGAG ATC ACC GGT AGG AGG GCC AA-3′ (SEQ ID NO:139) and mIL-27(EcoRV): 5′-ATG TAT GAT ATC ATG TCG AGC TAG CTT AGG AA-3′ (SEQ ID NO:140). In one embodiment, the PCR amplification product was cut with Xho1/EcoRV and ligated into an AAV-8 expression vector (pAM/CBA-pl-WPRE-bGH, SEQ ID NO:141) under the control of the CMV-chicken beta-actin hybrid (CAG) promoter to form an IL-27 expression vector. The IL-27 expression vector was packaged using an pAAV-RC vector (AAV serotype 8, AAV8) and an adenoviral helper vector in 293K cells as described (Wang Z et al., Nat Biotechnol. 2005; 23(3):321-8; and Qiao C, et al. Hum Gene Ther. 2008; 19(3):241-54) to produce AAV-IL-27, an AAV8-based recombinant AAV vector that expresses the IL-27 fusion protein encoded in the pUNO1-mIL27(ebi3p28) vector.

### Example 2

### Use of AAV-IL-27 as a Therapeutic for Cancer Immunotherapy

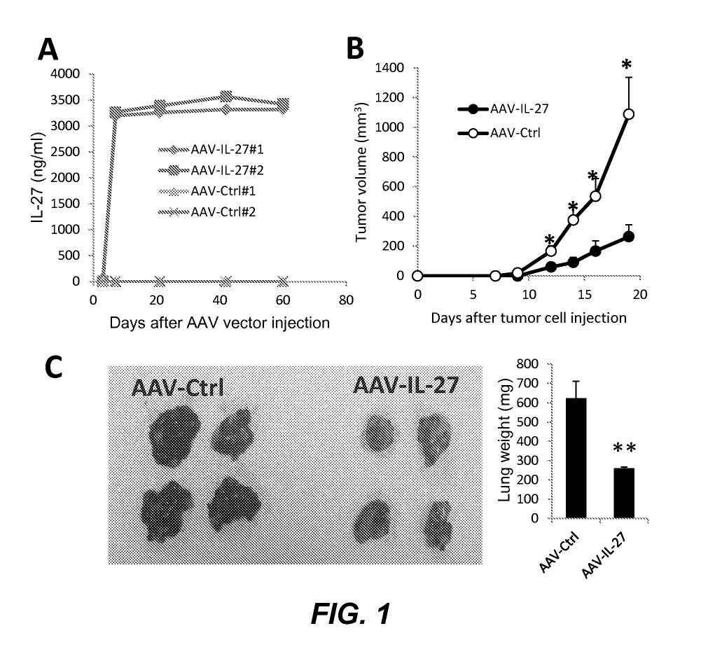

To determine if IL-27 can be used as a potential cancer therapeutic, intramuscular injection (i.m.) of a recombinant adeno-associated viral vector expressing IL-27 (AAV-IL-27) was carried out and the results compared against the control AAV vector (AAV-Ctrl) in Example 1. Intramuscular injection of 2×10¹¹ defective viral particles (DVP)/mouse of AAV-IL-27 achieved high and stable IL-27 production in the peripheral blood of mice (FIG. 1A).

To test whether AAV-IL-27 can be used to treat mice with B16 melanoma, B16.F10 melanoma cells (2×10⁵/mouse) were injected into C57BL/6 mice subcutaneously (s.c.). Four days following the tumor cell injection, mice were injected with AAV-IL-27 or AAV-Ctrl virus i.m. at a dose of 2×10¹¹ DVP/mouse. As shown in FIG. 1B, tumors grew progressively in AAV-Ctrl virus-treated mice, while B16 tumor growth was significantly inhibited in AAV-IL-27-treated mice. In addition, B16.F10 cells were injected into B6 mice intravenously (i.v.), and followed four days later by i.m. injections of a single dose (2×10¹¹ DVP/mouse) of AAV-IL-27 or AAV-Ctrl control virus. As shown in FIG. 1C, mice receiving AAV-IL-27 treatment had significantly reduced tumor foci in their lungs compared to mice treated with AAV-Ctrl virus. As a result, the lung weights of the two groups of mice were significantly different. Thus, AAV-IL-27 treatment significantly inhibits B16 melanoma tumor growth and lung metastasis.

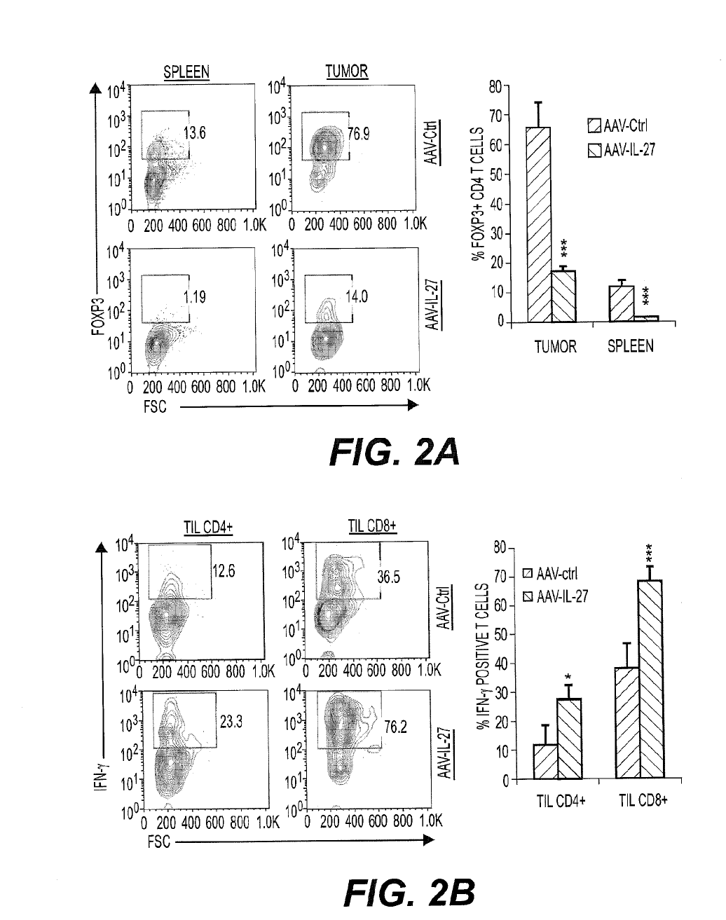

Since IL-27 is known to enhance Th1/Tc1 responses, it was of interest to examine if T cell responses differed between AAV-IL-27 and AAV-Ctrl virus-treated mice. Flow cytometry was used to analyze T cell responses in spleens and tumors from AAV-IL-27-treated mice and controls. Strikingly, FoxP3⁺ Treg cells were found to be depleted in the spleens and were greatly reduced in tumors from AAV-IL-27-treated mice (FIG. 2A). In addition, treatment with AAV-IL-27 was found to significantly increase the percentage of IFN-γ producing CD4⁺ and CD8⁺ T cells in tumors (FIG. 2B). These results show that AAV-IL-27 treatment depletes Treg cells in peripheral lymphoid organs and enhances Th1/Tc1 responses in tumors.

### Example 3

### Combination Therapy Involving AAV-IL-27 and Other Immunotherapeutics

Although AAV-IL-27 treatment reduced Treg cells and enhanced tumor-specific Th1/Tc1 cells, the anti-tumor efficacy of AAV-IL-27 was incomplete, since tumors eventually grew out in all AAV-IL-27-treated mice. Therefore, it was of interest to investigate factors limiting the efficacy of AAV-IL-27 therapy.

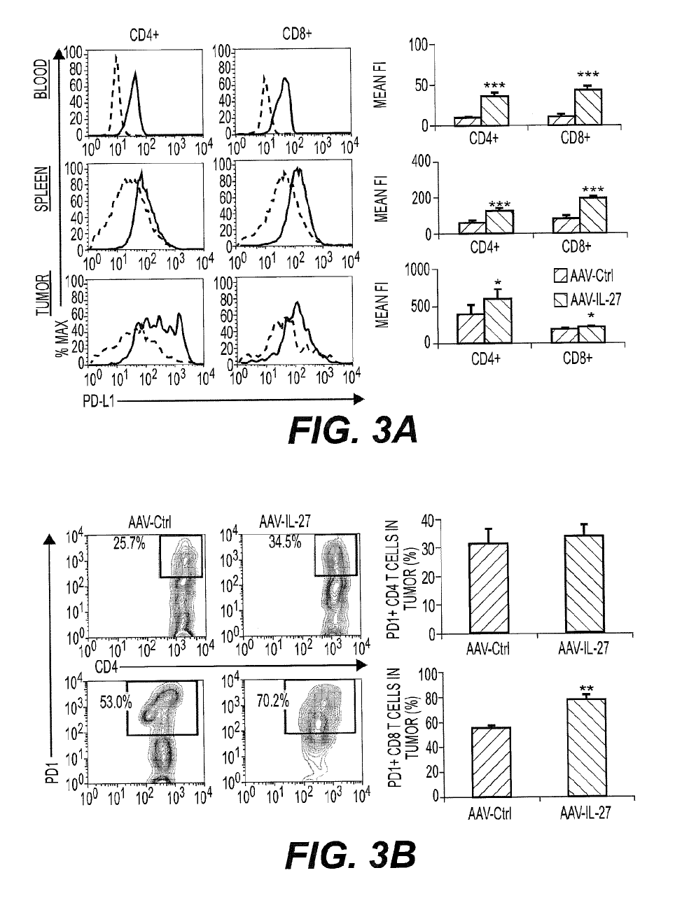

Recently, IL-27 was shown to induce the expression of PD-L1 in T cells which induces T cell tolerance via interaction with PD1 on T cells. This observation was confirmed in CD4⁺ and CD8⁺ T cells from blood, spleen and tumors receiving AAV-IL-27 treatment (FIG. 3A). Additionally, significant proportions of tumor infiltrating CD4⁺ and CD8⁺ T cells were found to express PD1, and AAV-IL-27 treatment was found to up-regulate PD1 expression in T cells (FIG. 3B).

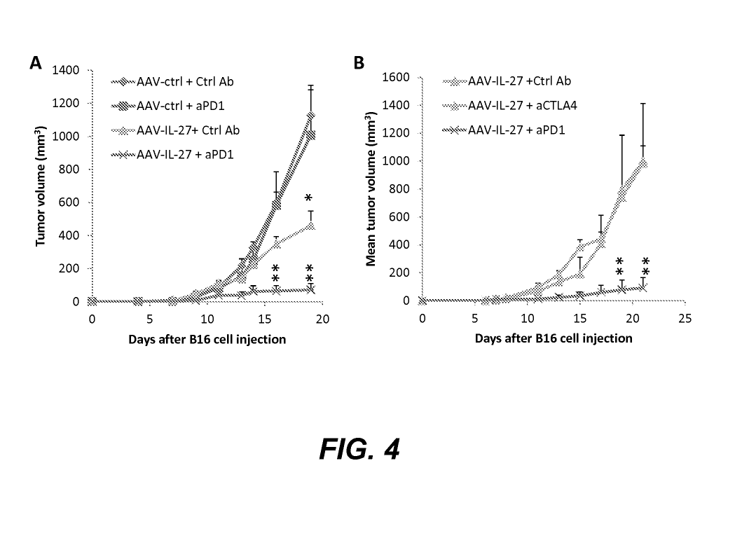

To determine if the IL-27-induced PD-L1 expression in T cells reduced the effectiveness of IL-27 therapy, four groups (n=5 mice/group) of C57BL/6 mice were injected with 2×10⁵ B16.F10 cells s.c. and subsequently received one of the following four different treatments: (1) AAV-Ctrl virus+control mAb; (2) AAV-Ctrl virus+anti-PD1; (3) AAV-IL-27 virus+control mAb; and (4) AAV-IL-27 virus+anti-PD1. AAV viruses were injected into each mouse i.m. at a dose of 2×10¹¹ DVP four days after the tumor cell injection. Antibodies were injected into mice at a dose of 300 μg/mouse i.p. at three day intervals starting on day 4. As shown in FIG. 4A, mice treated with AAV-IL-27 plus control mAb showed significantly reduced tumor growth compared with mice treated with AAV-Ctrl virus+control mAb or AAV-Ctrl virus+anti-PD1. The most significant tumor growth inhibition was observed in mice treated with AAV-IL-27+anti-PD-1 (FIG. 4A). The efficacy of mice treated with AAV-IL-27+anti-PD-1 was compared with mice receiving AAV-IL-27+anti-CTLA-4 treatment. This analysis showed that IL-27 expression in combination with PD1 blockade produced a significant synergistic effect in inhibiting B16 melanoma tumor growth (FIG. 4B). In contrast, AAV-IL-27 administration did not produce any synergistic effect with CTLA4 blockade in inhibiting B16 melanoma tumor growth (FIG. 4B).

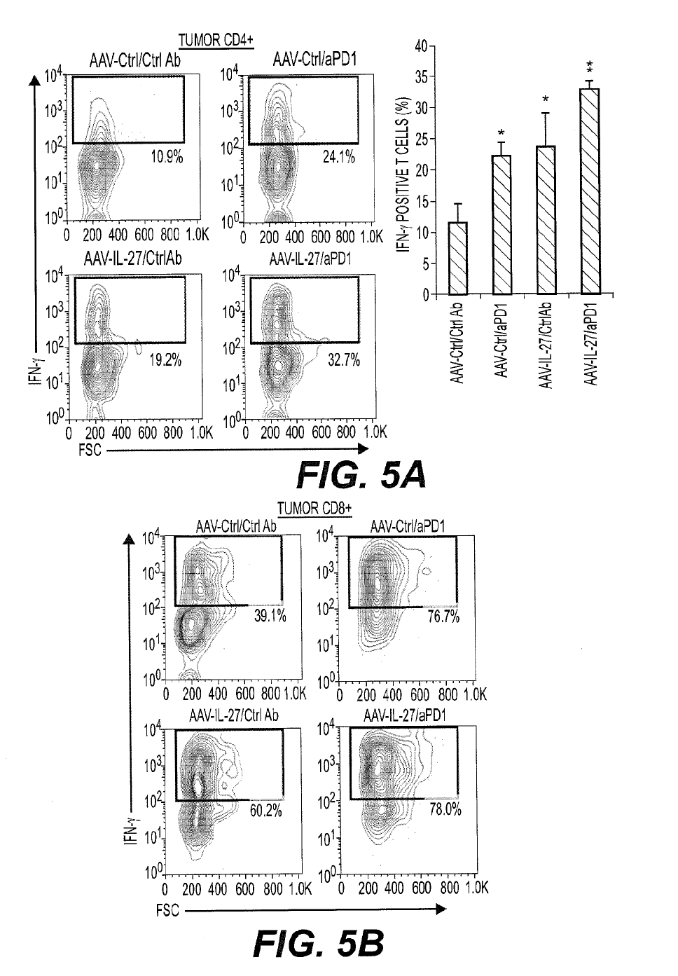

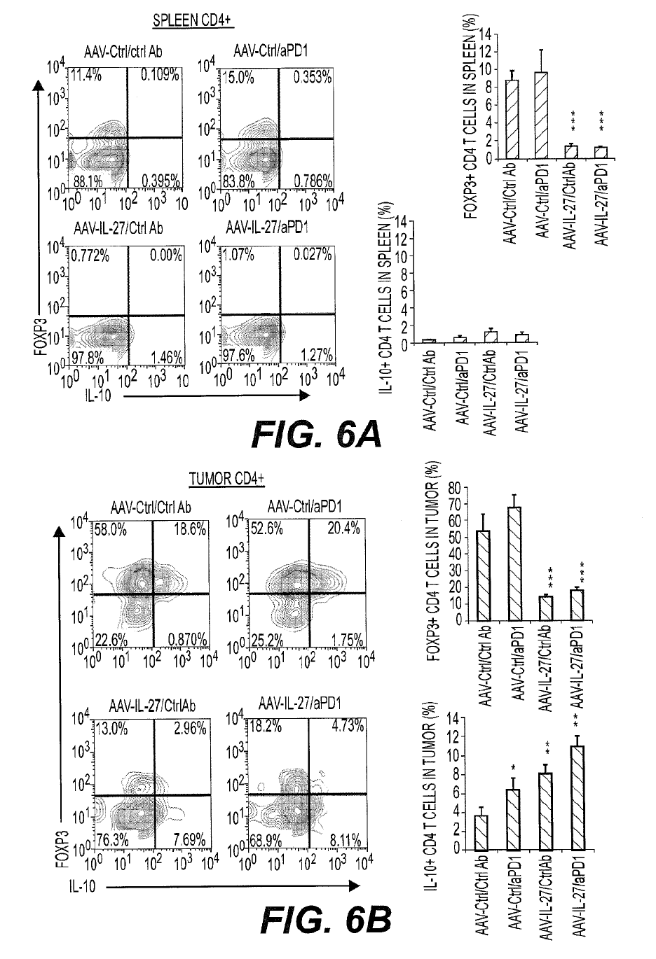

To examine the mechanism by which AAV-mediated IL-27 expression and PD1 blockade combination therapy induced tumor inhibition, tumor infiltrating lymphocytes (TILs) from AAV-IL-27+anti-PD1 treated mice as well as TILs from other control groups of mice were analyzed. AAV-IL-27 or anti-PD1 treatment alone were found to increase the proportions of IFN-γ producing CD4⁺ (FIG. 5A) and CD8⁺ (FIG. 5B) T cells. However, the AAV-IL-27+anti-PD-1 combination therapy greatly increased the proportions of IFN-γ producing CD4⁺ (FIG. 5A) and CD8⁺ (FIG. 5B) T cells. Regardless of anti-PD-1 treatment, however, AAV-IL-27 abrogated FoxP3⁺ Tregs in both spleen (FIG. 6A) and tumor microenvironment (FIG. 6B).

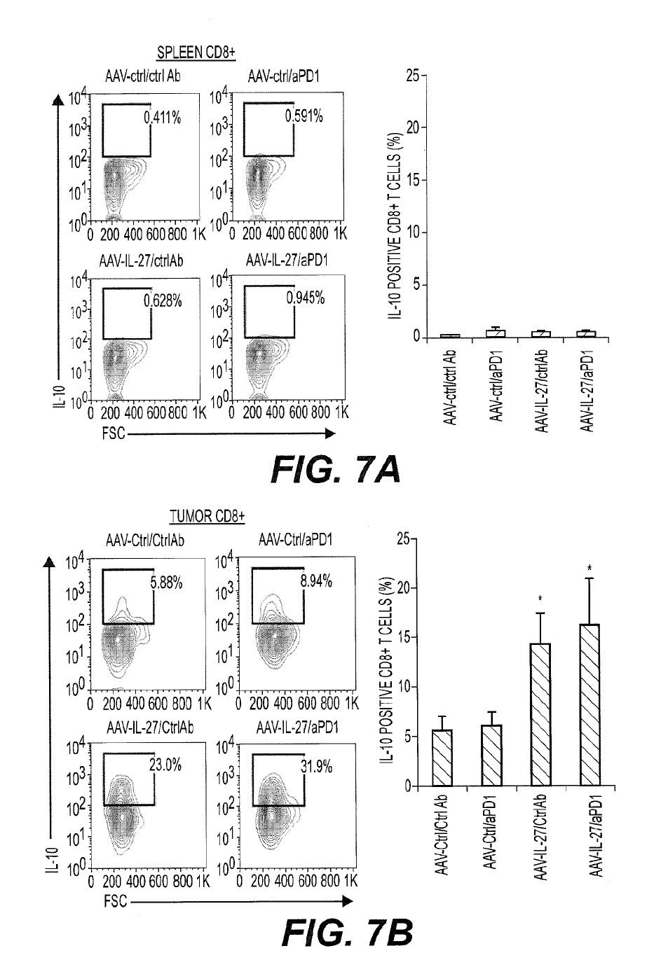

In the tumor microenvironment of B16 tumors, Tregs appeared to be the major source of IL-10 (FIG. 6B). However, in AAV-IL-27 and AAV-IL-27+anti-PD-1-treated mice, the conventional TIL CD4⁺ T cells, but not the conventional CD4⁺ T cells in spleen were induced to produce IL-10, and AAV-IL-27 and anti-PD1 treatment appeared to synergistically induce IL-10 secretion in CD4⁺ T cells (FIG. 6B). While IL-27 failed to induce IL-10 in splenic CD8⁺ T cells (FIG. 7A), robust induction of IL-10 was observed in CD8⁺ T cells in the tumors (FIG. 7B), combination therapy with AAV-IL-27 and anti-PD1 produced a synergistic effect in IL-10 induction in TIL CD8⁺ T cells.

### Example 4

### Use of AAV-IL-27 to Treat Autoimmune Diseases

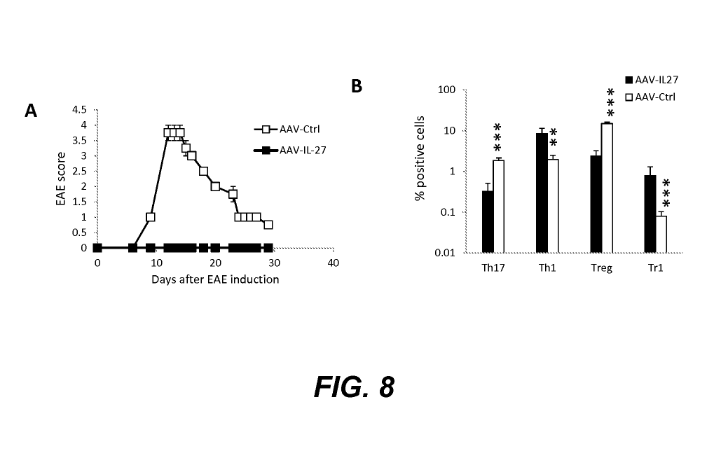

To test whether AAV-IL-27 exacerbates or attenuates autoimmune diseases, experimental autoimmune encephalomyelitis (EAE) was induced in C57BL/6 mice, followed by treatment with control AAV or AAV-IL-27, using the MOG peptide according to conventional procedures. As shown in FIG. 8A, EAE mice treated with control AAV produced robust autoimmune disease. In contrast, EAE mice treated with AAV-IL-27 showed no clinical signs despite increased production of Th1 and reduced production of Treg (FIG. 8B). The reduction of EAE in the AAV-IL-27 treated mice was correlated with a reduction in the percentage of Th17⁺ cells and an increase in the percentage of Tr1⁺ cells (FIG. 8B).

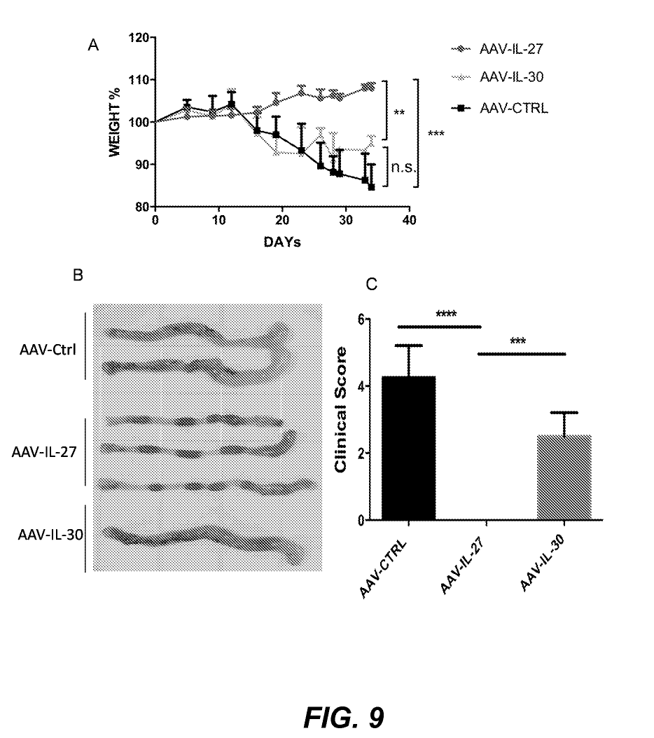

To test the general utility of AAV-IL-27 in treating autoimmune diseases, inflammatory bowel disease was induced in mice by adoptive transfer of CD45RB^{hi} CD4 T cells into immunodeficient Rag1-deficient mice. As shown in FIG. 9, AAV-IL-27 treatment prevented body weight loss (FIG. 9A), reduced colon swelling (FIG. 9B) and ablated all clinical signs of inflammatory bowel disease (FIG. 9C). In contrast, AAV-IL30, which is identical to the p28 subunit of IL-27, had little or no effect.

The results in FIGS. 8 and 9 show that IL-27, and the various forms of fusion proteins comprised of IL-27 disclosed herein, may be used to treat autoimmune diseases. The IL-27 treatments may be used for patients exhibiting active disease episodes or who are under remission for autoimmune disease.

### Example 5

### Compositions and Production of IL-27 Fusion Proteins: GS-IL27Fc, PV-IL27Fc, GS-IL27mFc and PV-IL27mFc

Compared with viral vectors, recombinant proteins are easier to standardize for clinical use and have an easier pathway for regulatory approval. However, several challenges exist in generation of complex proteins. First, IL-27 consists of two chains, and thus may be difficult to correctly fold together if expressed separately. To ensure large scale production and purification in the future, and provide for increased in vivo stability, the IL-27 fusion construct was fused to an immunoglobulin Fc fragment. In certain embodiments, a mutation was introduced to prevent undesirable biological effects associated with Fc binding, including antibody dependent cellular cytotoxicity (ADCC). Accordingly, two IL-27 fusion proteins were produced (SEQ ID NOs:71 and 73).

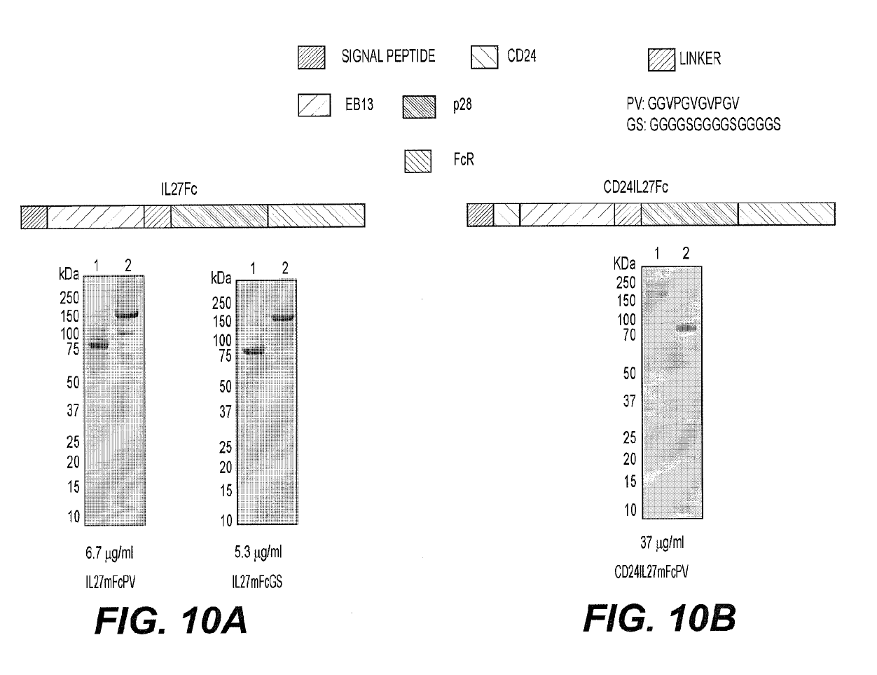

As shown at the top of the figure, the IL27Fc fusion proteins in FIG. 10A are comprised of, from the N to C termini, a signal peptide, EBI3, a PV (SEQ ID NO:7) or GS linker (SEQ ID NO:8), p24 and Fc. FIG. 10A shows production of two fusion proteins, one with the PV linker (left panel) and the other with the GS linker (right panel). The left panel shows a fusion protein with amino acid sequence in SEQ ID NO:71, while that on the right show the fusion protein with the amino acid sequence in SEQ ID NO:73. The proteins were analyzed by SDS-PAGE under reducing (left lane) and non-reducing (right lane) conditions. This analysis confirmed that the fusion proteins form dimers. The protein yields are provided below the gel photos.

Successful purification of these two fusion proteins was achieved by affinity purification; the two fusion proteins (SEQ ID NOs:71, 73) were expressed and purified in similar yields (FIG. 10A). These data showed that a mutant Fc form can be produced without significant adverse effect on purification.

Several additional structural configurations are contemplated, as further described above, including Tables 1 and 2. The use of IgG1 in Example 5 is merely an exemplary IgG isotype source for an IgG Fc (i.e., IgG heavy chain constant region); other Fc subclasses may also be used based on the same design depicted in FIG. 10.

### Example 6

### Functional Assays of IL-27 Fusion Protein Activity

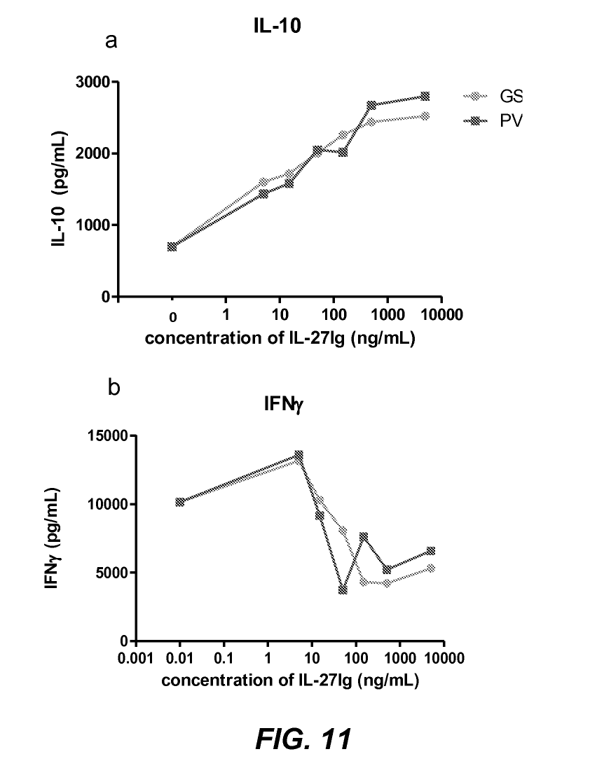

To test the biological activity of the IL-27 fusion proteins, induction of IL10 production was employed for in vitro screening, since this activity is essential for IL-27-induced T cell survival and tumor rejection. As shown in FIG. 11A, both IL-27 fusion proteins described in Example 5 were found to be potent inducers of IL-10 in anti-CD3 stimulated spleen cells. In addition, while low doses of IL-27 fusion protein modestly enhanced IFNγ production, higher doses suppressed IFNγ production, suggesting that at high doses the fusion proteins may be used to suppress autoimmune diseases (FIG. 11B).

These functional assays may be used to monitor the biological activity of the IL-27 fusion protein and may be used to optimize the amino acid composition to ensure high yields and effective biological activities.

### Example 7

### Direct Injection of IL-27 Fusion Proteins In Vivo

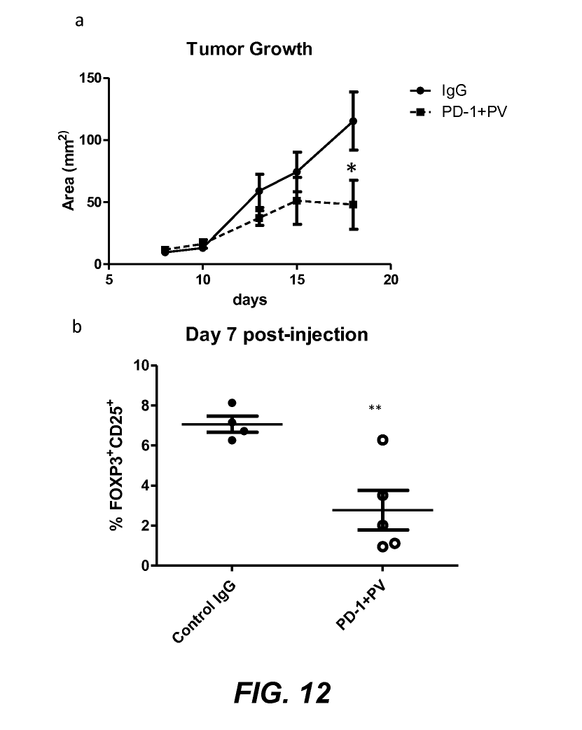

To test whether IL27mFc can promote tumor rejection, C57BL/6 mice were challenged with a B16F10 tumor cell line. Since IL-27 worked synergistically with anti-PD-1 antibodies, and since anti-PD-1 alone does not affect tumor growth in the mice (FIG. 4), combination therapy using direct injection of IL27mFc fusion protein in combination with anti-PD1 mAb was tested. As shown in FIG. 12A, direct injection of IL-27mFc fusion protein in combination with anti-PD1 antibody significantly delayed tumor growth. These data showed that IL-27Fc is a potent inhibitor of tumor growth when used in combination with anti-PD1. Preferably, fusion protein compositions that are most homologous to human components will be used for cancer therapy.

### Example 8

### Production of CD24-IL-27-Fc Fusion Protein

Many cancer immunotherapeutics, such as anti-CTLA-4 mAb cause adverse autoimmune effects. It is therefore preferable to further boost therapeutic effects against autoimmune diseases without reducing cancer therapeutic effects. Therefore, in one embodiment, an IL-27 fusion protein was constructed in which the extracellular domain of CD24 was inserted into IL27Fc (FIG. 10B). As diagrammed in FIG. 10B (top panel), a CD24IL27Fc pro-protein contains a signal peptide, CD24 extracellular domain (CD24 ECD), EBI3 and p28 subunits connected by a linker, and an IgG Fc. One of these proteins containing the amino acid sequence in SEQ ID NO:79 was successfully expressed by transfection into CHO cells. As shown in FIG. 10B (lower panel), this fusion protein was found to be expressed at approximately 6-fold higher levels when compared to IL27Fc (SEQ ID NOs:71, 73). These data demonstrated that inclusion of CD24 ECD can increase the expression and yields, presumably via increased stability and/or solubility.

### Example 9

### CD24IL27Fc as Therapeutic for Cancer

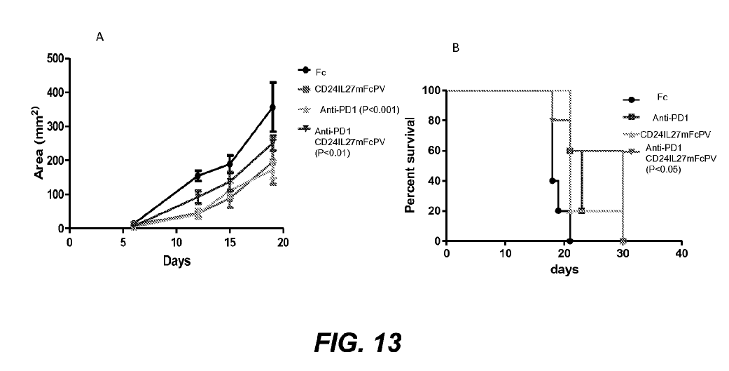

To test the potential of CD24IL27mFc (SEQ ID NO:79) as a therapeutic for cancer therapy, B10F10 tumor cells were transplanted into syngeneic C57BL/6 mice subcutaneously. On day 8, when the tumors were clearly palpable, the mice were treated with either Fc control, a CD24IL27mFc fusion protein (SEQ ID NO:79), anti-PD1, or anti-PD1 in conjunction with CD24IL27mFc. After three daily treatments, the mice were observed for more than 4 weeks. As shown in FIG. 13A, while anti-PD-1 does not reduce tumor growth, CD24IL27mFc, either alone, or in combination with anti-PD1, significantly reduced tumor growth (FIG. 13A). A statistically significant prolongation in survival was observed when both anti-PD-1 and CD24IL27mFc were used (FIG. 13B). Together, these data reduce to practice the concept that CD24IL24Fc can be used either in monotherapy or combination therapy with other immunotherapeutics, such as anti-PD-1.

### Example 10

### Model System for Treatment of IPEX Syndrome

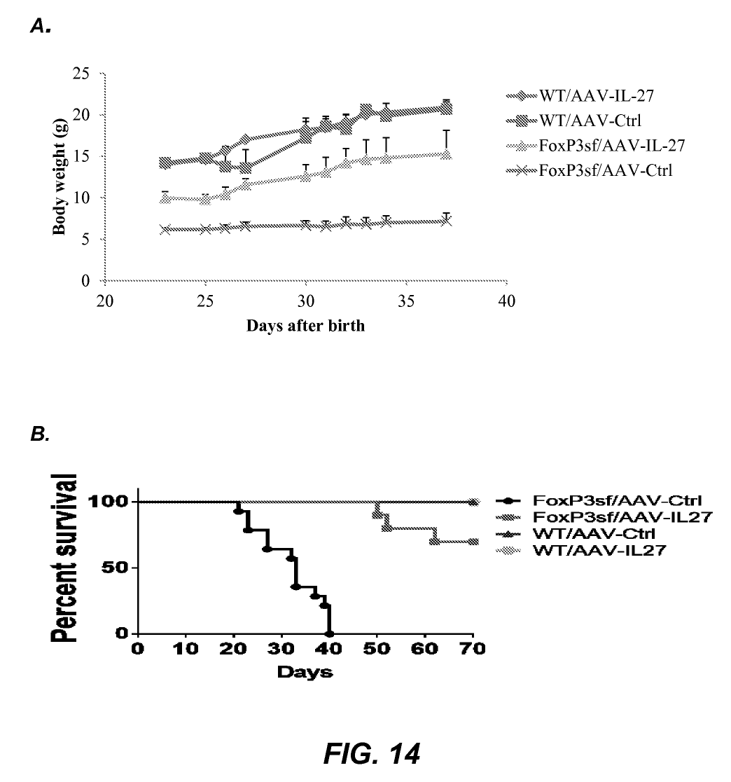

Mutations in the Foxp3 gene cause fatal autoimmune diseases in mice and human. The symptom is called IPEX, for Immune dysfunction, polyendocrinopathy, enteropathy, X-linked (IPEX) syndrome. No cure is available although bone marrow transplantation has emerged as the treatment of choice. Most affected children die within the first 2 years after birth. IPEX syndrome is an X-linked recessive disorder with exclusive expression in males. Since Scurfy mice with mutations in the Foxp3 gene (Foxp3^{sf}) show a similar pathogenesis as human IPEX, this model was used to determine whether IL-27-rAAV can treat IPEX. As shown in FIG. 14, a single injection of IL-27-rAAV greatly improved development of mice as demonstrated by increased body weights during the perinatal period (FIG. 14A). Moreover, the treatment nearly doubled survival in the Scurfy mice (FIG. 14B).

The above description is for the purpose of teaching the person of ordinary skill in the art how to practice the present invention, and it is not intended to detail all those obvious modifications and variations of it which will become apparent to the skilled worker upon reading the description. It is intended, however, that all such obvious modifications and variations be included within the scope of the present invention, which is defined by the following claims. The claims are intended to cover the claimed components and steps in any sequence which is effective to meet the objectives there intended, unless the context specifically indicates the contrary.

1. A fusion protein comprising an CD24 extracellular domain, an EBV-induced 3 (EBI3) polypeptide subunit, and a p28 IL-27 polypeptide subunit, wherein the EBI3 polypeptide subunit and the p28 IL-27 polypeptide subunit are covalently joined by a peptide linker.

2. The fusion protein of claim 1, wherein the protein comprises, from amino to carboxy terminus, the CD24 extracellular domain, the EBI3 polypeptide subunit, the peptide linker, and the p28 IL-27 polypeptide subunit.

3. The fusion protein of claim 1, wherein the protein comprises, from amino to carboxy terminus, the CD24 extracellular domain, the p28 1L-27 polypeptide subunit, the peptide linker, and the EBI3 polypeptide subunit.

4. The fusion protein of claim 1, comprising between 2-5 tandernly arranged copies of the CD24 extracellular domain,

5. The fusion protein of claim 1, further comprising an immunoglobulin Fc domain.

6. The fusion protein of claim 5, wherein the immunoglobulin Fc domain comprises an IgG1 heavy chain constant region.

7. The fusion protein of claim 6, wherein the IgG1 heavy chain constant region comprises a mutation abrogating or eliminating binding to an Fcγ receptor.

8. The fusion protein of claim 1, wherein the protein further comprises a PD-1 extracellular domain, a PD-L1 extracellular domain or a PD-L2 extracellular domain.

9. The fusion protein of claim 1, wherein the fusion protein comprises an amino acid sequence from any one of SEQ ID NOs:77-84 and 88-95.

10. A pharmaceutical composition comprising the fusion protein of claim 1 in combination with one or more members selected from the group consisting of anti-PD-1 agent, anti-PD-L1 agent, anti-PD-L2 agent, or combination thereof.

11-13. (canceled)

14. A polynucleotide comprising a fusion protein sequence encoding the fusion protein of claim 1, wherein the fusion protein sequence comprises an N-terminal signal peptide sequence suitable for secreting the IL-27 fusion protein.

15. The polynucleotide of claim 14, wherein the carboxy-terminal end of the fusion protein sequence comprises a GPI anchor signal sequence suitable for anchoring the IL-27 fusion protein to a cell membrane.

16. An expression vector comprising the polynucleotide of claim 14, wherein the fusion protein sequence is operably linked to one or more regulatory sequences sufficient for expressing the fusion protein in a cell.

17-19. (canceled)

20. A cell comprising the polynucleotide of claim 14, wherein the cell expresses the fusion protein.

21-25. (canceled)

26. A method for treating a proliferative disorder, comprising administering to a subject in need thereof the fusion protein of claim 1 in an amount effective to treat the proliferative disorder in an amount effective to treat the proliferative disorder.

27. A method for treating an autoimmune disease or an alloimmune response, comprising administering to a subject in need thereof the fusion protein of claim 1 in an amount effective to treat the autoimmune disease or the alloimmune response.

28. A method for treating for treating a proliferative disorder, comprising administering to a subject in need thereof the expression vector of claim 16 in an amount effective to treat the proliferative disorder.

29. A method for treating an autoimmune disease or an alloimmune response, comprising administering to a subject in need thereof the expression vector of claim 16 in an amount effective to treat the autoimmune disease or the alloinunune response.

30. (canceled)

31. A method for treating a proliferative disorder, comprising administering to a subject in need thereof the cell of claim 20 in an amount effective to treat the proliferative disorder.

32. A method for treating an autoimmune disease or an alloimmune response, comprising administering to a subject in need thereof the cell of claim 20 in an amount effective to treat the autoimmune disease or the alloimmune response.

33. An AAV vector comprising a coding sequence for a fusion protein comprising an EBV-induced 3 (EBI3) polypeptide subunit and a p28 IL-27 polypeptide subunit covalently joined by a peptide linker.

34. (canceled)
